(19) United States
(12) Patent Application Publication
(10) Pub. No.: US 2023/0355204 A1
(43) Pub. Date: Nov. 9, 2023

(54) WEARABLE ULTRASOUND PATCH FOR MONITORING SUBJECTS IN MOTION USING MACHINE LEARNING AND WIRELESS ELECTRONICS

(71) Applicant: The Regents of the University of California, Oakland, CA (US)

(72) Inventors: Sheng Xu, La Jolla, CA (US); Muyang LIN, La Jolla, CA (US)

(21) Appl. No.: 18/198,982

(22) Filed: May 18, 2023

Related U.S. Application Data
(63) Continuation-in-part of application No. 17/431,572, filed on Aug. 17, 2021.
(60) Provisional application No. 62/811,770, filed on Feb. 28, 2019.

(51) Int. Cl. A61B 8/00

(52) U.S. Cl. CPC A61B 8/4236; A61B 8/565; A61B 8/4427

(57) ABSTRACT

A fully integrated autonomous wearable ultrasonic-system-on-patch (USoP) includes a miniaturized flexible control circuit is designed to interface with an ultrasound transducer array for signal pre-conditioning and wireless data communication. Artificial Intelligence (e.g., machine learning) may be used to track moving tissue targets and assist the data interpretation. In one implementation, the USoP allows continuous tracking of physiological signals from tissues as deep as 164 mm. On mobile subjects, the USoP can continuously monitor physiological signals, including central blood pressure, heart rate, and cardiac output, for as long as e.g., twelve hours. This result enables continuous autonomous surveillance of deep tissue signals.

### CROSS REFERENCE TO RELATED APPLICATION

This application is a continuation-in-part of U.S. application Ser. No. 17/431,572, filed Aug. 17, 2021, which is a 371 National Phase of PCT/US2020/020292, filed Feb. 28, 2020, which claims the benefit of U.S. Provisional Application No. 62/811,770, filed Feb. 28, 2019, the contents of which are incorporated herein by reference in their entireties.

### BACKGROUND

With decades of development in probe fabrication, circuitry design, and algorithm optimization, medical ultrasonography can qualitatively and quantitatively acquire a broad range of physiological information from the human body, including anatomical structures, tissue motion, mechanical properties, and haemodynamics. Compared with other medical imaging methods, such as X-ray computed tomography and magnetic resonance imaging, ultrasonography is safer, less expensive, and more versatile. However, the accessibility and accuracy of ultrasonography face several technical challenges. First, common ultrasound probes are bulky and wired to large control systems, which limits their usage to centralized facilities. Second, those probes need manual placement and maneuvering and require the subjects to remain motionless, introducing operator-dependency. Third, the interpretation of sonographic data requires medical professionals with specialized training and is labor-intensive and error-prone.

Recent advances in point-of-care ultrasound systems have substantially reduced the device size (see FIG. 12). However, they either need manual operations, or require bulky rigid circuits because ultrasound hardware typically requires high power and high bandwidth. The use of bulky rigid probes and circuits create difficulties to cover a large area and conform to highly-curved body surfaces. Emerging wearable ultrasonic probes leveraging soft structural designs can naturally conform to the skin and acquire deep tissue signals in a hands-free manner. Alternatively, integrating rigid ultrasound chips with soft adhesive materials can achieve a reliable interface on the human skin. However, these wearable probes all require cumbersome cables for power and data transmission, which substantially limits the subjects' mobility, making surveillance challenging during dynamic tests or normal daily activities. Developing a fully integrated ultrasonic probe with soft front-end circuits has yet to be demonstrated. Additionally, current wearable ultrasound technologies can lose track of a target tissue during subject motion, because the device on the skin surface shifts its position relative to deep tissues. Thus, they require frequent manual repositioning and only allow point-in-time examinations. Moreover, with the large amount of data generated from continuous surveillance, the front-end circuits and back-end processing units would be overwhelmed. Therefore, an important milestone in the development of wearable ultrasound technology is to realize a fully integrated wireless system that can track a moving target and automate data acquisition and processing.

### SUMMARY

In one aspect of the subject matter disclosed herein, a fully integrated autonomous wearable ultrasonic-system-on-patch (USoP) is presented herein. A miniaturized flexible control circuit is designed to interface with an ultrasound transducer array for signal pre-conditioning and wireless data communication. Artificial Intelligence (e.g., machine learning) may be used to track moving tissue targets and assist the data interpretation. In one implementation, we demonstrate that the USoP allows continuous tracking of physiological signals from tissues as deep as 164 mm. On mobile subjects, the USoP can continuously monitor physiological signals, including central blood pressure, heart rate, and cardiac output, for as long as twelve hours. This result enables continuous autonomous surveillance of deep tissue signals.

In one particular aspect of the subject matter disclosed herein, a system is presented for monitoring a physiologic parameter. The system includes a conformal ultrasonic transducer array, an analog front end circuit and a digital circuit. The conformal ultrasonic transducer array is located on a flexible substrate. The analog front end circuit is located on the flexible substrate and is further coupled to the conformal ultrasonic transducer array. The analog front end circuit is configured to at least cause the conformal ultrasonic transducer array to generate ultrasonic acoustic waves and receive reflected ultrasonic acoustic waves. The digital circuit is located on the flexible substrate and is further coupled to the analog front end circuit. The digital circuit is configured to at least: (i) control the analog front end circuit at least in its generation of ultrasonic acoustic waves using a plurality of sensing channels; (ii) transmit data concerning the received reflected ultrasonic acoustic waves to a back-end computing environment that dynamically selects a monitoring channel in real-time from among the plurality of sensing channels; and (iii) receive in real-time at least an identifier of the selected monitoring channel from the back-end computing environment and cause the analog front-end circuit to generate ultrasonic acoustic waves using the selected monitoring channel to perform the monitoring of the physiological parameter.

In accordance with another aspect of the subject matter disclosed herein, the selected monitoring channel received by the digital circuit is dynamically selected in real-time by the back-end computing environment at least in part using artificial intelligence techniques.

In accordance with another aspect of the subject matter disclosed herein, the artificial intelligence techniques identify sensing channels that cause reflected ultrasonic acoustic waves to be reflected from specified tissue that is to be monitored.

In accordance with another aspect of the subject matter disclosed herein, the artificial intelligence techniques identify sensing channels that cause ultrasonic acoustic waves to be transmitted through specified tissue that is to be monitored.

In accordance with another aspect of the subject matter disclosed herein, the artificial intelligence techniques employ models that are generalizable to allow physiological monitoring to be performed on different subjects.

In accordance with another aspect of the subject matter disclosed herein, the selected monitoring channel received by the digital circuit is dynamically selected in real-time by the back-end computing environment to accommodate motion of tissue relative to the conformal ultrasonic transducer array.

In accordance with another aspect of the subject matter disclosed herein, the physiological parameter being monitored is selected from the group including blood pressure, heart rate, pulse wave velocity, stroke volume, cardiac output, augmentation index, and expiratory volume.

In accordance with another aspect of the subject matter disclosed herein, the digital circuit includes a wireless communication circuit for communicating with the back-end computing environment.

In accordance with another aspect of the subject matter disclosed herein, the wireless communication circuit is a Wi-Fi communication circuit.

In accordance with another aspect of the subject matter disclosed herein, the digital circuit is further configured to transmit an indication of the reflected ultrasonic acoustic waves arising from use of the selected monitoring channel to an external computing environment for display thereon.

In accordance with another aspect of the subject matter disclosed herein, the digital circuit is further configured to present an indication of the reflected ultrasonic acoustic waves arising from use of the selected monitoring channel.

In accordance with another aspect of the subject matter disclosed herein, the back-end computing environment is the same or different from the external computing environment.

In accordance with another aspect of the subject matter disclosed herein, the backend computing environment is configured to measure a shift, the shift in the time domain, in a detected peak of the received reflected acoustic wave, the shift due to movement of an organ or tissue, and wherein the displayed indication of the monitored physiologic parameter is based on the measured shift.

In accordance with another aspect of the subject matter disclosed herein, the analog front end is further configured to steer or direct the generated ultrasonic acoustic waves toward an organ, tissue, or location of interest, the steering or directing by beamforming.

In accordance with another aspect of the subject matter disclosed herein, the steering includes dynamically adjusting a time-delay profile of individual transducer activation in the transducer array.

In accordance with another aspect of the subject matter disclosed herein, the transducer array is selected from the group including a piezoelectric array, a piezoelectric micromachined ultrasonic transducer (PMUT) array or a capacitive micromachined ultrasonic transducer (CMUT) array.

In accordance with another aspect of the subject matter disclosed herein, the analog front end circuit includes a multiplexer for selecting from among all sensing channels that are used to generate the ultrasonic acoustic wave and perform monitoring.

In accordance with another aspect of the subject matter disclosed herein, the artificial intelligence techniques are machine learning techniques.

In accordance with another aspect of the subject matter disclosed herein, the system further includes comprising a battery (e.g., a lithium-polymer battery) located on the flexible substrate for powering the analog front end circuit and the digital circuit up to e.g., 12 hours.

In accordance with another aspect of the subject matter disclosed herein, a system for monitoring a physiologic parameter is presented. The system includes a conformal ultrasonic transducer array, an analog front end circuit and a digital circuit. The conformal ultrasonic transducer array is located on a flexible substrate. The analog front end circuit is located on the flexible substrate and is further coupled to the conformal ultrasonic transducer array. The analog front end circuit is configured to at least cause the conformal ultrasonic transducer array to generate ultrasonic acoustic waves and receive reflected and/or transmitted ultrasonic acoustic waves. The digital circuit is located on the flexible substrate and is further coupled to the analog front end circuit. The digital circuit is configured to at least: (i) control the analog front end circuit at least in its generation of ultrasonic acoustic waves using a plurality of sensing channels; (ii) dynamically select a monitoring channel in real-time from among the plurality of sensing channels; and (iii) cause the analog front-end circuit to use the selected monitoring channel to perform the monitoring of the physiological parameter.

In accordance with another aspect of the subject matter disclosed herein, a method is presented for monitoring a physiologic parameter. The method includes: (a) determining a location of interest, the location associated with the physiologic parameter to be monitored; (b) transmitting ultrasonic acoustic waves toward the location of interest and receiving reflected ultrasonic acoustic waves from the location of interest using a plurality of sensing channels; (c) dynamically selecting a monitoring channel in real-time from among the plurality of sensing channels; (d) monitoring the physiological parameter in real-time by transmitting ultrasound acoustic waves toward the location of interest and receiving reflected ultrasonic acoustic waves using the selected monitoring channel; (e) outputting data reflective of the monitored physiological parameter; and f. wherein at least steps (b) and (d) are performed by components within the conformable integrated wearable device.

In accordance with another aspect of the subject matter disclosed herein, step (c) is also performed by components within the integrated conformable wearable device.

In accordance with another aspect of the subject matter disclosed herein, step (c) is performed by a back-end computing environment located external to the integrated conformable wearable device and the method further comprises: transmitting data concerning the received reflected/transmitted ultrasonic waves from the conformable integrated wearable device to the back-end computing device; and receiving from the back-end computing device at least an identifier of the selected monitoring channel.

In accordance with another aspect of the subject matter disclosed herein, the selected monitoring channel is dynamically selected in real-time at least in part using artificial intelligence techniques.

In accordance with another aspect of the subject matter disclosed herein, the artificial intelligence techniques identify sensing channels that cause reflected ultrasonic acoustic waves to be reflected from specified tissue that is to be monitored.

In accordance with another aspect of the subject matter disclosed herein, the artificial intelligence techniques identify sensing channels that cause ultrasonic acoustic waves to be transmitted to specified tissue that is to be monitored.

In accordance with another aspect of the subject matter disclosed herein, the artificial intelligence techniques employ models that are generalizable to allow physiological monitoring to be performed on different subjects.

In accordance with another aspect of the subject matter disclosed herein, the selected monitoring channel is dynamically selected in real-time to accommodate motion of tissue relative to the conformal ultrasonic transducer array.

In accordance with another aspect of the subject matter disclosed herein, the physiological parameter being monitored is selected from the group including blood pressure, heart rate, stroke volume, cardiac output, augmentation index, and expiratory volume.

In accordance with another aspect of the subject matter disclosed herein, the integrated conformable wearable device includes a wireless communication circuit for communicating with the back-end computing environment.

In accordance with another aspect of the subject matter disclosed herein, the wireless communication circuit is a Wi-Fi communication circuit.

In accordance with another aspect of the subject matter disclosed herein, the displaying includes transmitting an indication of the reflected ultrasonic acoustic waves arising from use of the selected monitoring channel to an external computing environment for display thereon.

In accordance with another aspect of the subject matter disclosed herein, the method further comprises measuring a shift, the shift in the time domain, in a detected peak of the received reflected acoustic wave, the shift due to movement of an organ or tissue, and wherein the displaying of data reflective of the monitored physiologic parameter is based on the measured shift.

In accordance with another aspect of the subject matter disclosed herein, the method further comprises steering or directing the transmitted ultrasonic acoustic waves toward an organ, tissue, or location of interest, the steering or directing by beamforming.

In accordance with another aspect of the subject matter disclosed herein, the artificial intelligence techniques are machine learning techniques.

In accordance with another aspect of the subject matter disclosed herein, the outputting of data reflective of the monitored physiological parameter includes displaying data reflective of the monitored physiological parameter.

In accordance with another aspect of the subject matter disclosed herein, a method for monitoring a physiologic parameter includes: (a) determining a location of interest, the location associated with the physiologic parameter to be monitored; (b) transmitting ultrasonic acoustic waves toward the location of interest and receiving resulting ultrasonic acoustic waves transmitted through the location a interest using a plurality of sensing channels; (c) dynamically selecting a monitoring channel in real-time from among the plurality of sensing channels; (d) monitoring the physiological parameter in real-time by transmitting ultrasound acoustic waves toward the location of interest and receiving resulting ultrasonic acoustic waves transmitted through the location of interest using the selected monitoring channel; (e) outputting data reflective of the monitored physiological parameter; and (f) wherein at least steps (h) and (d) are performed by components within the conformable integrated wearable device.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure.

### BRIEF DESCRIPTION OF THE DRAWINGS

FIGS. 1a-1d provide an overview of the fully integrated ultrasonic-system-on-patch (USoP).

FIGS. 2a-2e illustrate the monitoring and analysis of tissue interface motions using the USoP.

FIGS. 3a-3f illustrate the autonomous and continuous blood pressure recording in a moving subject.

FIGS. 4a-4f illustrate the continuous monitoring process during exercise.

FIGS. 5a-5d present data characterizing bandwidth, axial resolution, and penetration of the stretchable ultrasonic probes.

FIGS. 6a-6d shows schematics and the control sequence for ultrasonic sensing.

FIGS. 7a-7d illustrate deformation of the packaged USoP.

FIGS. 8a-8g illustrate the skin integration of the conformal USoP device.

FIGS. 9a-9d show pulse wave propagation paths and pulse wave velocity (PWV) measurements.

FIGS. 10a-10e show the validation metrics of four models on ideal and compromised image datasets.

FIGS. 11a-11c illustrate the continuous monitoring process during high-intensity interval training (HIIT).

FIGS. 12a-12d show ultrasonic devices for wearable or point-of-care applications.

FIGS. 13a-13b show probe layout designs for reducing noise coupling.

FIG. 14 demonstrates the improved axial resolution that arises with a backing layer.

FIGS. 15a-15b show radiofrequency signals collected from the carotid artery with and without gel.

FIGS. 16a-16b show results of a durability test of the soft probe.

FIGS. 17a-17e show layout and beam profile designs of three soft probes.

FIGS. 18a-18d show characterization data for the detachable ACF connection.

FIGS. 19a-19c show layout designs of the fPCB circuit.

FIG. 20 shows schematic connections of the analog front-end and wireless data acquisition module.

FIGS. 21a-21d illustrate the foldability of the fPCB.

FIGS. 22a-22c show designs of the mold for the elastomeric package.

FIGS. 23a-23e show mechanical simulations of the fPCB and the elastomeric package.

FIG. 24 compares the raw signal frequency and circuit sampling rate of representative wearable physiological monitors.

FIGS. 25a-25c illustrate the wireless transmission of the ultrasonic signals via Wi-Fi.

FIGS. 26a-26b illustrate the power consumption and battery life of the USoP.

FIGS. 27a-27d illustrate multi-mode sensing with wearable ultrasonic probes.

FIGS. 28a-28g illustrate the lateral and elevational resolution of the soft probes. a

FIGS. 29a-29e illustrate the transmission beam patterns with elevational deformation.

FIGS. 30a-30b show simulated B-mode images of point sources with azimuthal bending.

FIGS. 31a-31c illustrate tissue interfacial motion detection using the auto-correlation method.

FIGS. 32a-32g show probe positions and acoustic views of different bio-interface measurements.

FIGS. 33a-33b show fractional shortening measurements using a commercial ultrasonic system.

FIGS. 34a-34b show calculations of expiratory volumes.

FIGS. 35a-35b illustrate model training and validation with modified datasets

FIGS. 36a-36e illustrate the process of classifying carotid artery images by the image processing and logistic model. a

FIG. 37 illustrates the statistical validation of the prediction of the best channel for carotid artery sensing against the ground truth.

FIGS. 38a-38c illustrate the process of recording head rotation.

FIGS. 39a-39b illustrate carotid artery displacements under head movements.

FIGS. 40a-40d illustrate detection of a moving artery using the linear array probe.

FIGS. 41a-41d illustrate M-mode images collected by one sensing channel with increasing yawing rates.

FIG. 42 shows recorded pulse waveforms under increasing yawing rates from 0°/s to 80°/s.

FIGS. 43a-43b quantify the domain distance and visualization of the domain distributions.

FIG. 44 shows a heatmap of the classification accuracy observed after domain adaptation with different numbers of images from the target and source domains.

FIGS. 45a-45b show representative pressure waveforms recorded during cycling and HIIT.

FIGS. 46a-46b show measurements of the AIx.

FIGS. 47a-48b show measurements of the arterial stiffness index (β) before, during, and after exercise.

FIGS. 48a-48b show muscle recruitments and corresponding AIx during cycling and HIIT.

FIG. 49 shows an estimation of the stroke volume by the pulse contour method.

FIGS. 50a-50d illustrate acquisition errors in conventional ultrasonography.

FIG. 51 is a flowchart illustrating one example of a method for monitoring a physiologic parameter that may be performed by various embodiments of the USoP described herein.

### DETAILED DESCRIPTION

Described herein is a fully integrated autonomous ultrasonic-system-on-patch (USoP). The USoP integrates the ultrasonic probe and miniaturized wireless control electronics in a soft, wearable format, which overcomes the above-mentioned limitations. Multiple channels of deep tissue signals acquired from the subject are conditioned and preprocessed on-board, then wirelessly transferred to a backend receiver, where they are analyzed by a customized machine learning algorithm. When the USoP on the skin moves relative to the target tissue, the algorithm classifies the data and selects the best channel in real time, yielding a continuous data stream from the target tissue. Therefore, this technology allows continuous monitoring of deep tissue signals during human motion. The fully integrated autonomous USoP eliminates the operator dependency of conventional ultrasonography, standardizes the data interpretation process, and therefore expands the accessibility of this powerful diagnostic tool in both inpatient and outpatient settings.

It should be noted that while in the illustrative embodiments the back-end receiver analyzes the channels using machine learning algorithms, more generally any suitable artificial intelligence algorithms and techniques may be employed. Moreover, while in the illustrative embodiments this analysis is performed by a back-end receiver external to USoP, in other embodiments this analysis may be performed on the USoP itself by, e.g., the control electronics integrated therewith.

### Design of the USoP

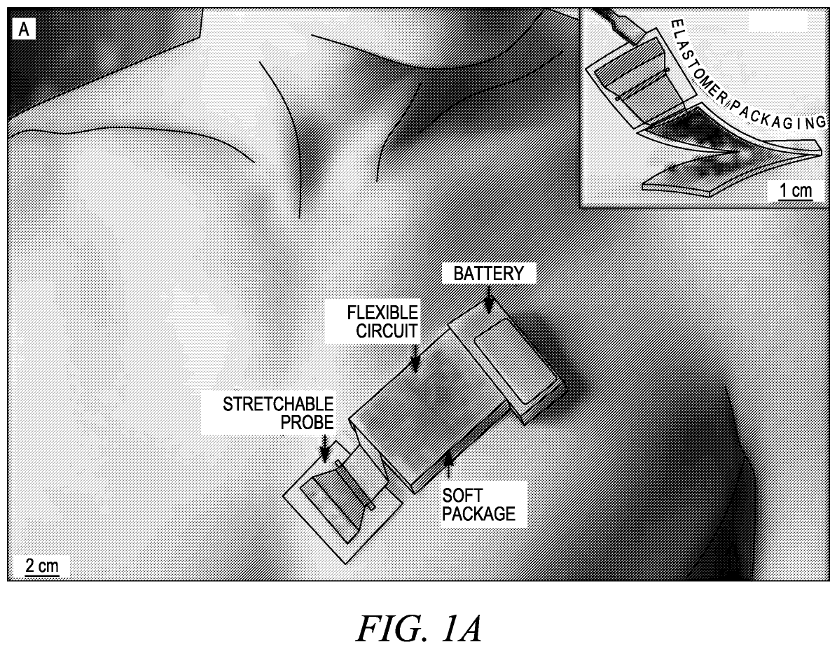

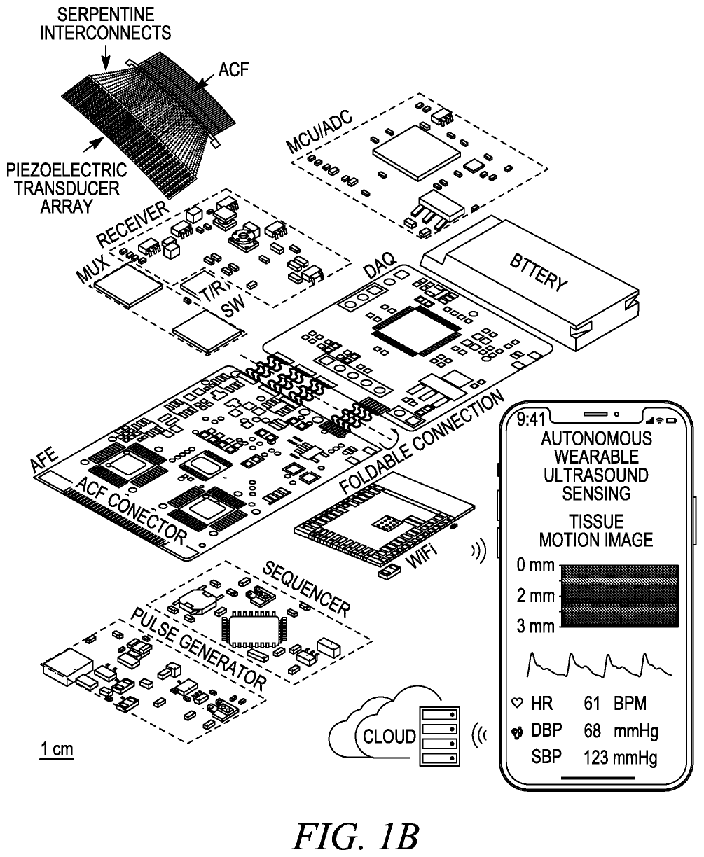

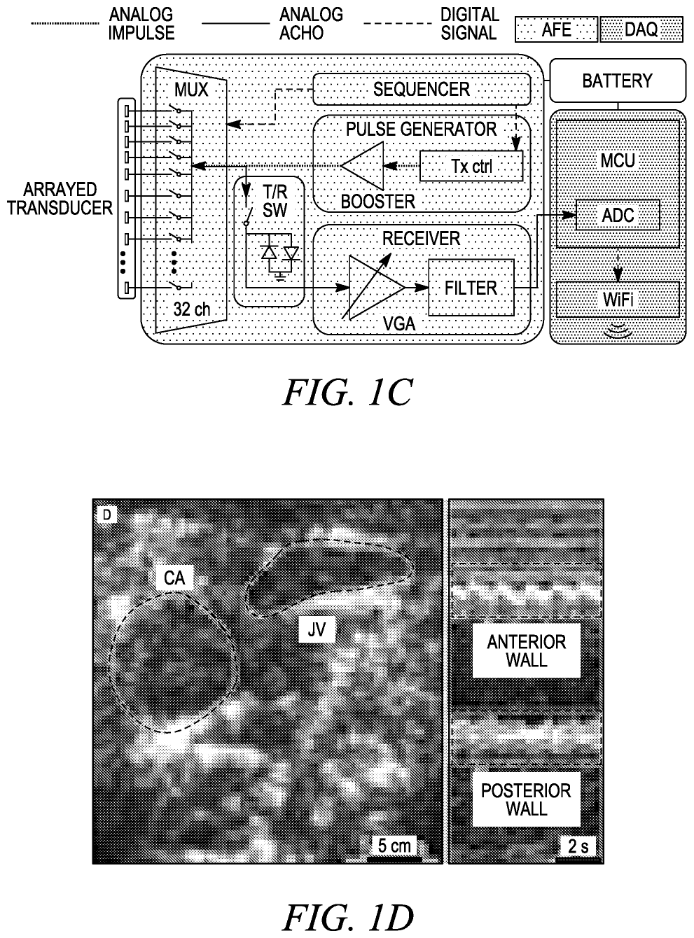

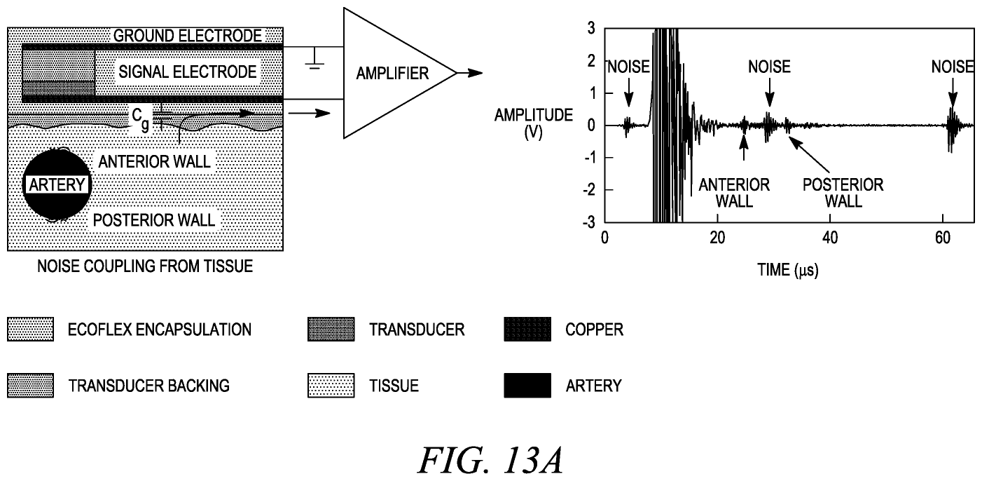

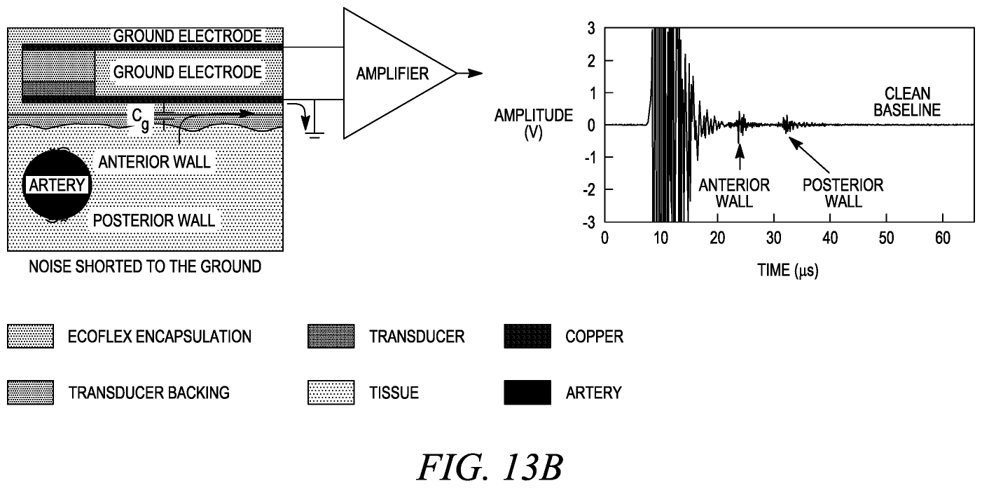

In one embodiment, the USoP hardware consists of an ultrasound probe and control electronics which are fabricated in a miniaturized, soft format (FIG. 1a). The ultrasonic probe is made of piezoelectric transducers, backing materials, serpentine interconnects, and contact pads, similar to our reported structures, illustrative examples of which are shown in the following references which are hereby incorporated by reference in their entirety: Wang, C. et al. Monitoring of the central blood pressure waveform via a conformal ultrasonic device. Nat. Biomed. Eng. 2, 687-695 (2018); Wang, C. et al. Continuous monitoring of deep-tissue haemodynamics with stretchable ultrasonic phased arrays. Nat. Biomed. Eng. 5, 749-758 (2021); and Hu, H. et al. A wearable cardiac ultrasound imager. Nature 613, 667-675 (2023). This soft probe design reduced noise coupling, enhanced resolution, enabled gel-free acoustic coupling, and ensured probe durability (see FIGS. 13-17 and the section below entitled Ultrasonic probe fabrication and layout designs).

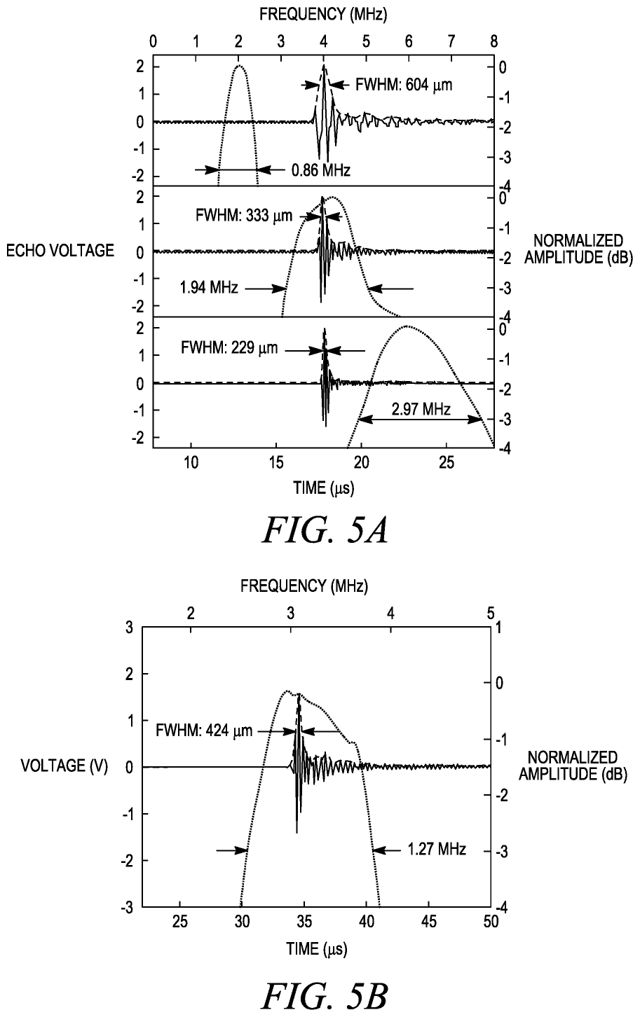

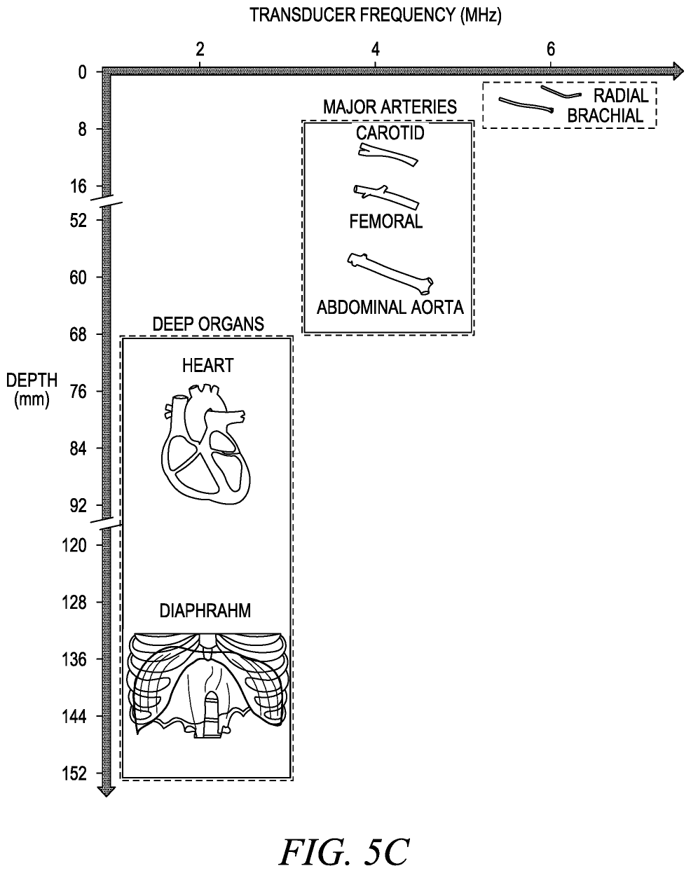

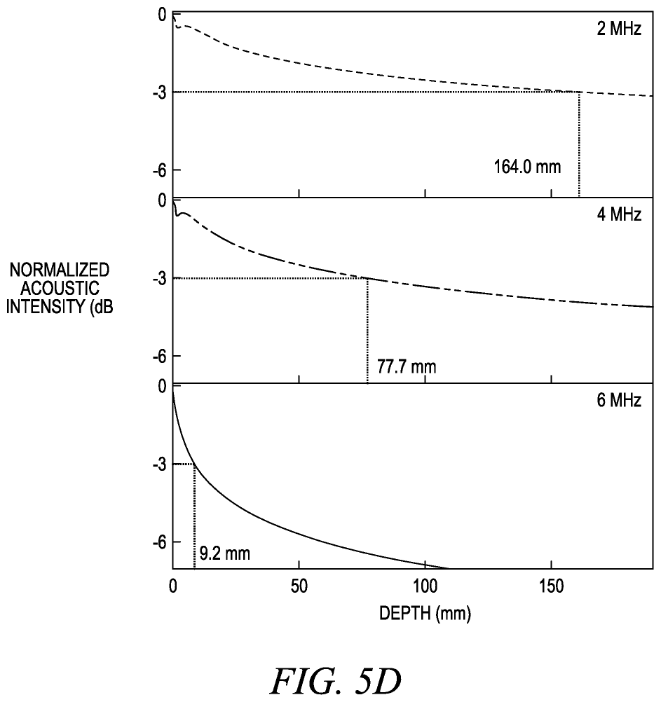

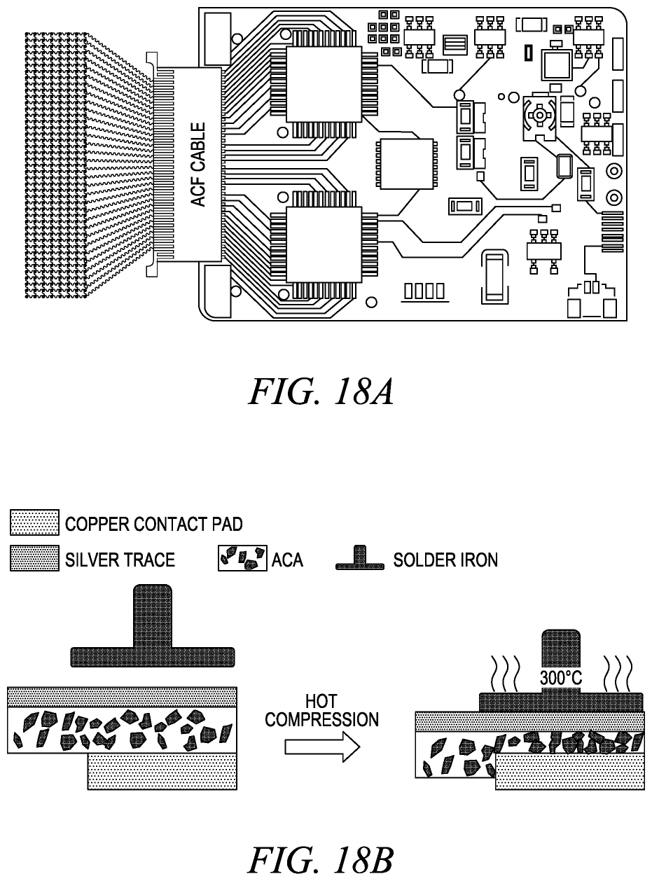

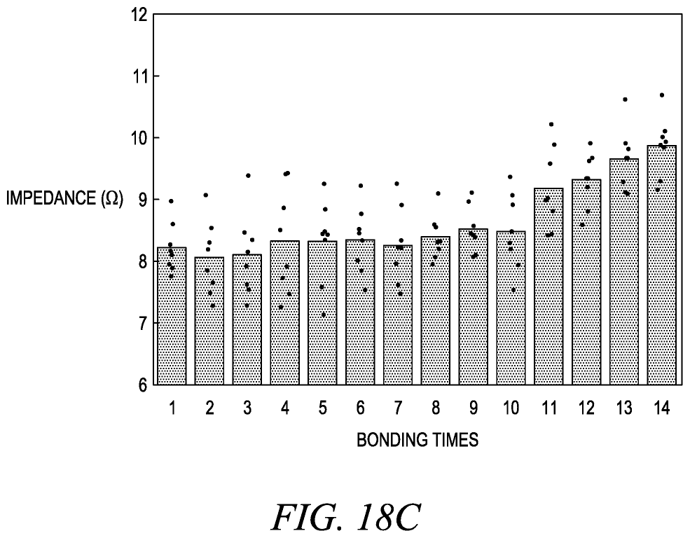

In an illustrative embodiment, we design the probes of center frequencies from 2 MHz to 6 MHz to achieve the desired bandwidth, axial resolution, and penetration. We determine the bandwidth as the −3 dB frequency band of the pulse-echo response, spatial resolution as the full width at half maximum of the pulse-echo response, and penetration depth as the −3 dB attenuation point in tissues. All soft probes can achieve a relative bandwidth of ˜50%, which is similar to a commercial probe (FIG. 5). The 2 MHz transducers achieve a depth of ˜164 mm with an axial resolution of ˜600 μm for targeting visceral organs (e.g., heart and diaphragm). The 4 MHz transducers achieve a depth of ˜78 mm with an axial resolution of ˜330 μm for targeting major arteries (e.g., aorta, carotid, and femoral arteries). The 6 MHz transducers achieve a depth of ˜9 mm and an axial resolution of ˜230 μm for targeting smaller peripheral arteries (e.g., radial and brachial arteries) (FIG. 5). To achieve desired beam profiles, we customize three probe layouts: disc, linear array, and two-dimensional array, for penetrative, wide, and narrow beam, respectively (see FIG. 18 and the section below entitled Ultrasonic probe fabrication and layout designs). For electrical connection, we use anisotropic conductive films with easy attachment and detachment for repetitive use (FIG. 18).

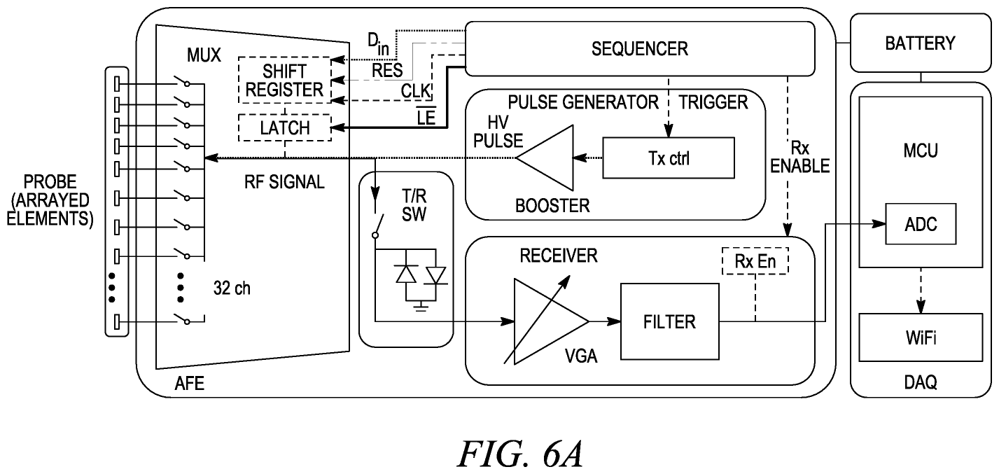

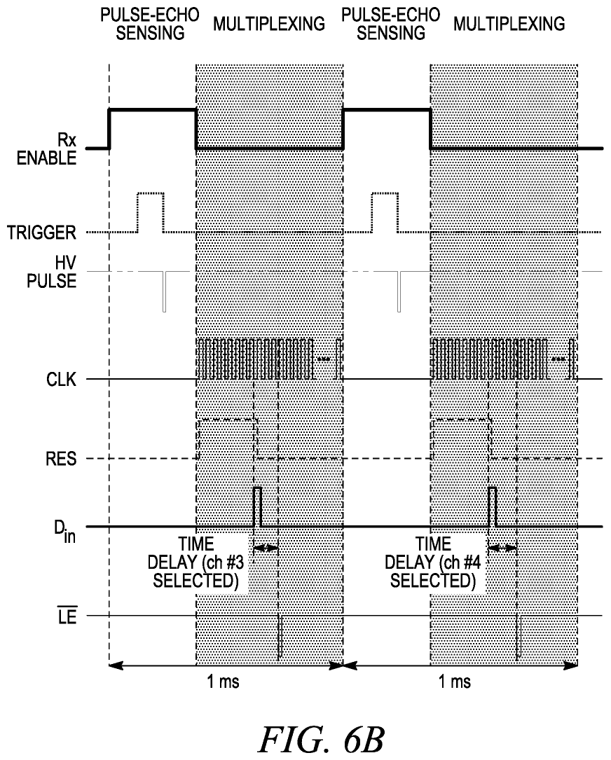

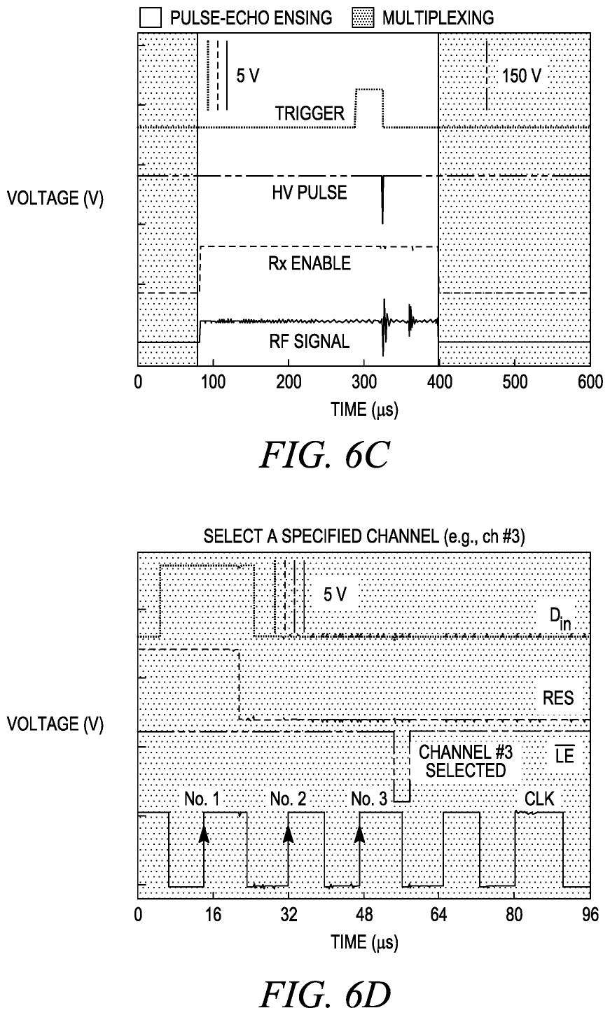

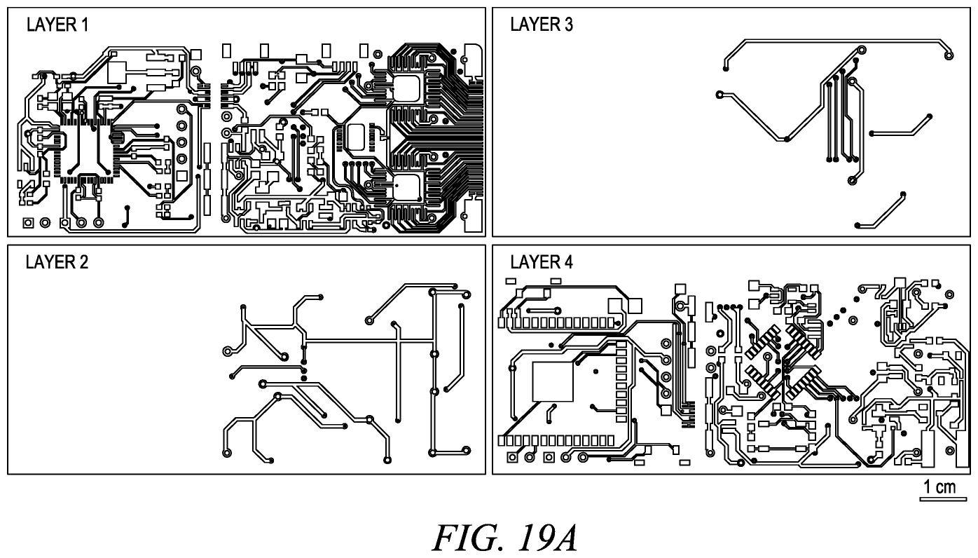

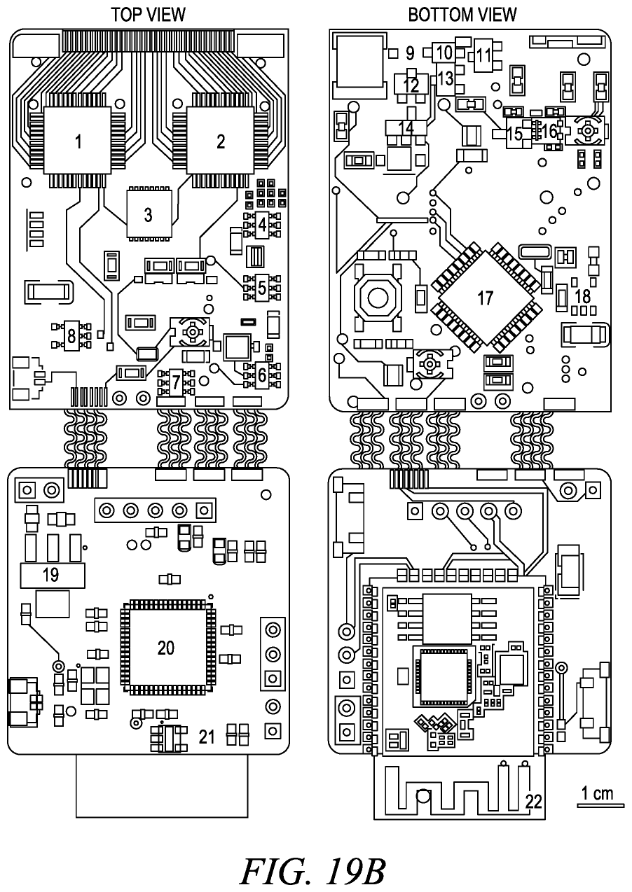

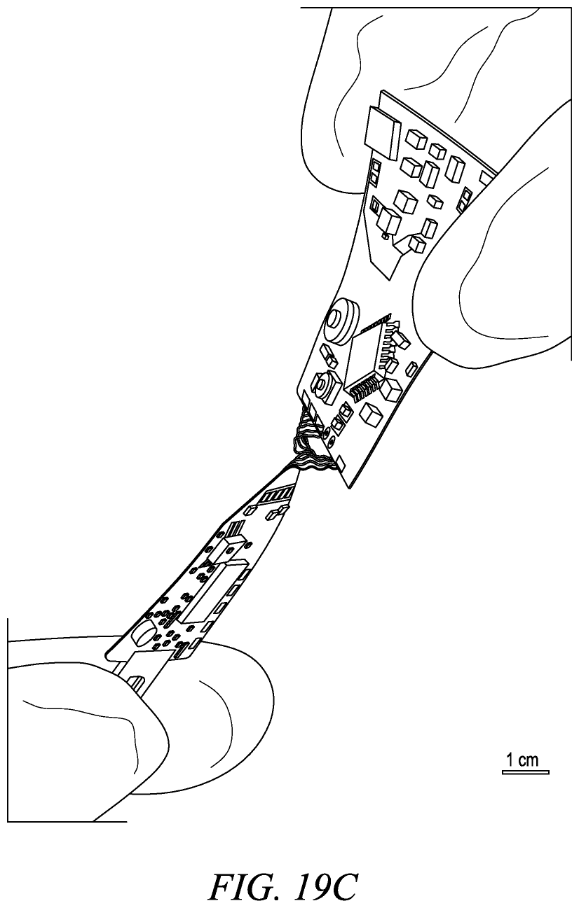

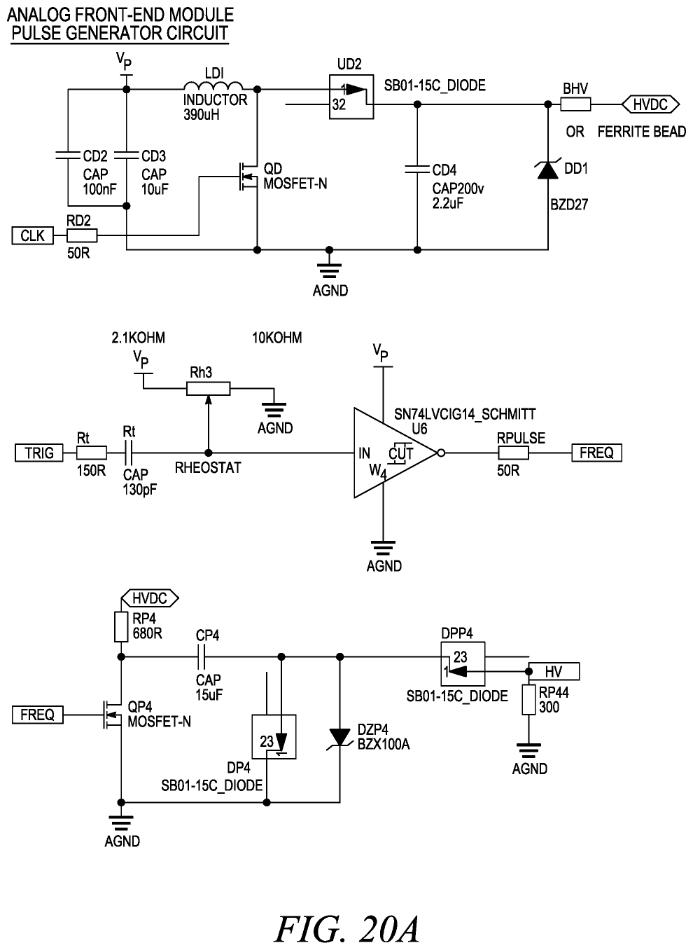

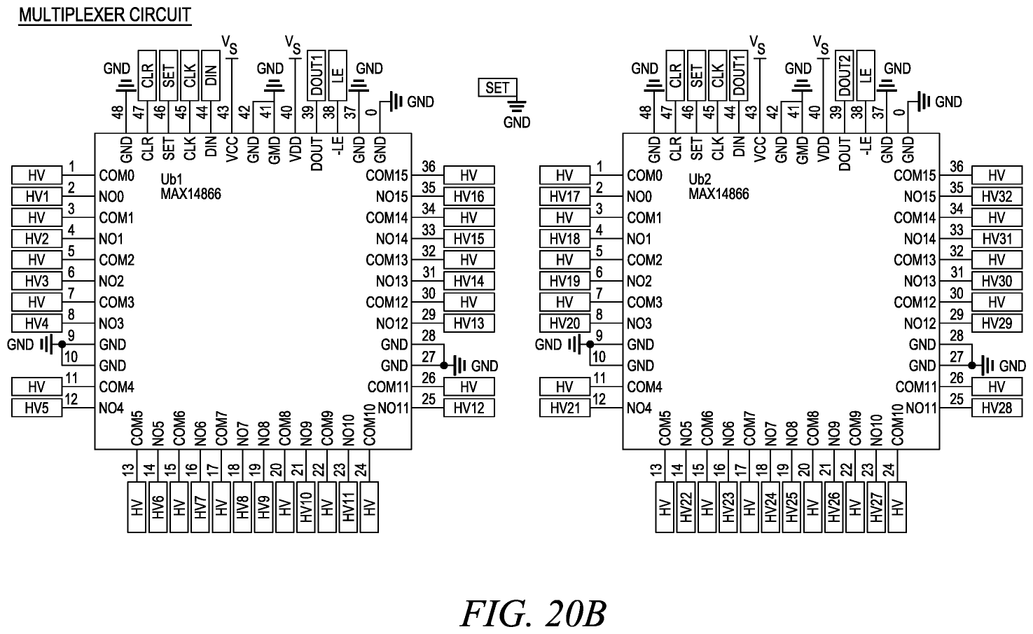

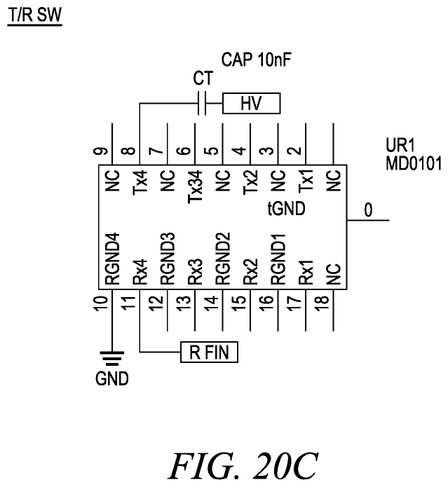

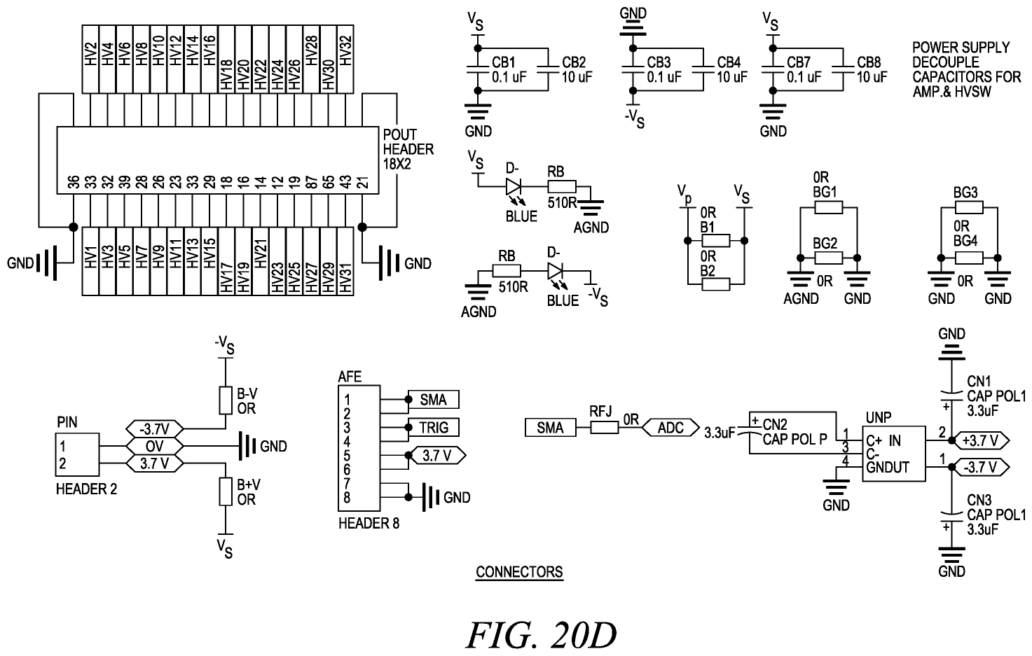

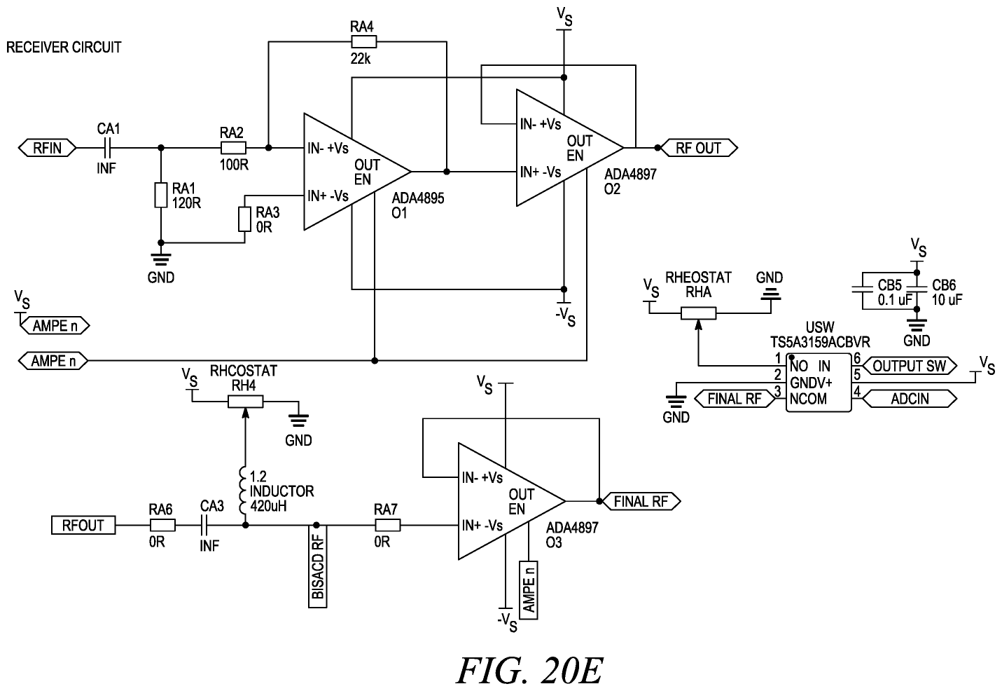

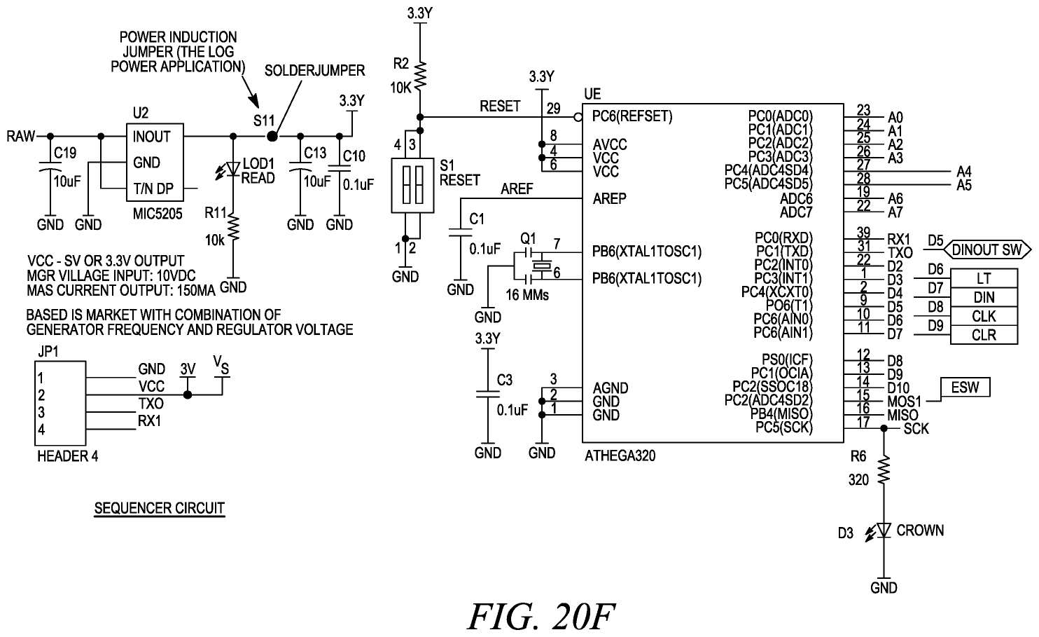

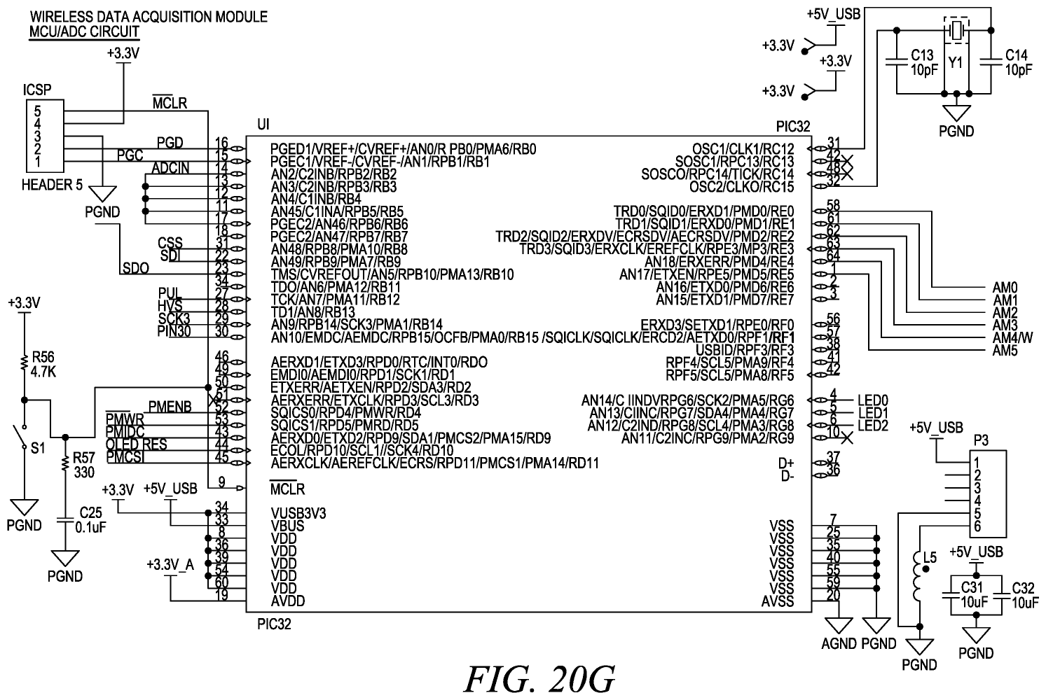

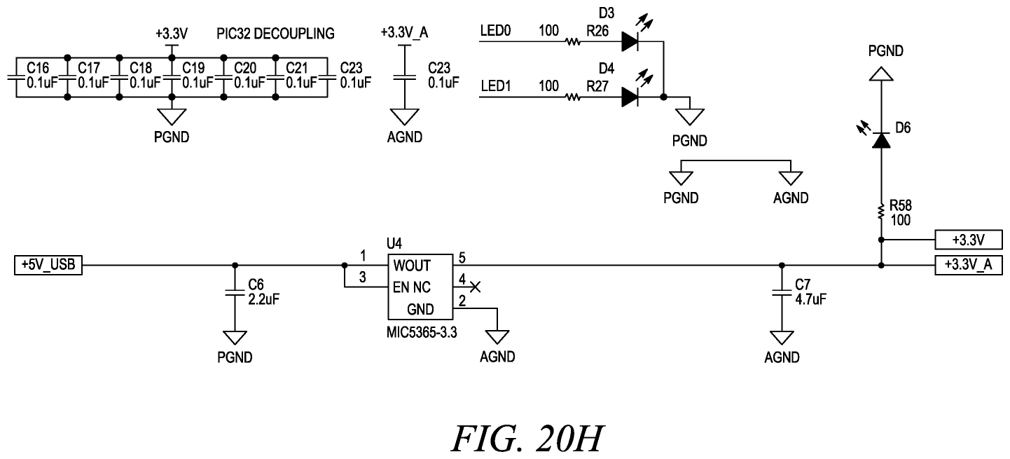

In an illustrative embodiment, the control electronics are designed as a flexible printed circuit board (FIG. 19 and Table 2) for ultrasonic sensing and wireless communication. The circuitry includes an analog front-end (AFE) and a data acquisition (DAQ) module (FIG. 1b and FIG. 20). The AFE achieves ultrasonic sensing through coordinated sequence control of multiple components (see FIG. 6 and the section below entitled Sequence control of the ultrasonic sensing). First, the sequencer initiates sensing by sending trigger signals to the pulse generator and multiplexer. Then, the pulse generator reads the trigger signals and outputs high-voltage impulses to activate the ultrasound transducers. Meanwhile, the multiplexer drives the arrayed transducers to generate ultrasound and receive echoes. Finally, the echoes are collected by the transmit/receive switch, and then amplified and filtered by the receiver circuit. After the AFE completes the ultrasonic sensing process, the analog echoes are relayed to the DAQ module. The microcontroller unit (MCU) samples the echoes with a built-in analog-to-digital converter, and then the Wi-Fi module wirelessly transmits the digitalized echoes to a terminal device (e.g., a smartphone or a computer), where an online machine learning algorithm and an application program can process and display the signals autonomously (FIG. 1c).

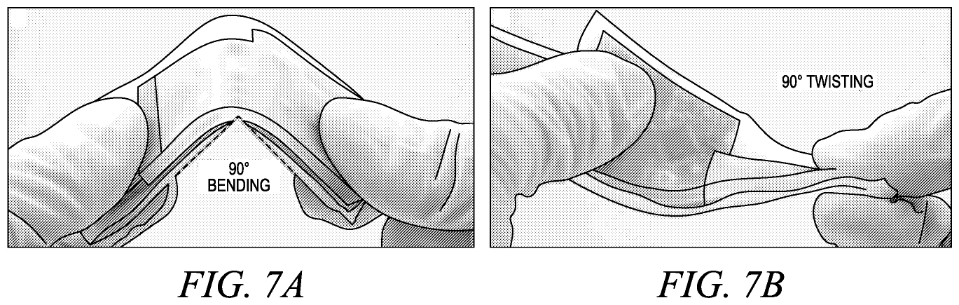

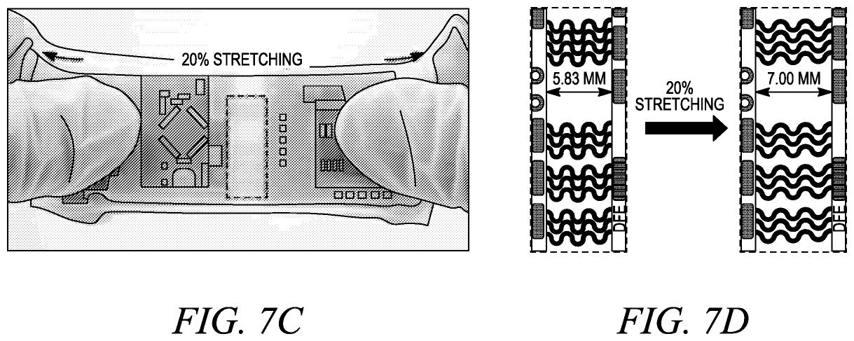

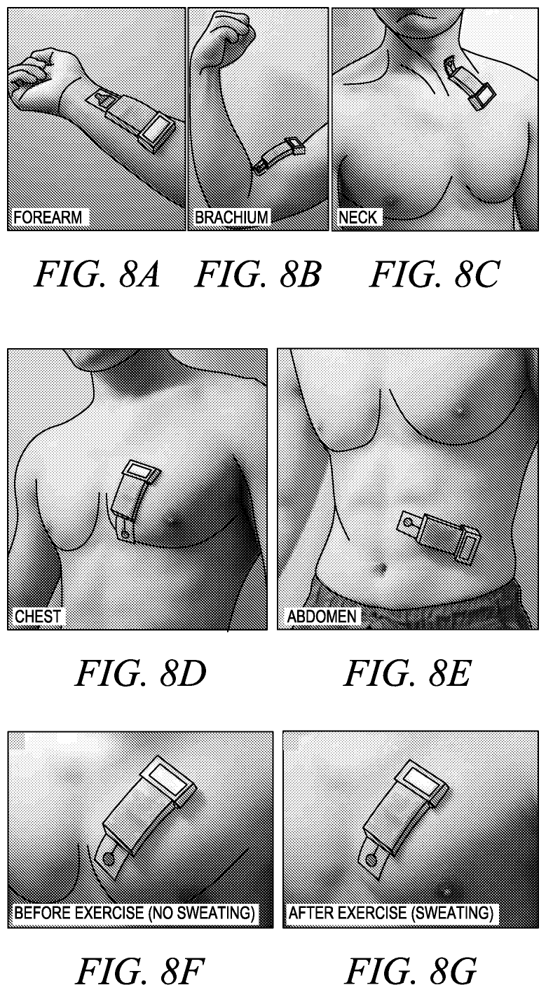

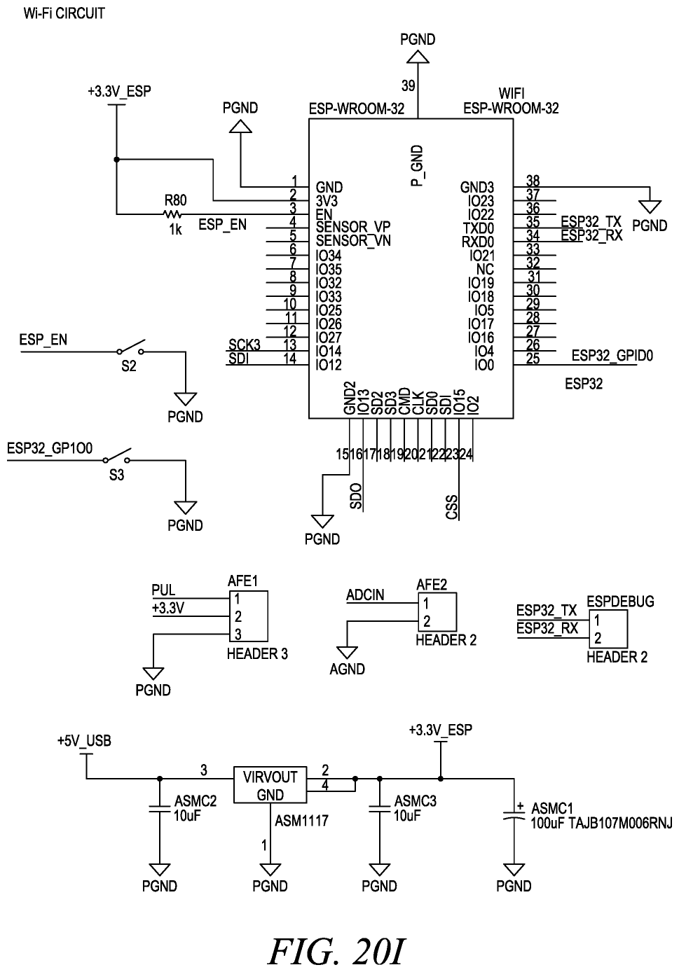

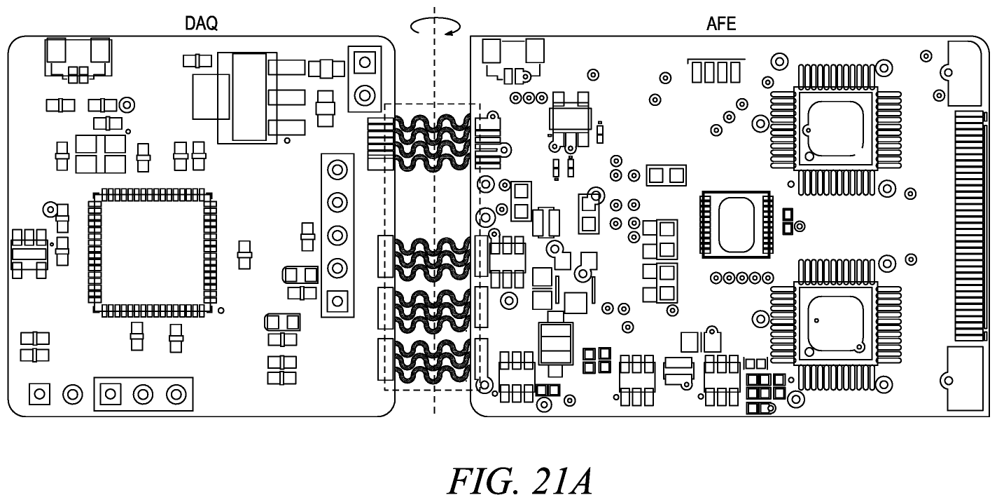

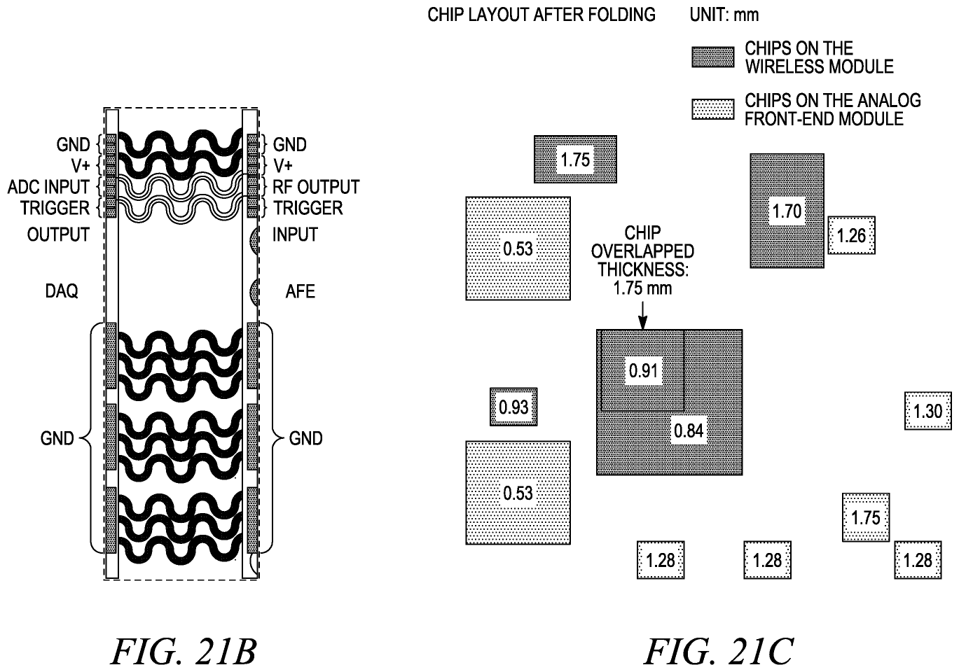

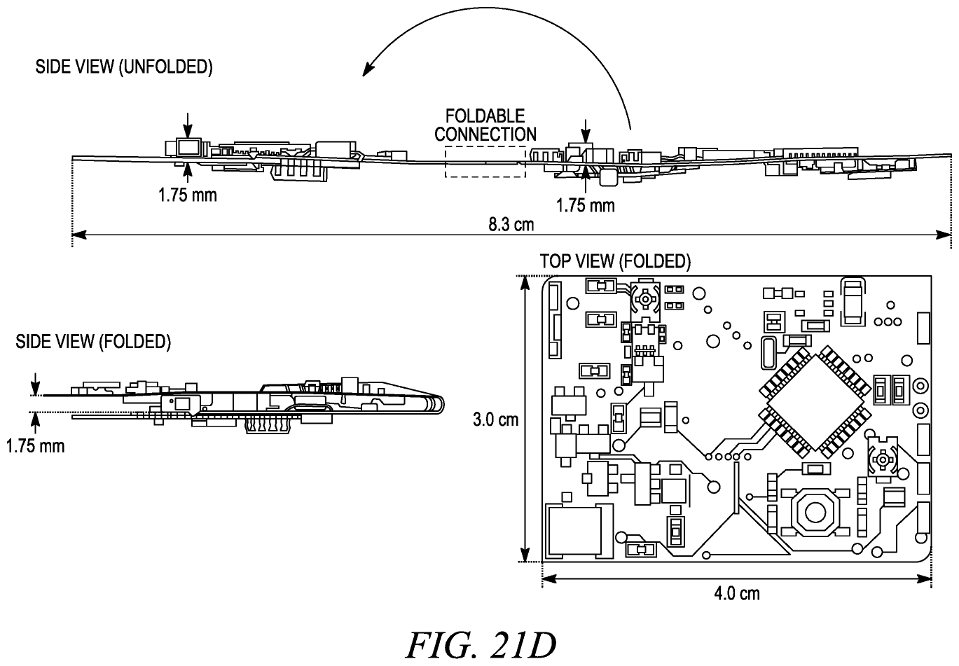

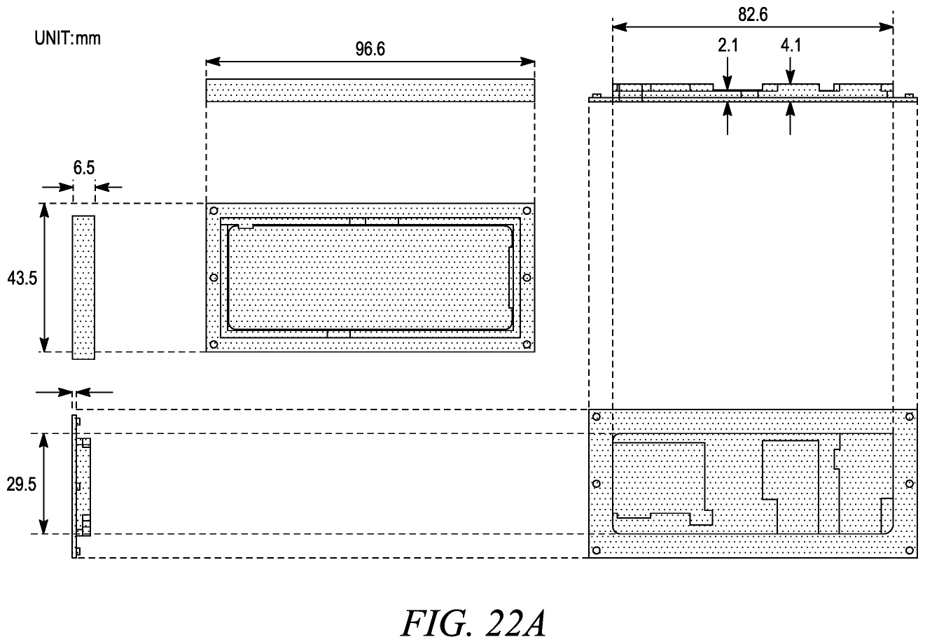

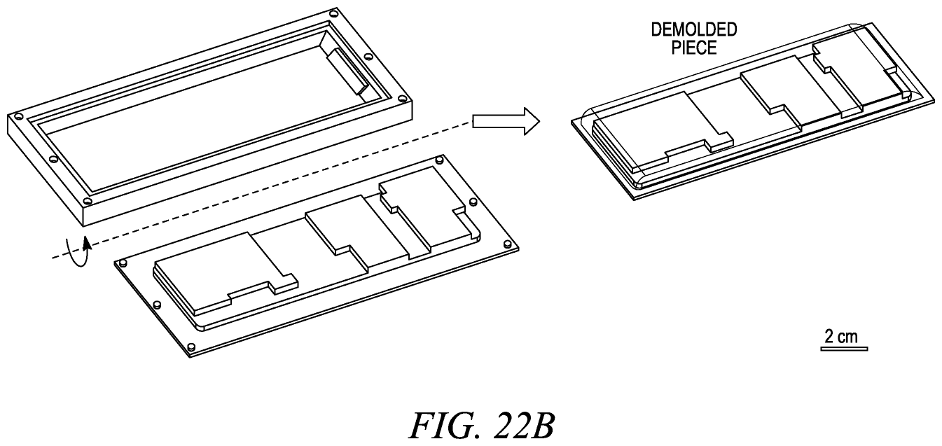

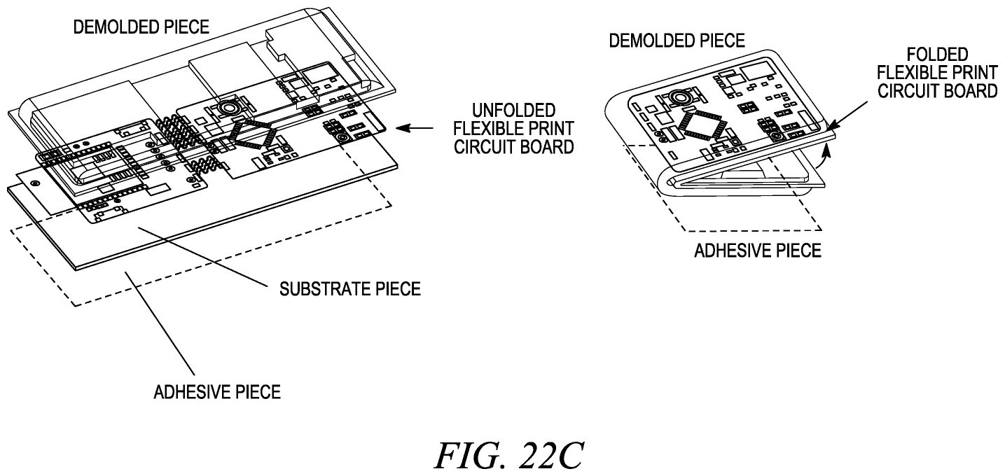

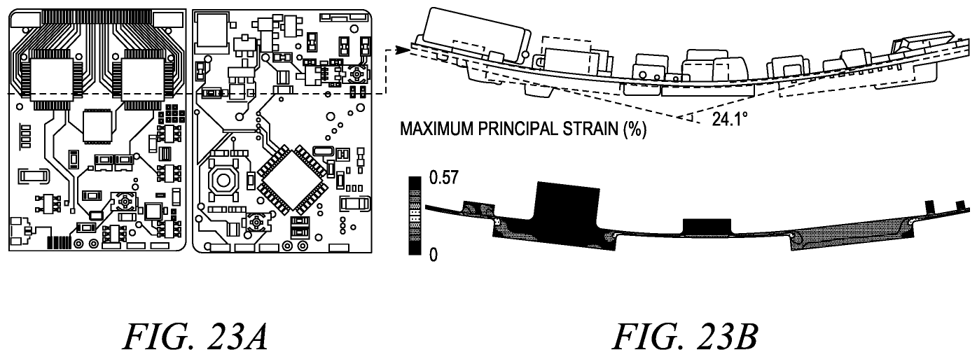

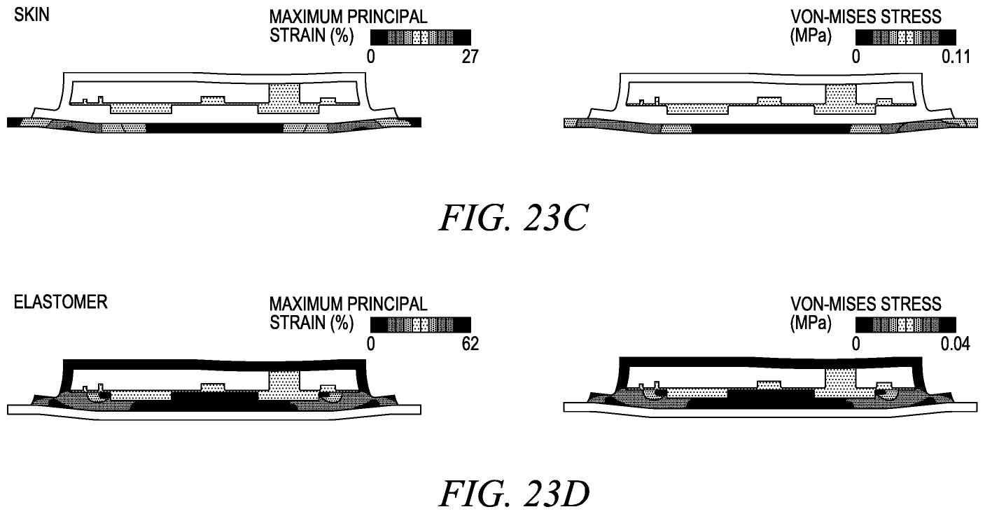

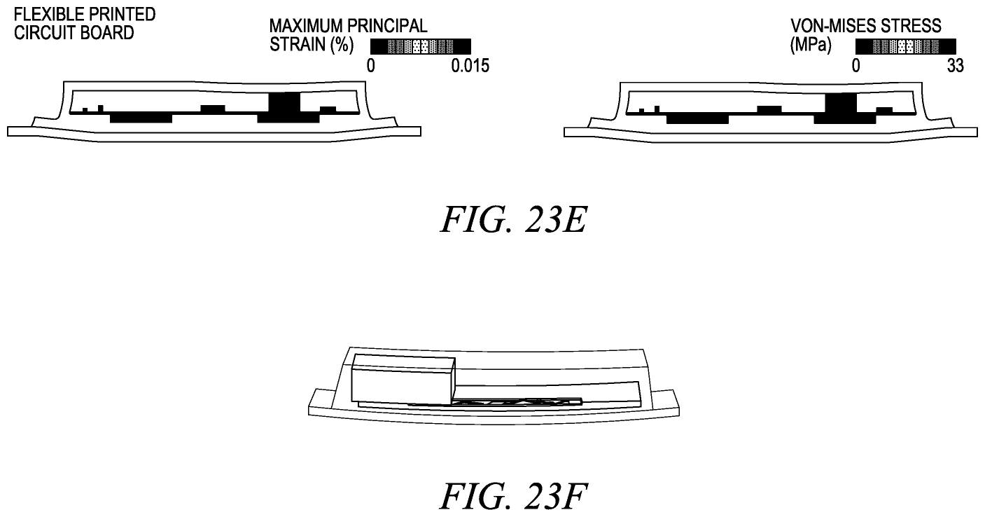

The AFE and the DAQ modules are interconnected by serpentine wires that allow for folding to minimize their footprint (FIG. 21). An elastomeric encapsulation mitigates strain concentrations and protects the circuit from irreversible deformations (FIGS. 22-23 and Methods). The fully integrated system can be bent, stretched, and twisted (FIG. 7) and be conformally laminated on the human body (FIG. 1a, FIG. 8).

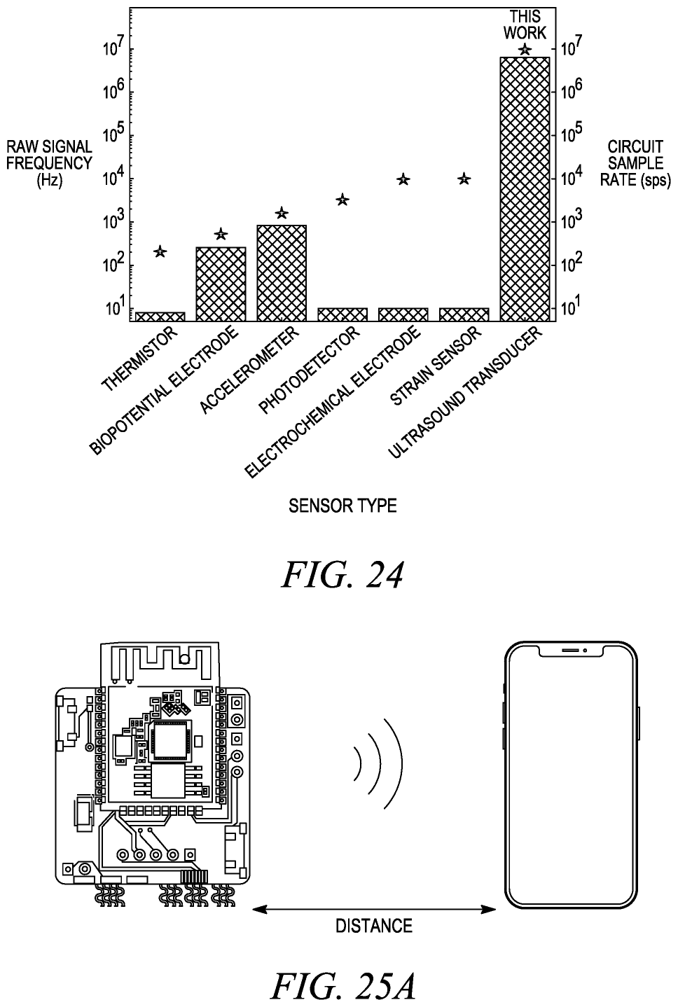

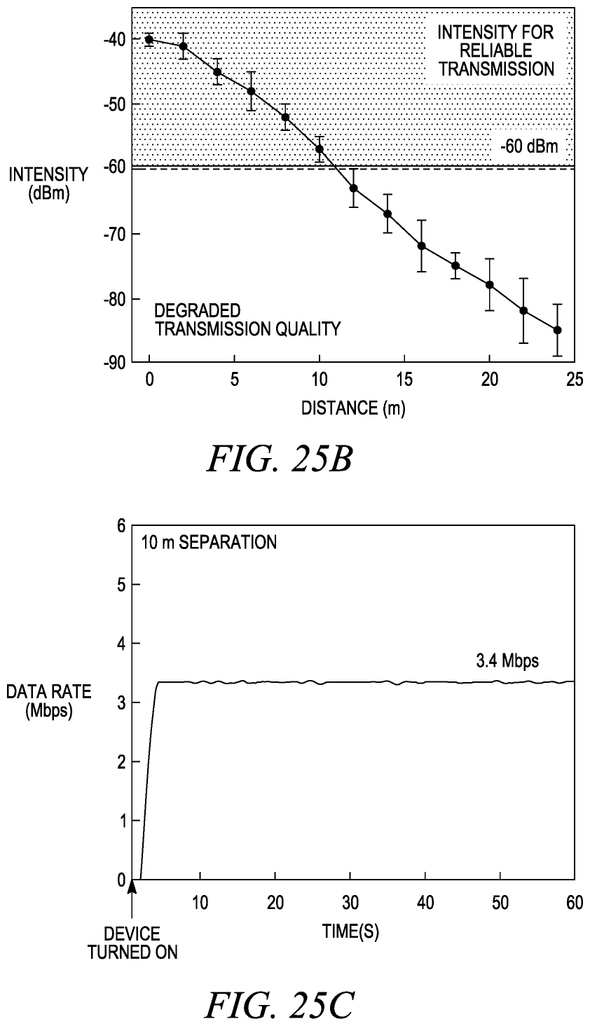

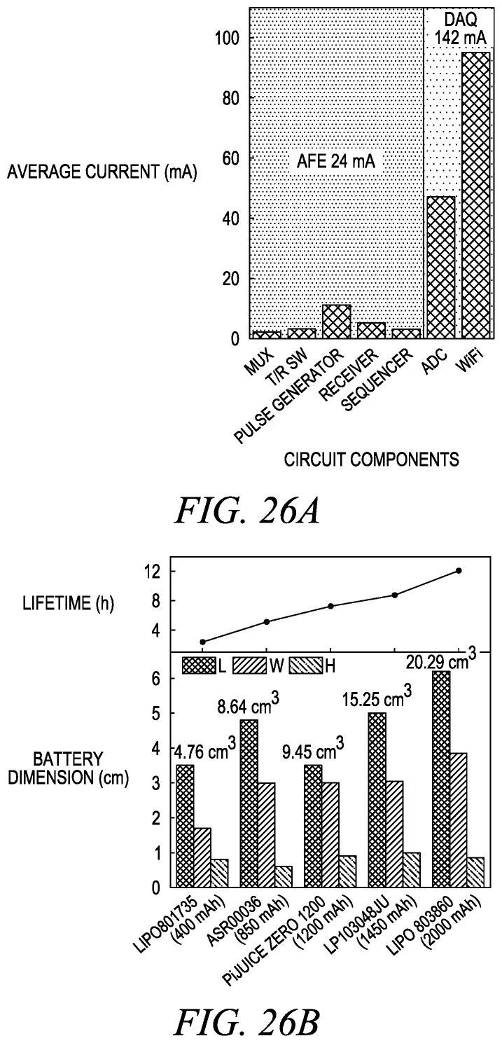

The ultrasonic probes have MHz-level bandwidth, significantly higher than other common sensors (FIG. 24). Therefore, achieving high sensing bandwidths and sampling rates is critical for the circuitry design. In this work, the DAQ has a sampling rate of 12 Msps corresponding to a sensing bandwidth of 6 MHz. The Wi-Fi module can transmit such wide-band signals at a distance of ˜10 m and a speed of 3.4 Mbps with zero data loss (FIG. 25). The USoP system has a power consumption of ˜614 mW. A standard 3.7 V commercial lithium-polymer battery can enable continuous operation for up to 12 hours (FIG. 26).

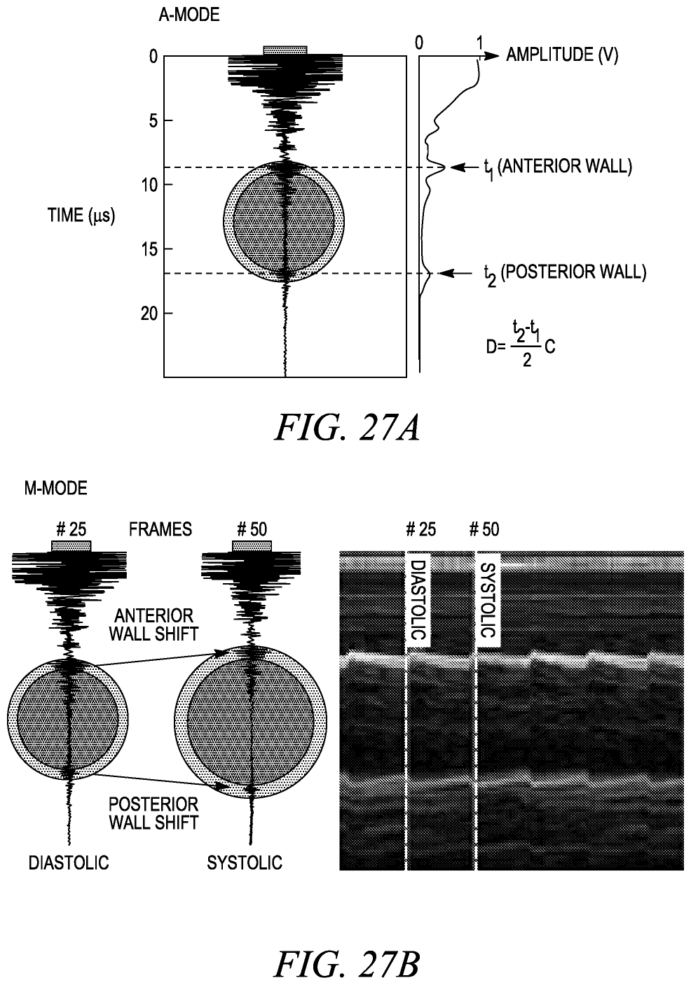

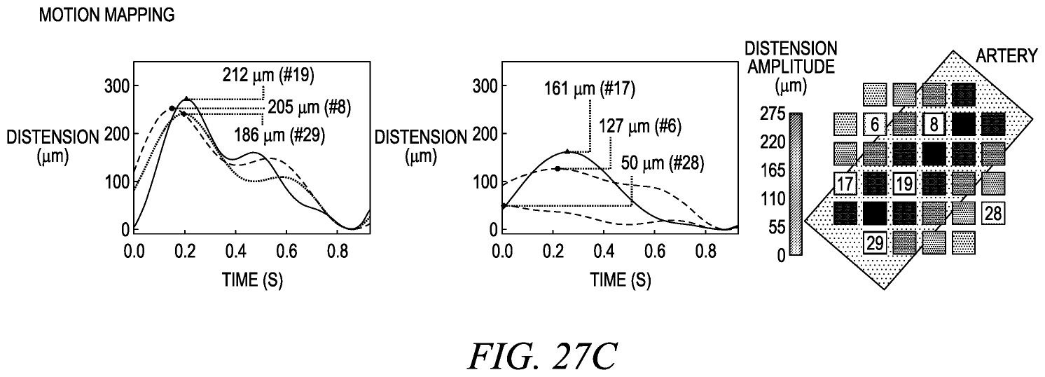

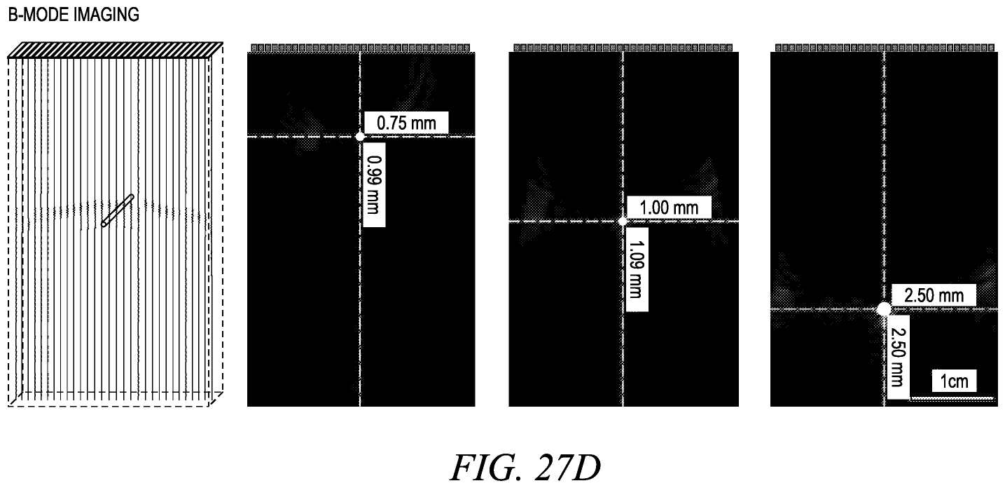

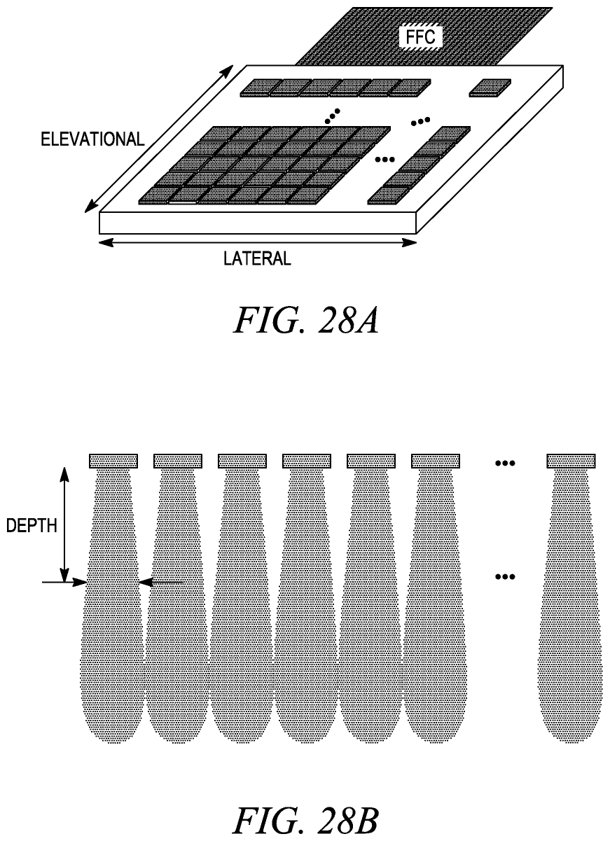

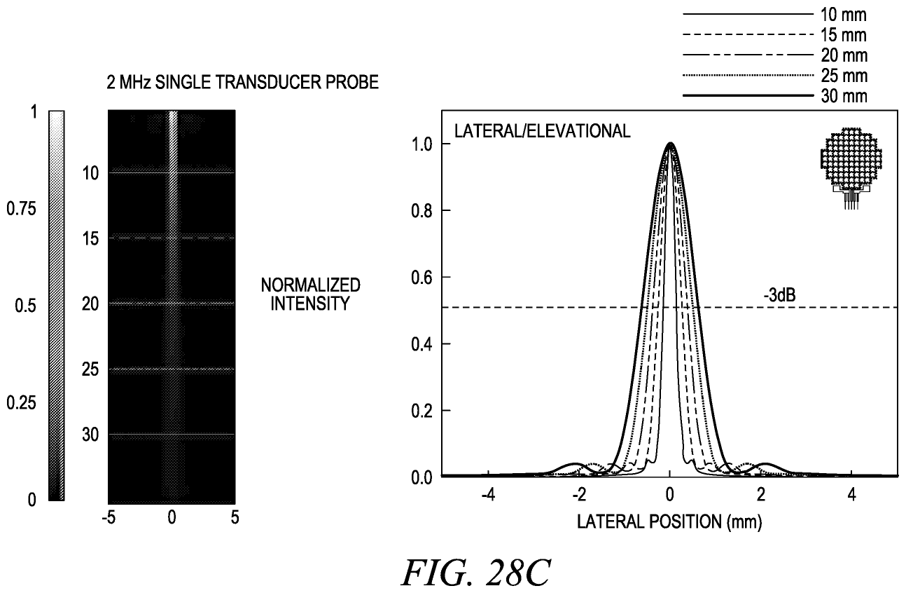

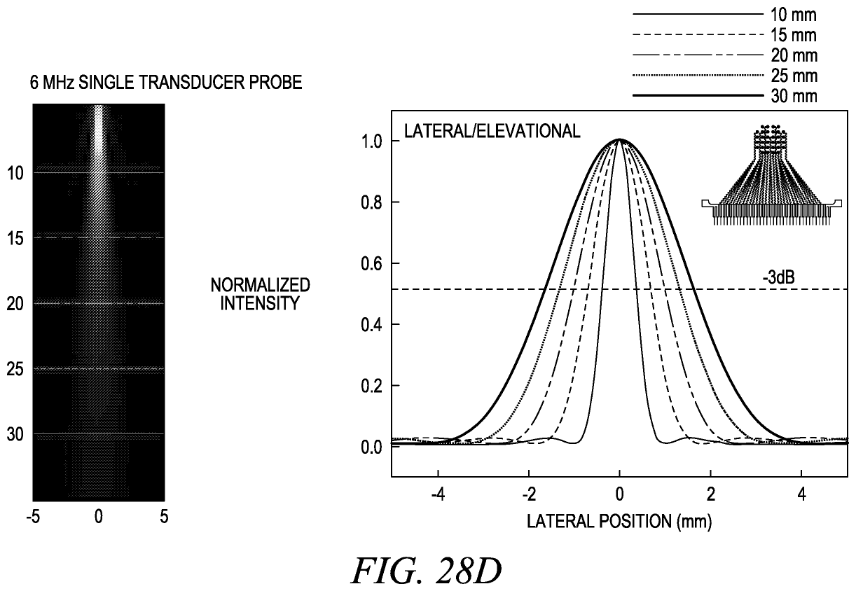

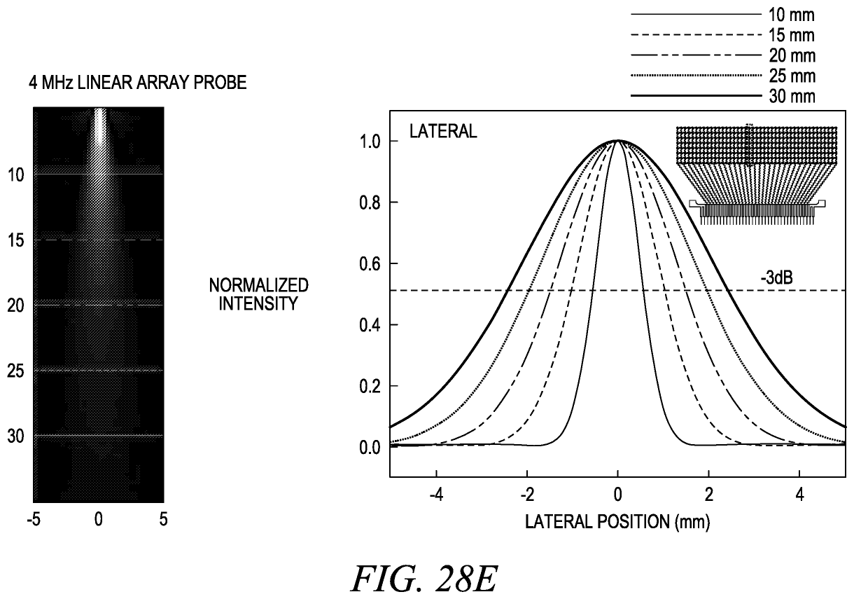

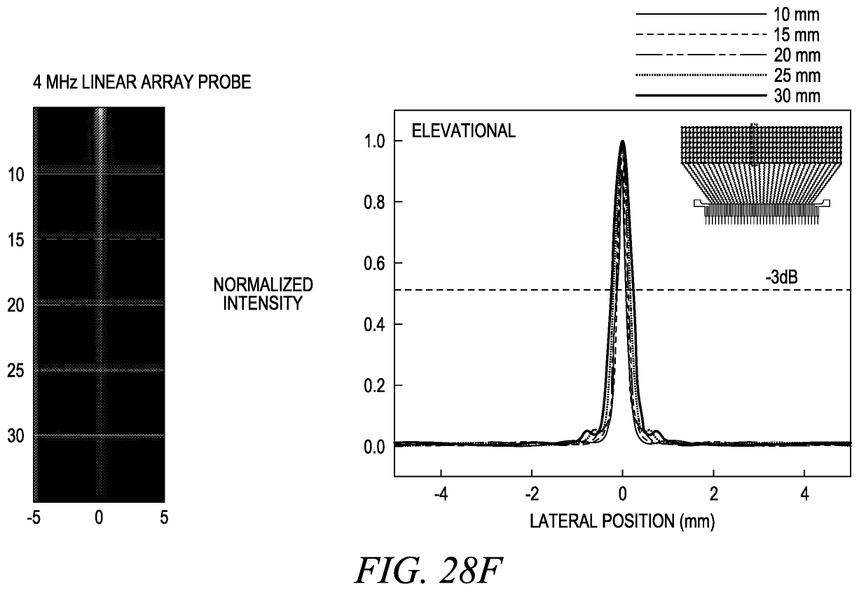

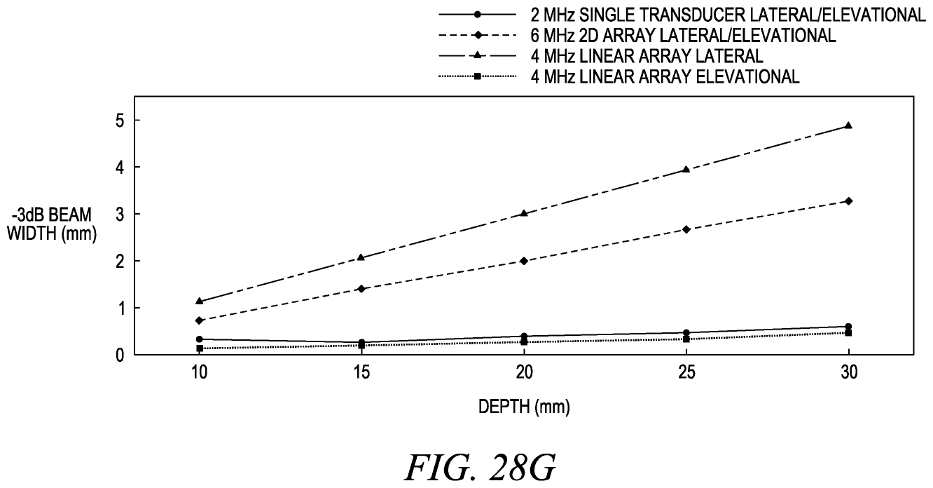

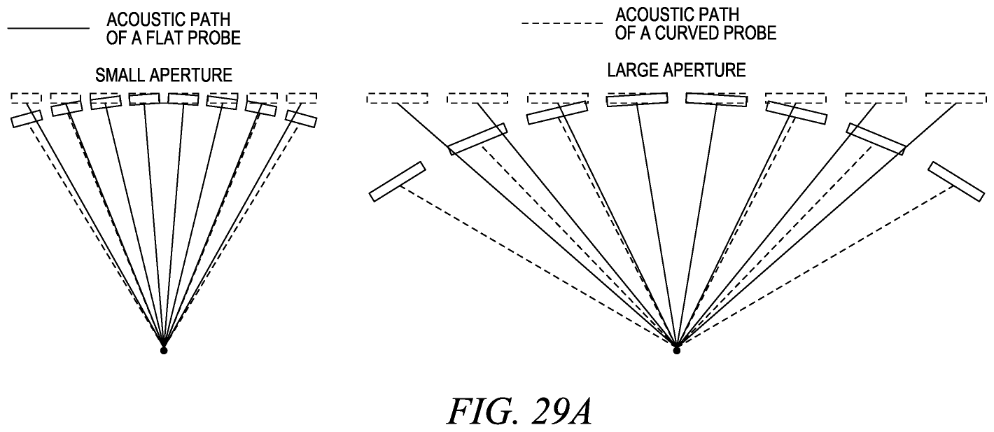

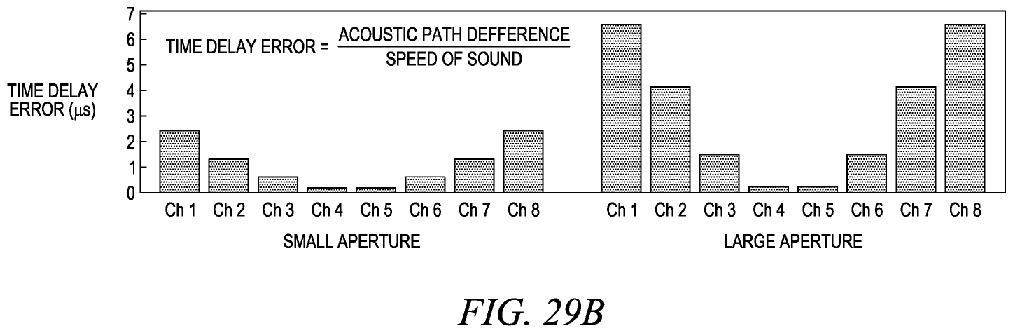

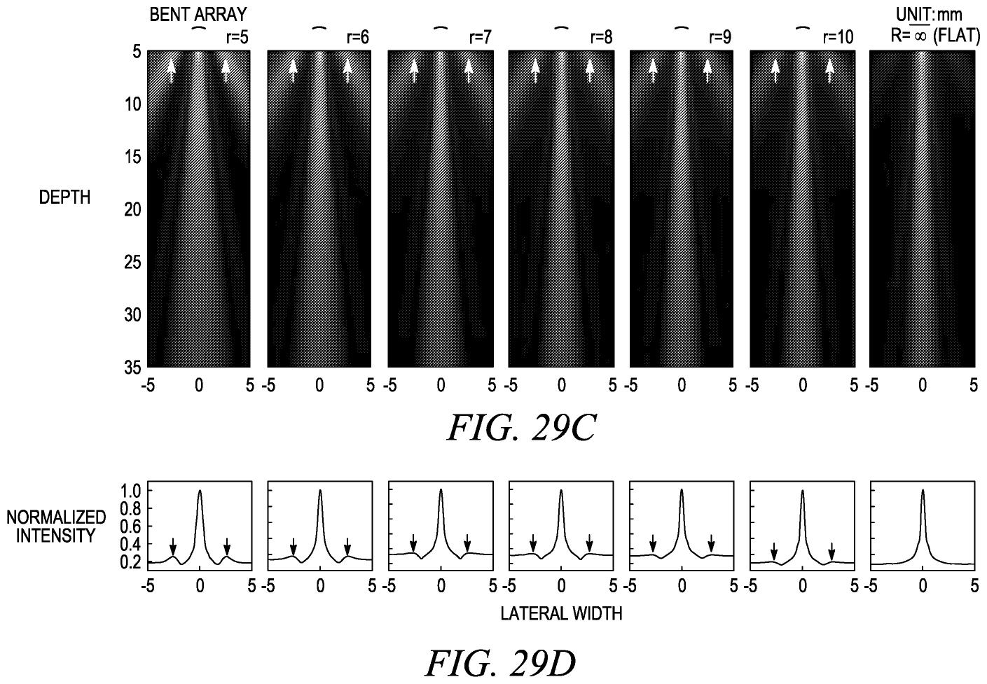

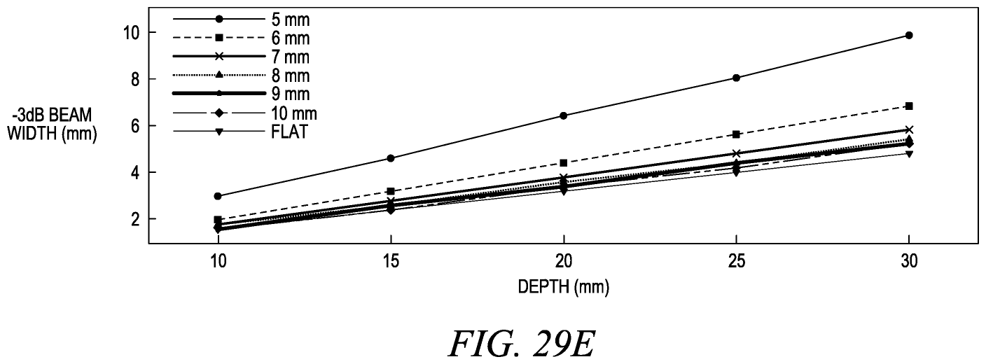

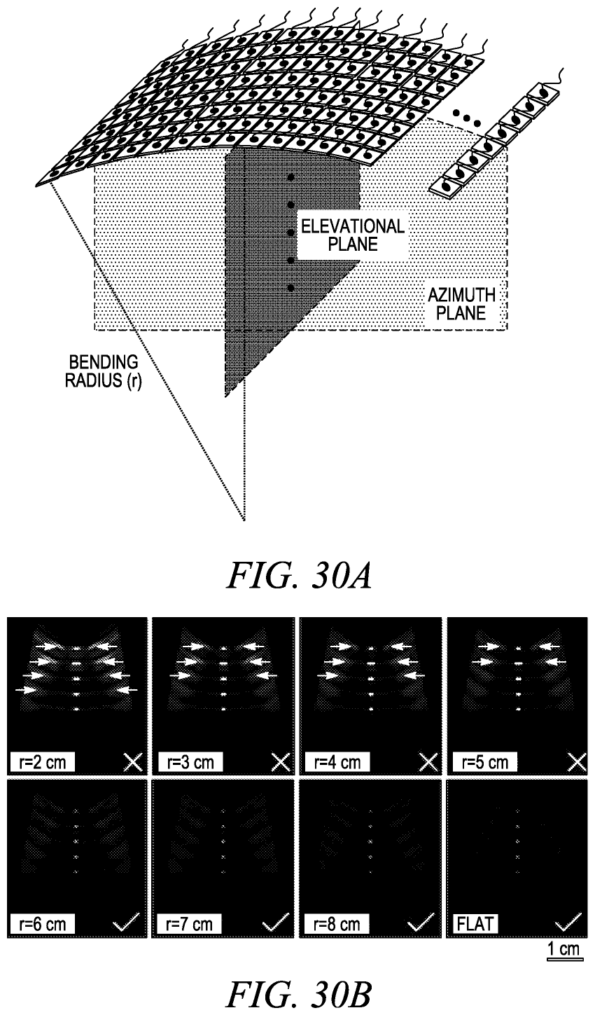

The USoP can perform tissue sensing in multiple modalities, including amplitude mode (A-mode), brightness mode (B-mode), and motion mode (M-mode), to reveal the tissue structures and interface movements (see FIG. 27, the section below entitled Multi-mode ultrasonic sensing, FIG. 1d). We characterized the elevational and lateral resolutions of these sensing modalities since they are of particular interest in deep tissue sensing. In A-mode and M-mode, the elevational and lateral resolutions show a degrading trend when the sensing depth increases (see FIG. 28 and the section below entitled Multi-mode ultrasonic sensing). In B-mode, the elevational resolution can be defined by the transmission beam pattern, while the lateral resolution can be determined directly from image reconstruction (see FIG. 27 and the section below entitled Multi-mode ultrasonic sensing). When the probes conform to skin surfaces within certain radius thresholds, the soft probes offer stability in sensing. For A-mode and M-mode, the resolutions can be maintained with an array bending radius >6 mm (see FIG. 29 and the section below entitled The sensing stability under probe deformation). For B-mode, the image artifacts could be neglected when the bending radius of the array >6 cm (see FIG. 30 and the section below entitled The sensing stability under probe deformation).

### Physiological Signal Recording and Validation

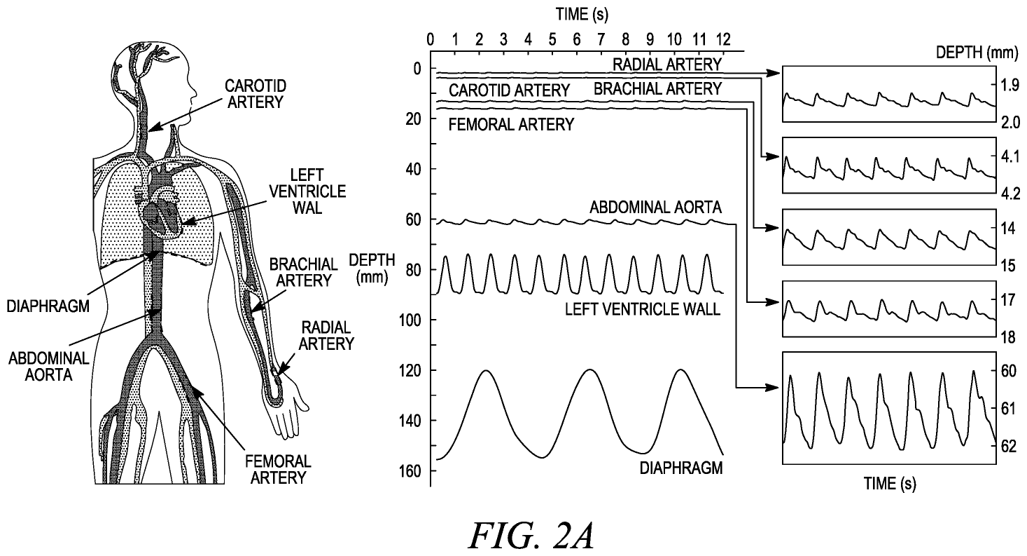

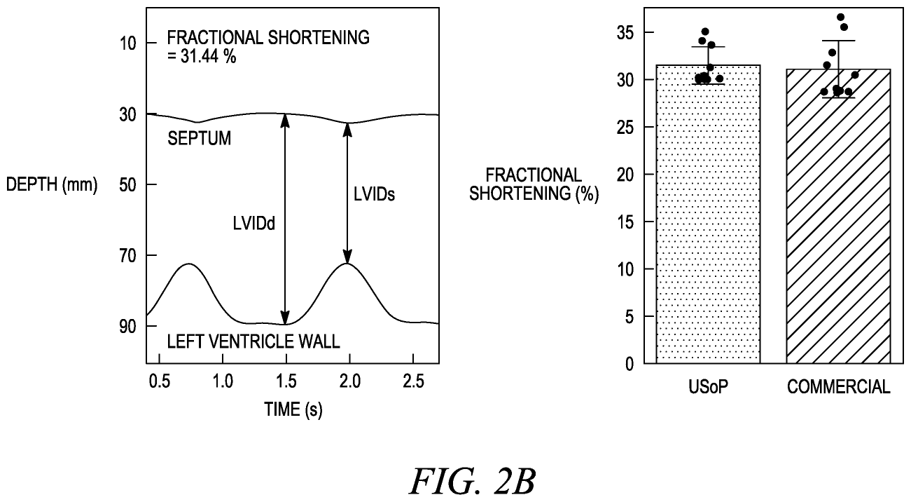

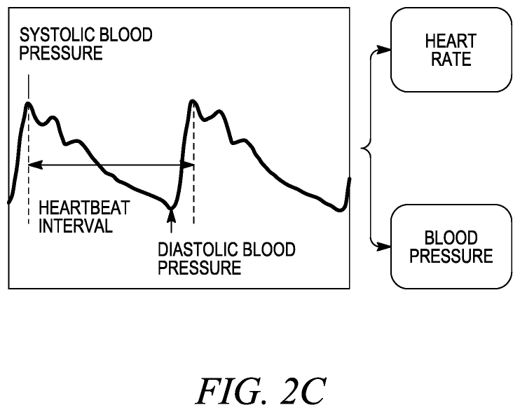

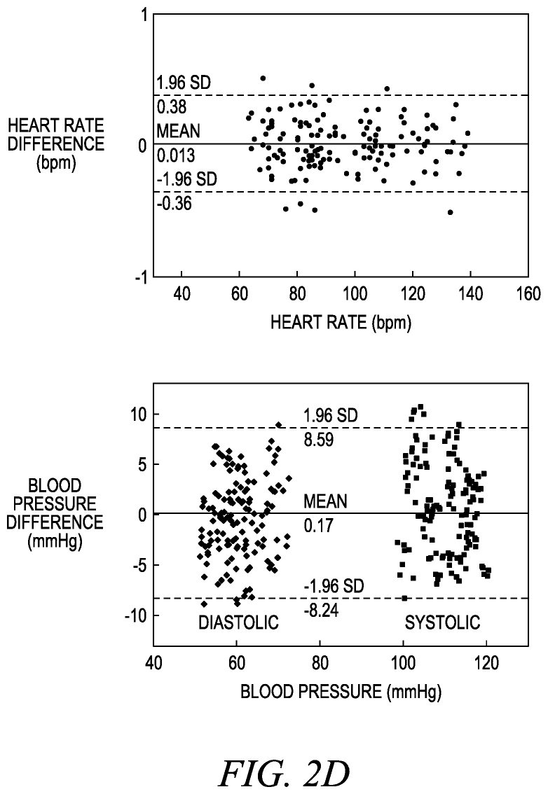

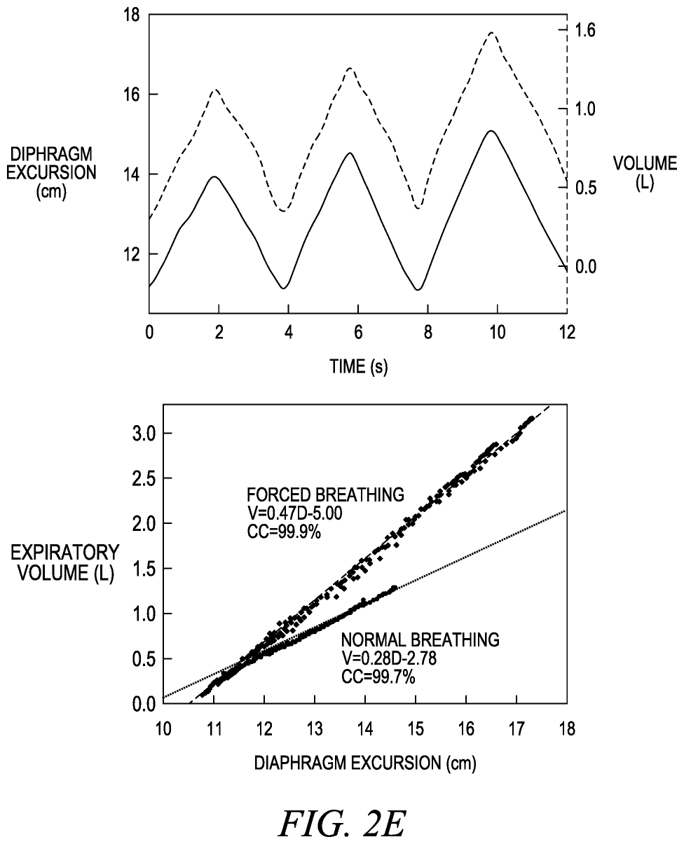

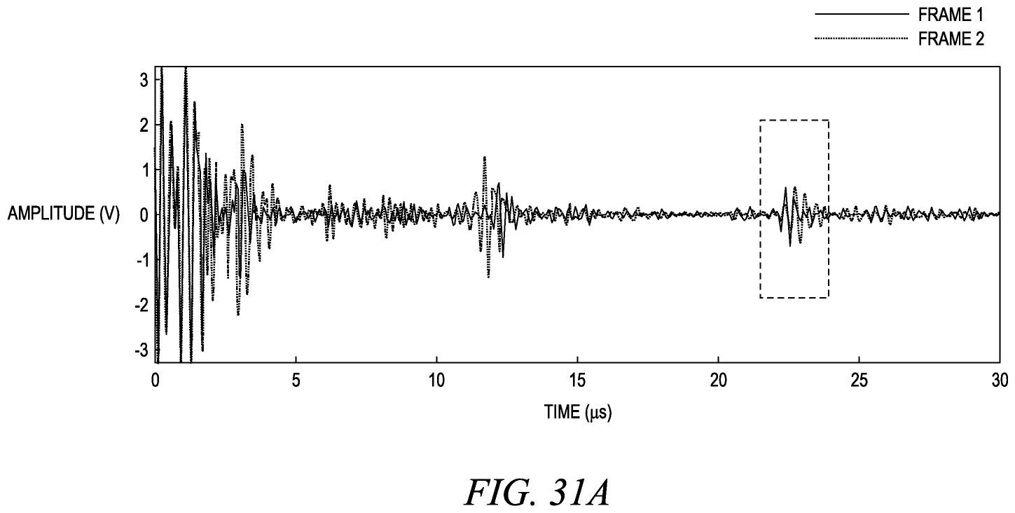

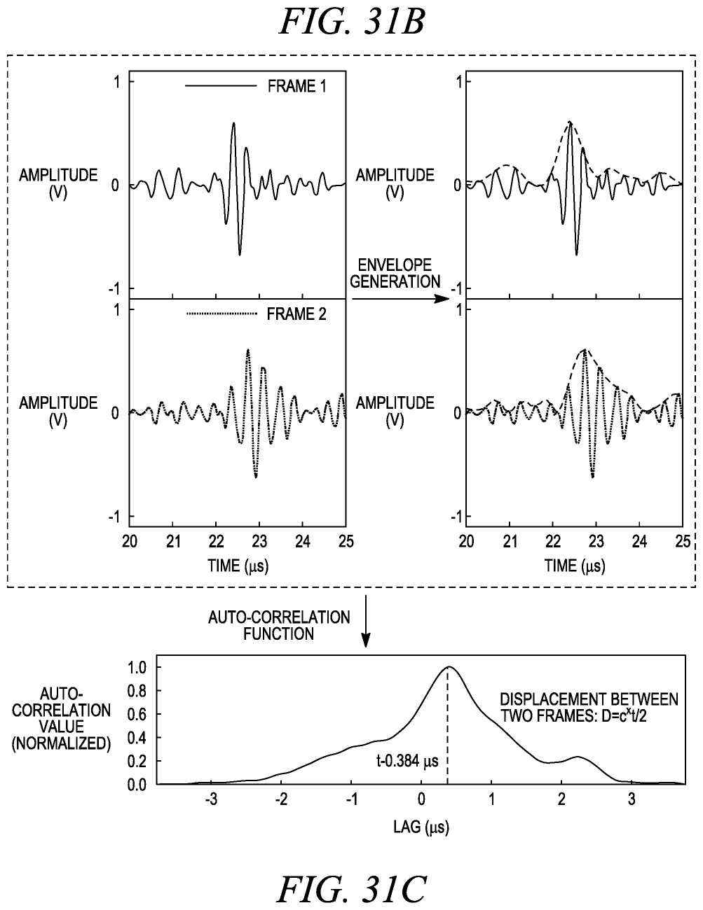

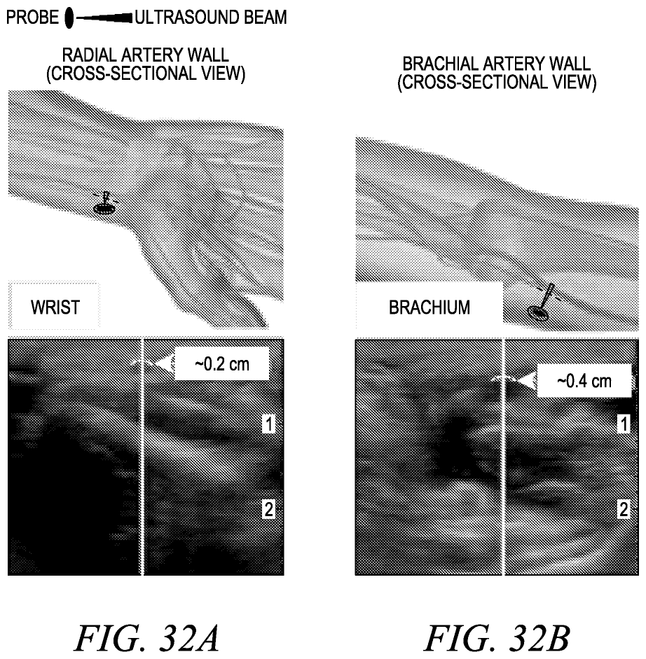

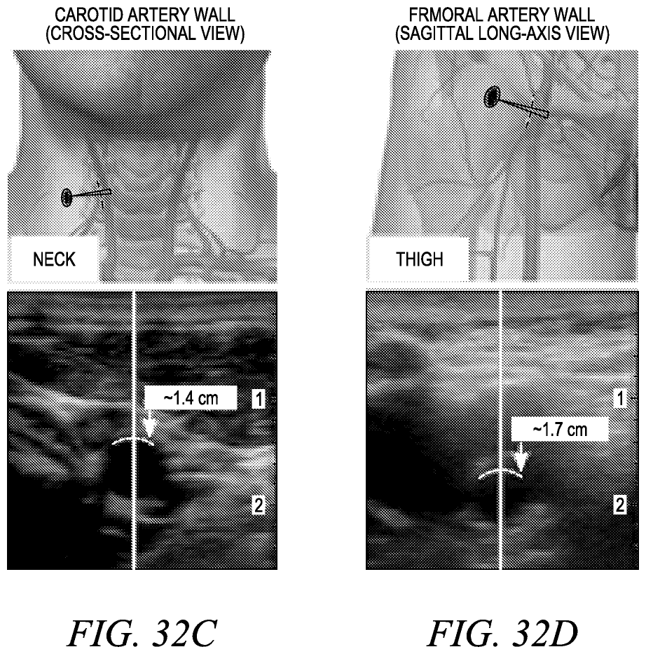

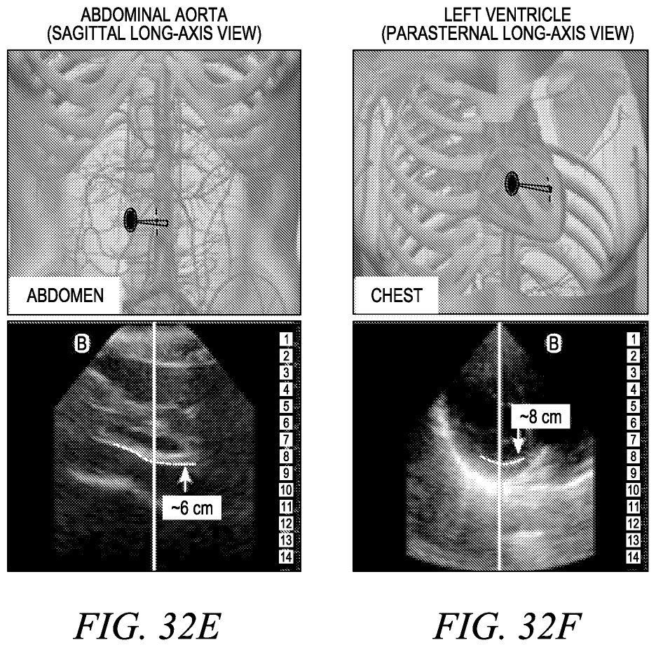

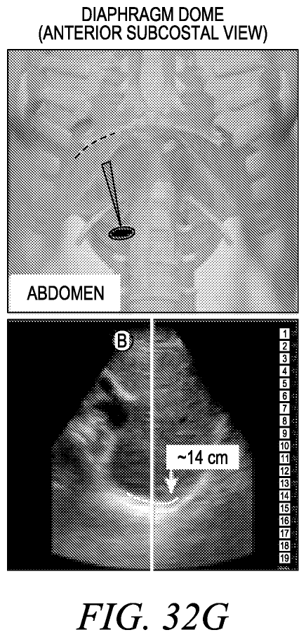

In clinical practice, A-mode and B-mode are commonly used for temporary measurements, while M-mode is for monitoring signals continuously. Additionally, M-mode is valuable for quantitatively characterizing tissue dynamics. Therefore, in this work, we focus the use of the USoP in M-mode. Natural physiological processes, such as circulation and respiration, can be manifested in the motion of tissue interfaces, such as myocardial contraction, arterial pulsation, and diaphragmatic excursion. The USoP can quantify these interfacial motions from multiple sensing windows in the human body (see FIG. 2a, FIGS. 31-32, the section below entitled Measurements of tissue interfacial motions, and Table 2).

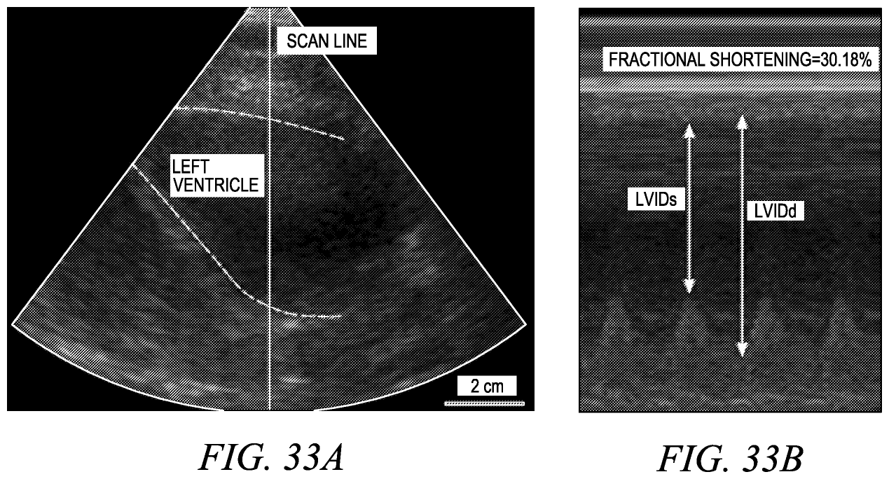

From myocardial contraction, the diameter change of the left ventricle during cardiac cycles can be recorded, and therefore fractional shortening can be derived as a measure of left ventricular function (FIG. 2b left). A comparison of measurements from the USoP and a commercial ultrasonic system shows a mean difference of ˜1% (FIG. 2b right, FIG. 33).

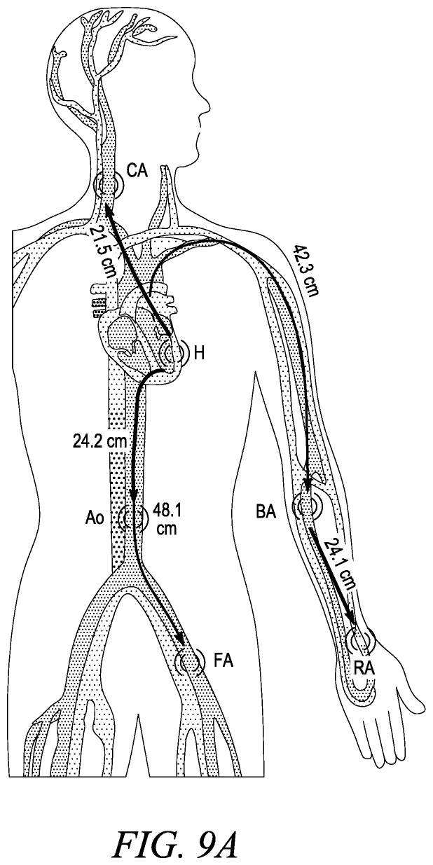

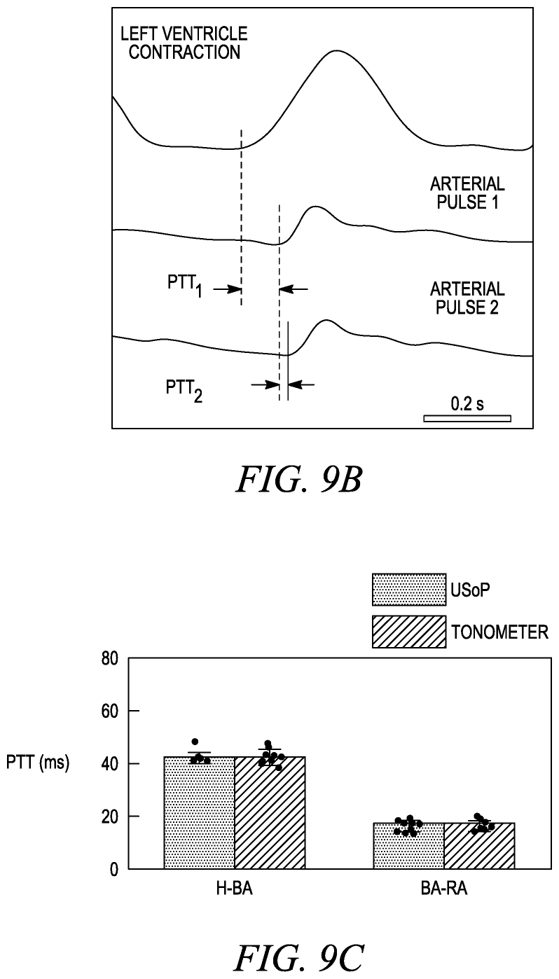

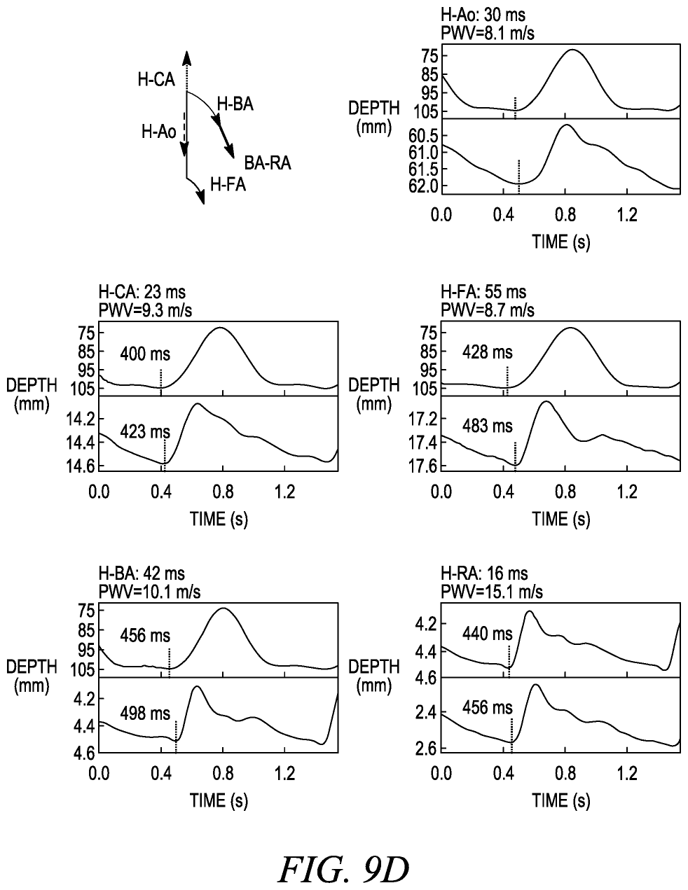

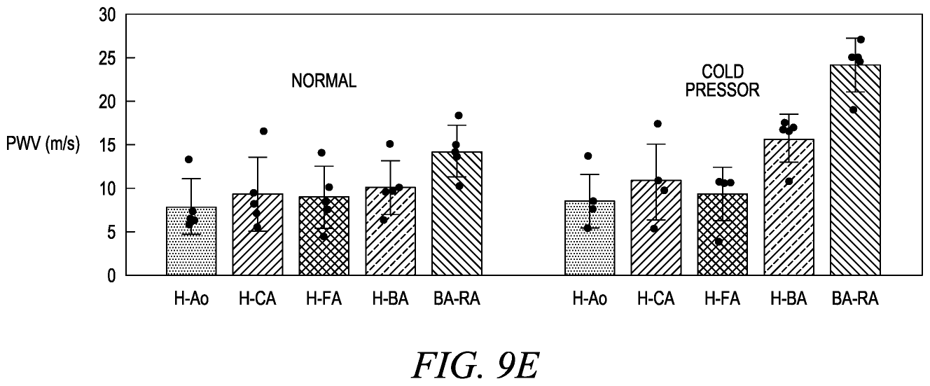

In arterial pulse waveforms, the pulse interval reflects the heart rate, and the pulse intensity can be correlated to blood pressure (see FIG. 2c and the section below entitled Measurement and calibration of arterial blood pressure). We validated the USoP results against a clinical-grade tonometer, the non-invasive gold standard for pulse waveform recording (Methods). Bland-Altman analysis was performed to compare the waveform-derived heart rate and blood pressure from both devices (FIG. 2d). The 95% limits of agreement included >95% of differences between the results from the tonometer and USoP, showing measurement consistency between these two devices. Additionally, the time difference between myocardial contraction and arterial pulsations can be used to quantify the pulse wave velocity, which correlates to the arterial stiffness of specific arterial segments (see FIG. 9 and the section below entitled Pulse wave velocity measurements). Comparing the results of the USoP with those of the tonometer suggests a mean pulse transit time difference of <0.5 ms, which results in <4% error in pulse wave velocity recording, further demonstrating the accuracy of the USoP (FIG. 9).

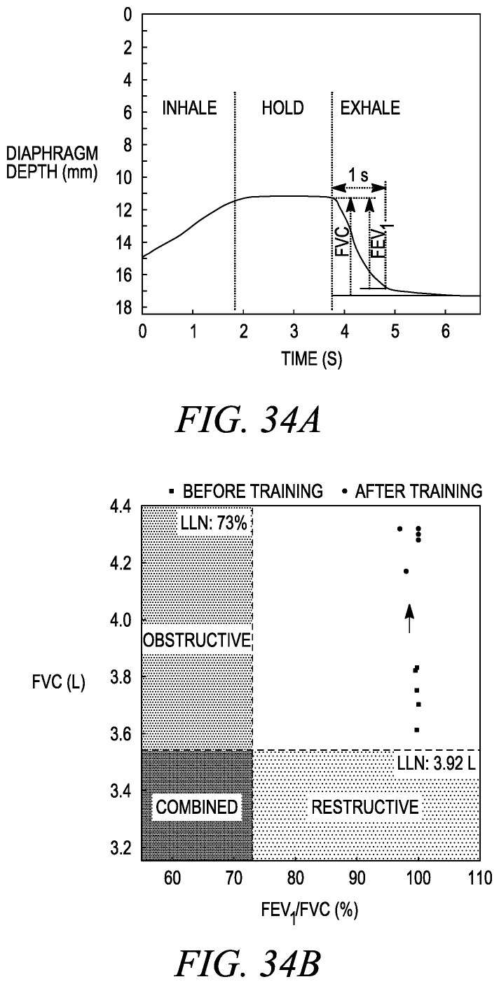

The USoP can also measure diaphragmatic excursion as a surrogate for changes in respiratory volume due to breathing. The diaphragm depth recorded by the USoP is compared with the respiratory volume recorded by a spirometer (FIG. 2e left and Methods). With a linear regression model, the correlation coefficients between the diaphragmatic depth and respiratory volume under normal and forced breathing conditions are 99.9% and 99.7%, respectively (FIG. 2e right). Furthermore, these derived volumes can be used to characterize the respiratory performance and identify airway obstruction or lung capacity restriction (see FIG. 34, the section below entitled Evaluation of respiratory function based on typical expiratory volumes and Table 3), which can potentially be used for screening respiratory issues such as chronic obstructive pulmonary disease and pulmonary fibrosis.

### Autonomous Data Acquisition and Analysis by Machine Learning

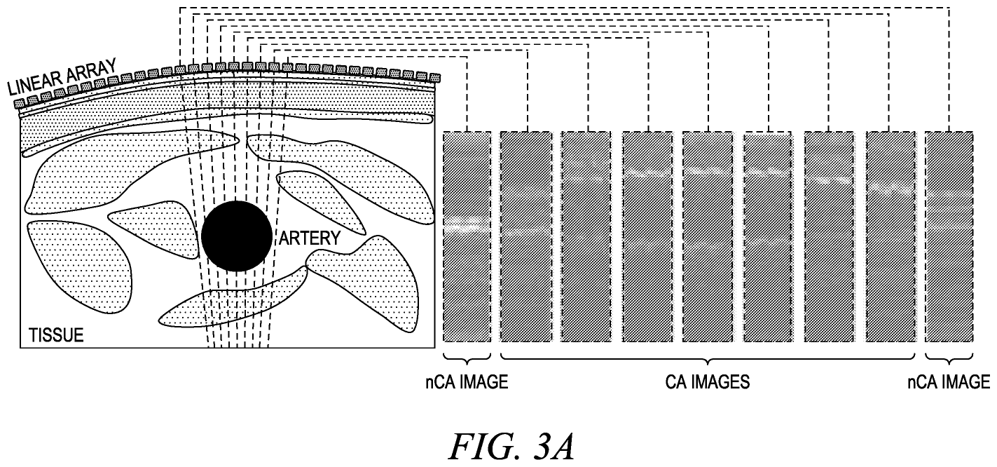

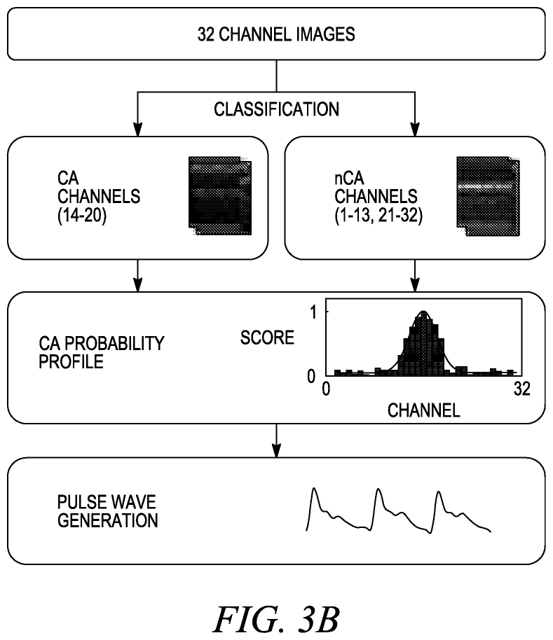

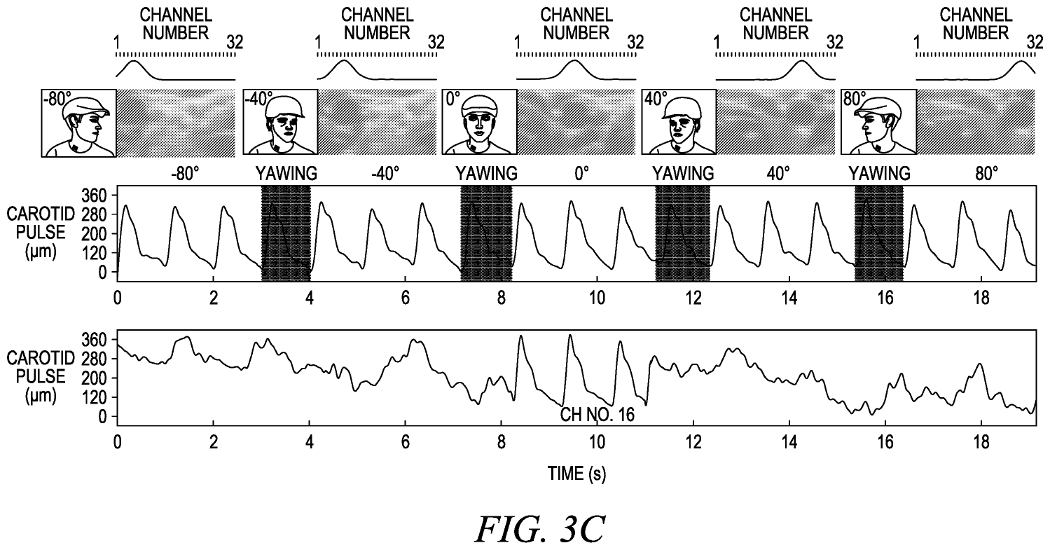

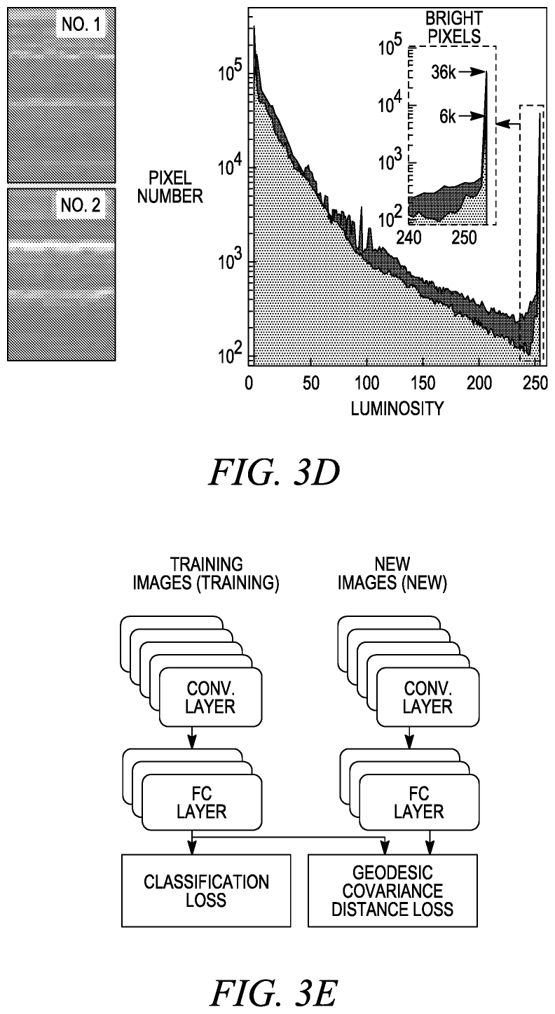

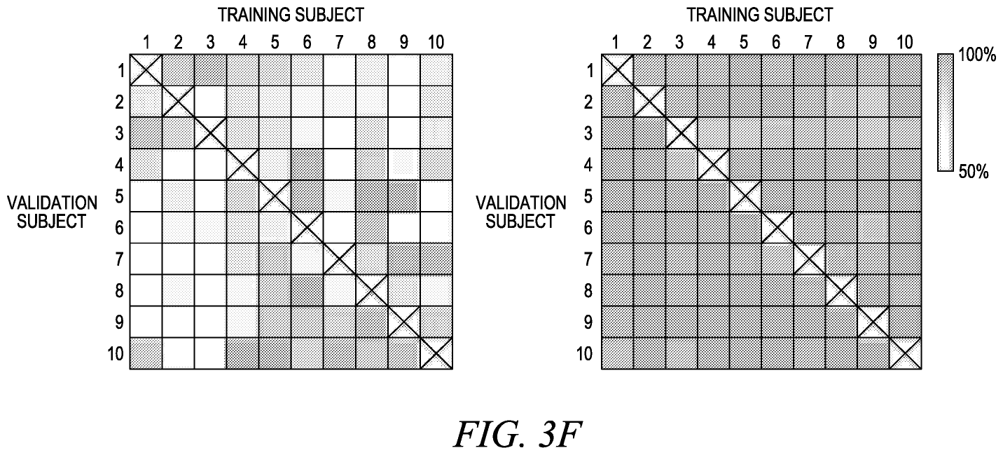

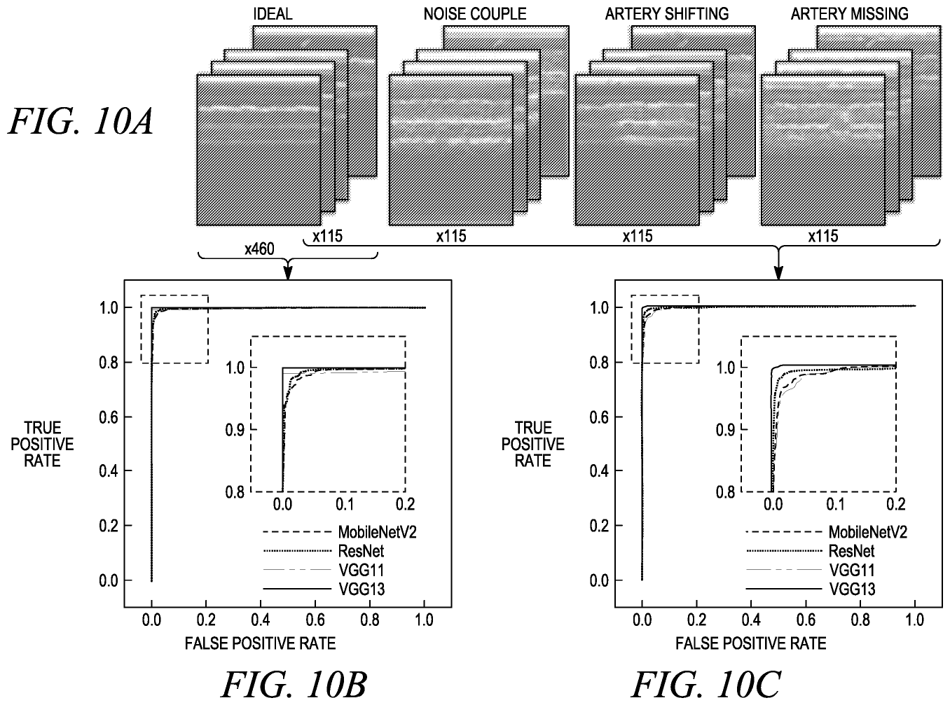

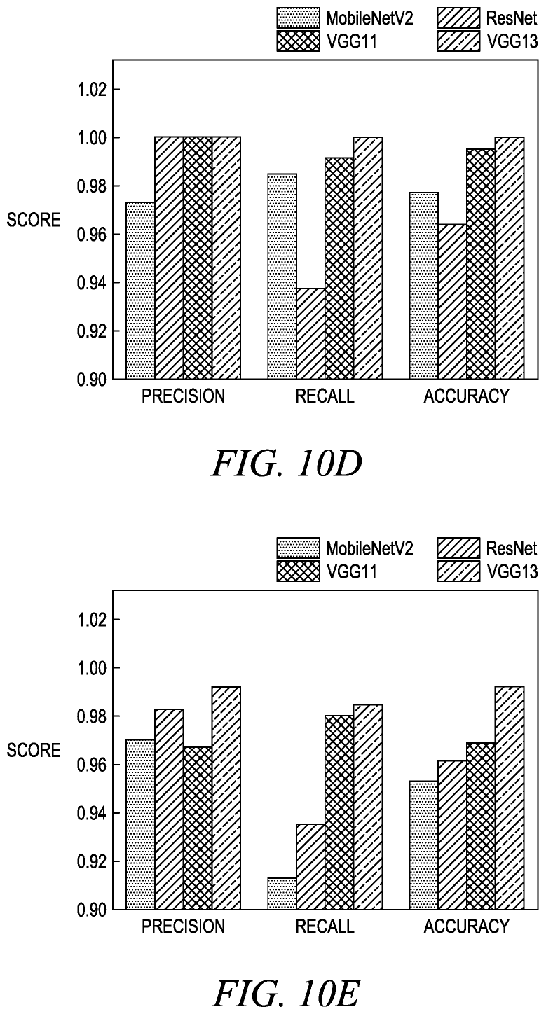

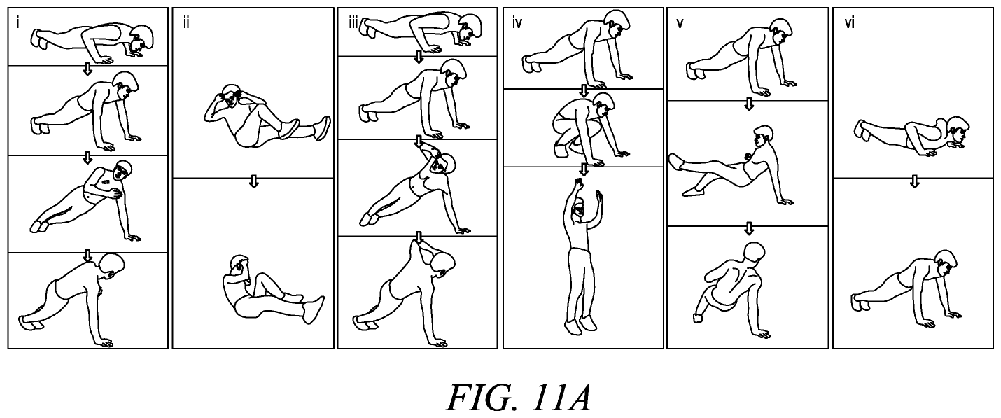

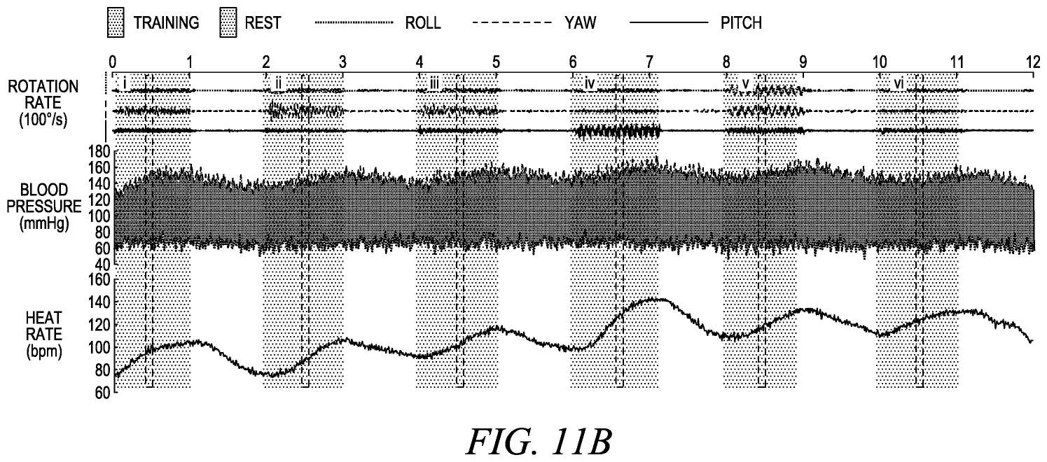

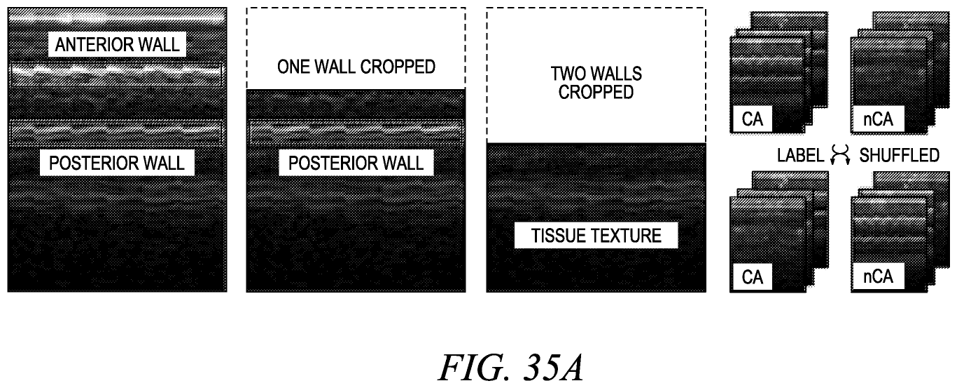

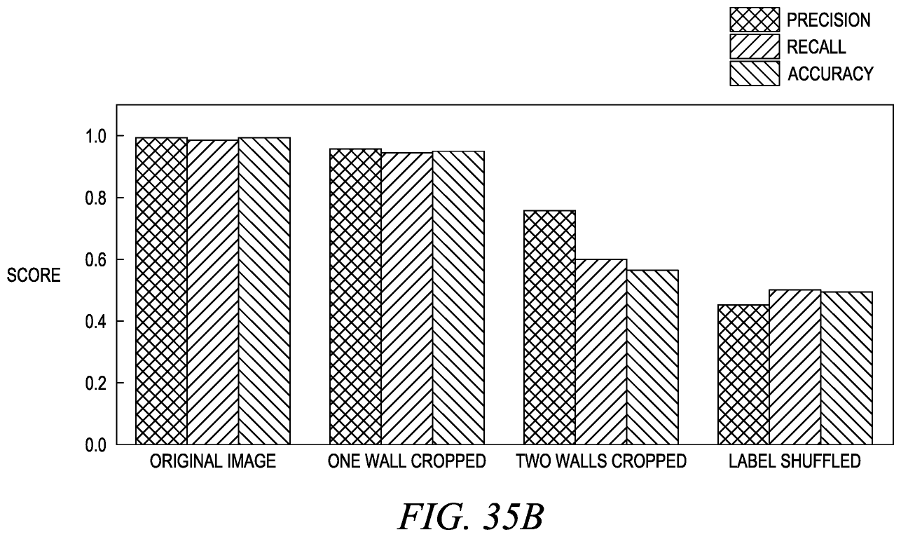

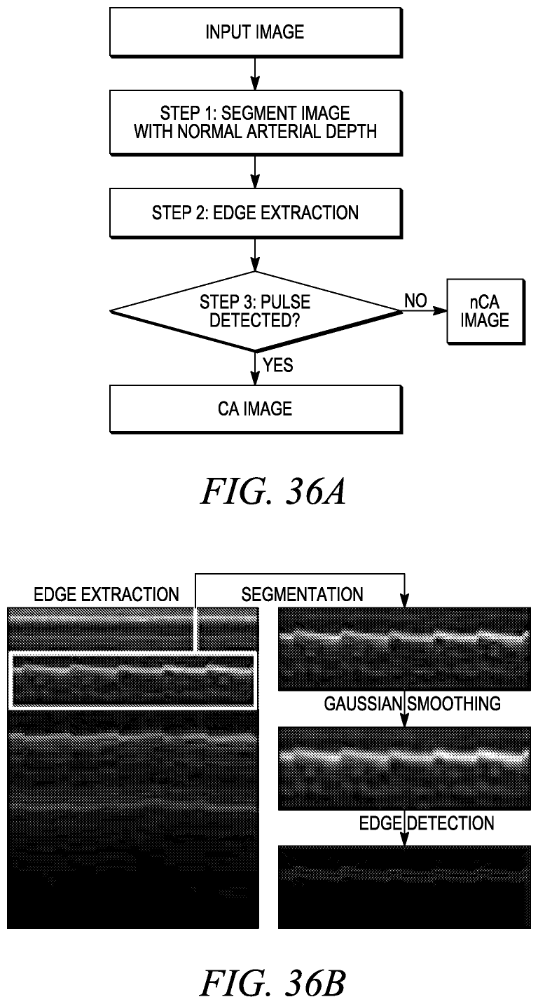

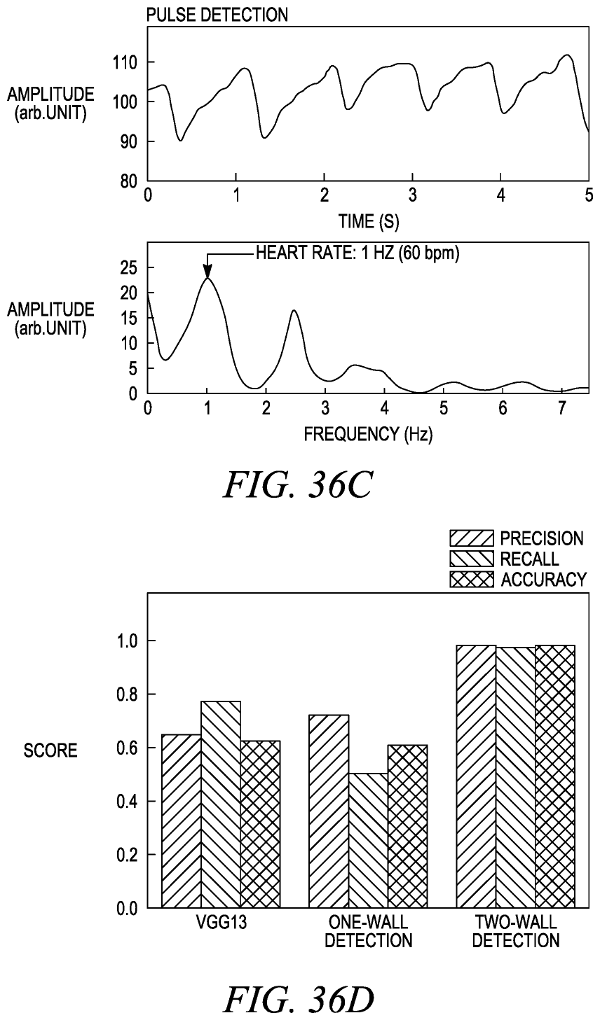

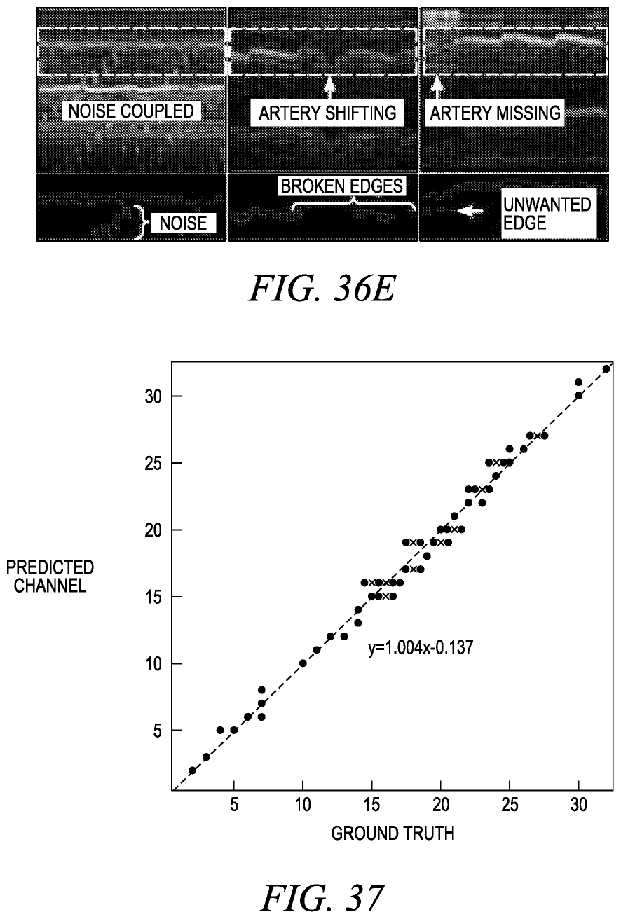

We use the USoP with a 4 MHz 32-channel linear array probe to autonomously and continuously track the position of the carotid artery and sense its pulsations. The linear array has an acoustic aperture of ˜25.4 mm, which is sufficiently wide to accommodate the misalignment between the probe and the carotid artery. Pulsation is visible in the M-mode images derived from the transducer channels directly above the carotid artery, while the M-mode images from the other adjacent channels show weaker or no pulsations (FIG. 3a). We train machine learning models to classify those M-mode images and identify whether salient pulsation patterns are present in the image (FIG. 35). Specifically, we use a VGG13 model because it outperforms other commonly used models for medical image classification in terms of precision, recall, and accuracy. This model can even robustly handle compromised ultrasound images and maintain the precision, recall, and accuracy higher than 98.4% (see FIG. 10 and the section below entitled Performance validation of deep learning models and comparison with logistic models), which more robust performance than conventional logistic models (FIG. 36 and the section below entitled Performance Validation of Deep Learning Models and Comparison with Logic Models). Based on the arterial wall patterns in the M-mode images, this model predicts probability scores for each of the 32 channels and, therefore, generates a probability profile of the position of the artery (see the section below entitled Probability profile generation from the prediction results). The channel with the highest probability is determined as the center of the artery (FIG. 37), and its channel data is used for generating the pulse waveforms (FIG. 3b).

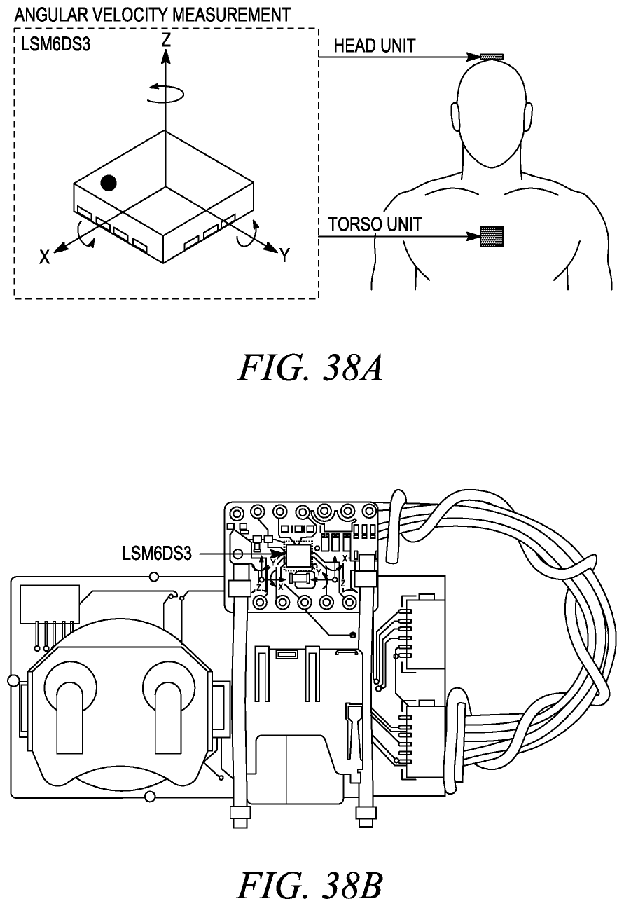

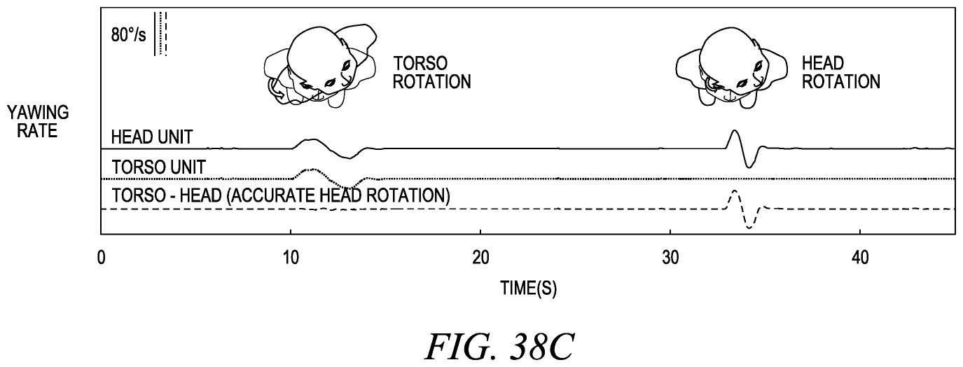

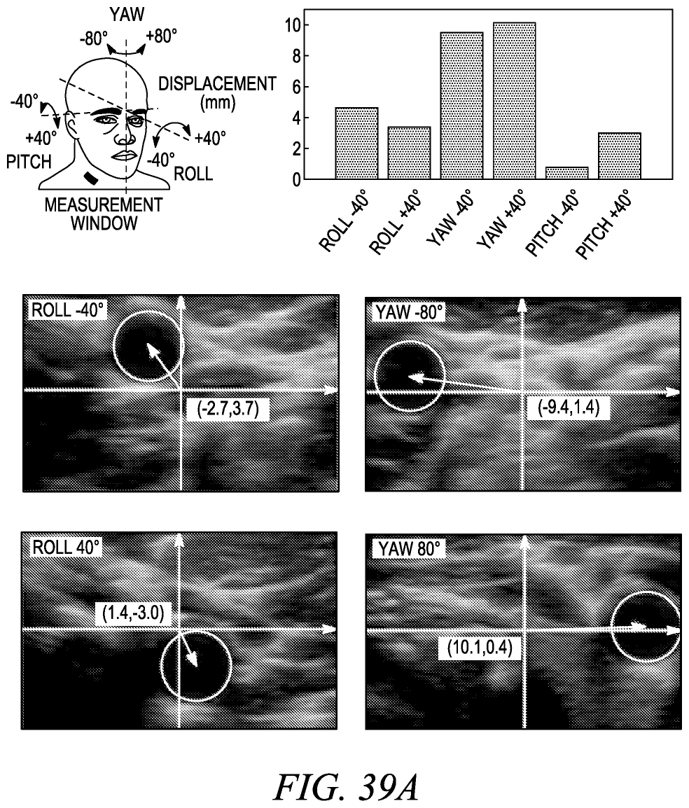

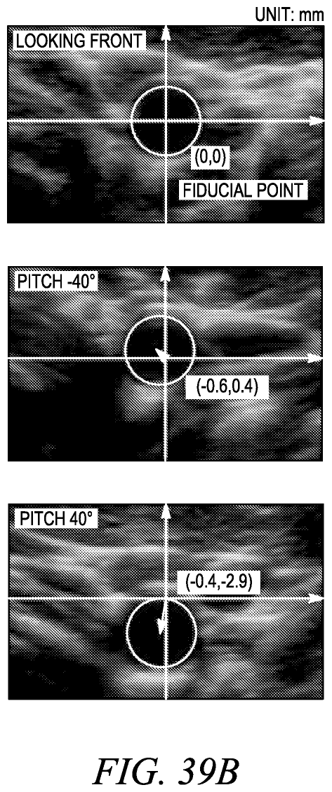

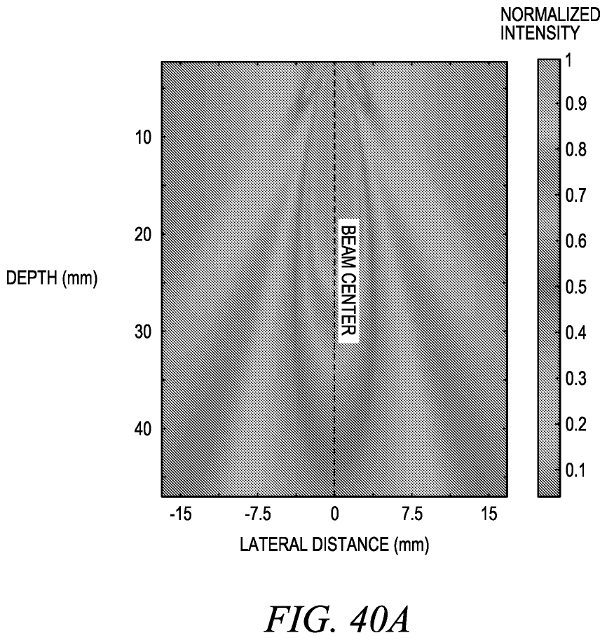

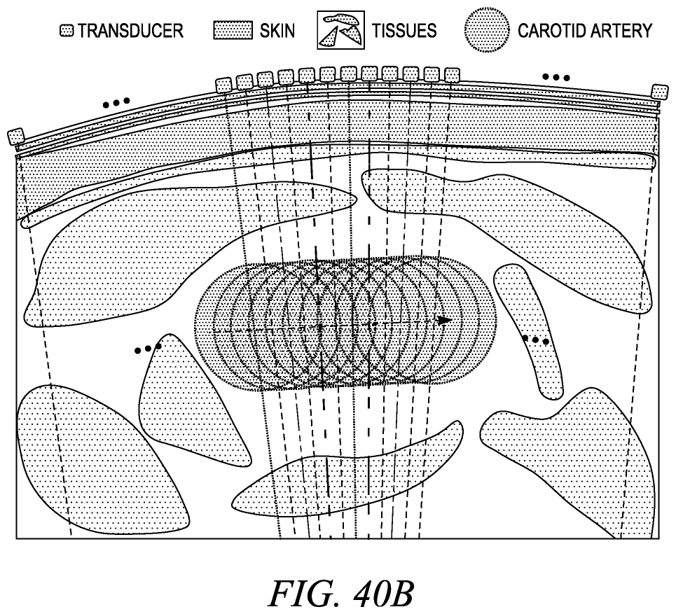

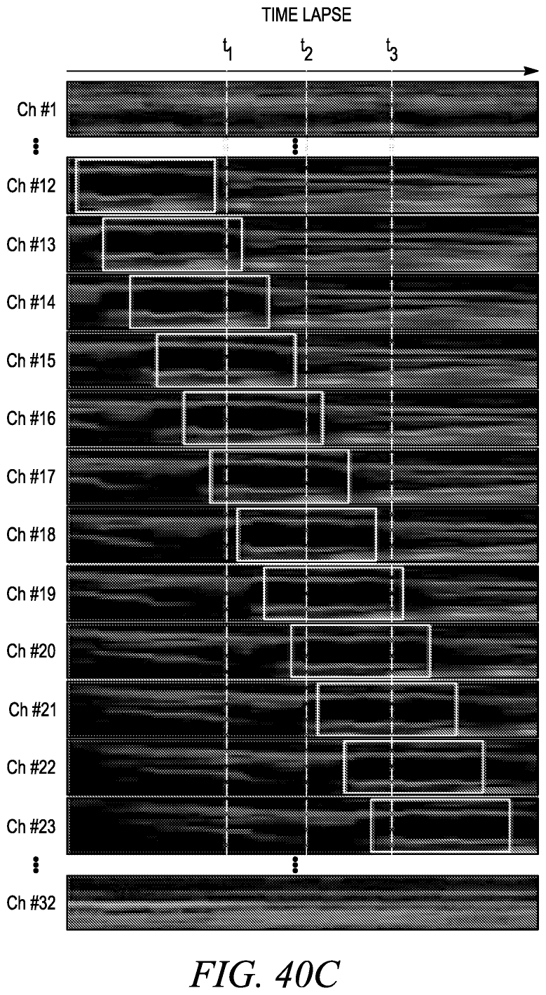

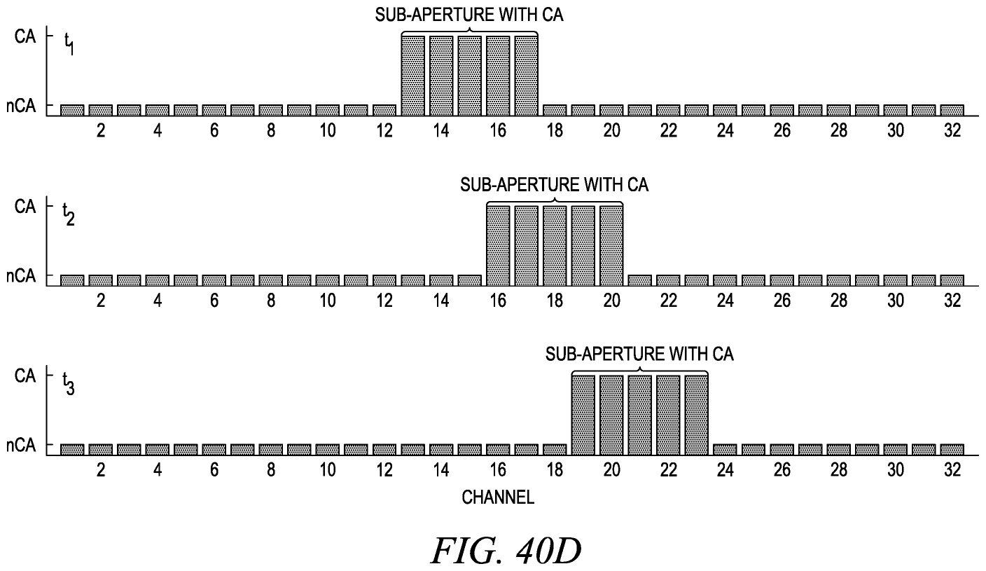

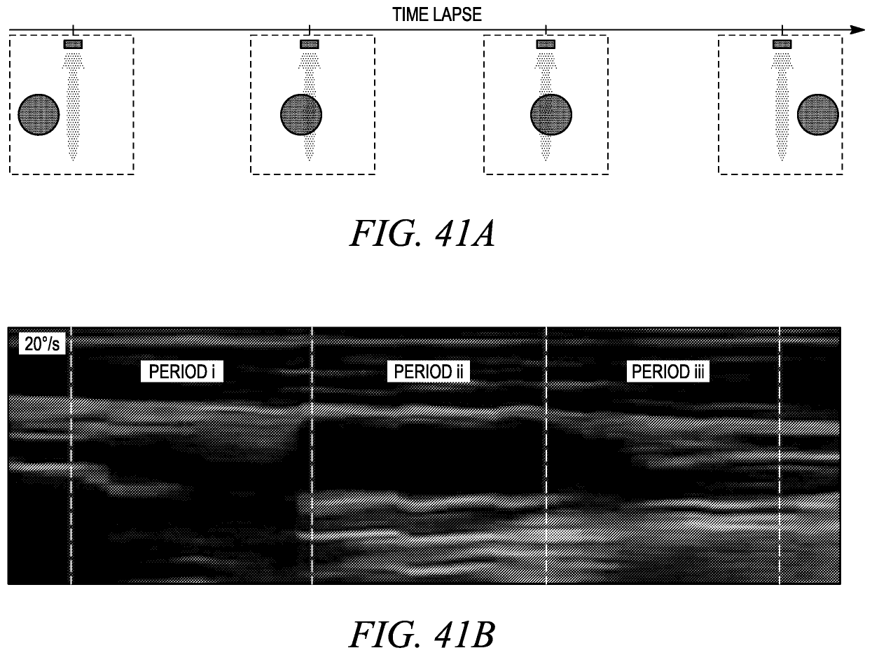

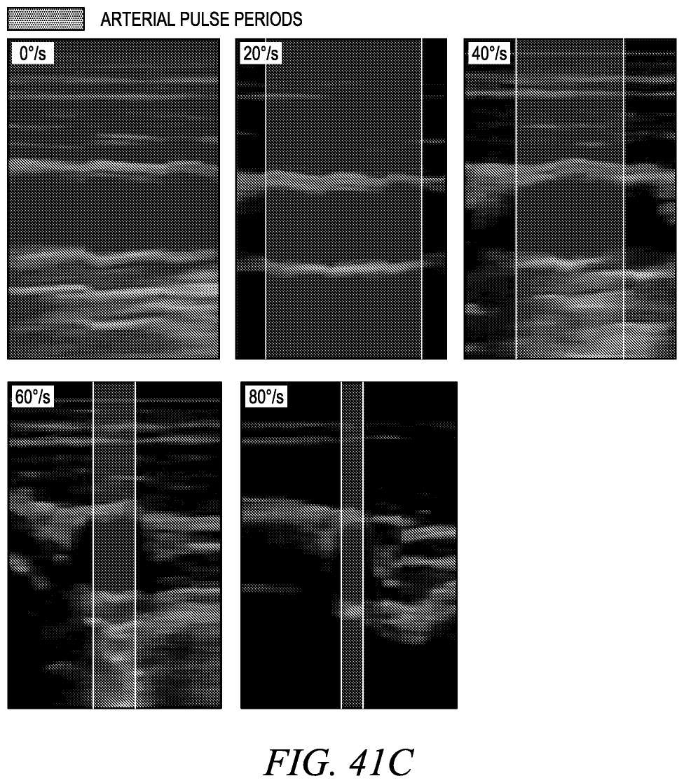

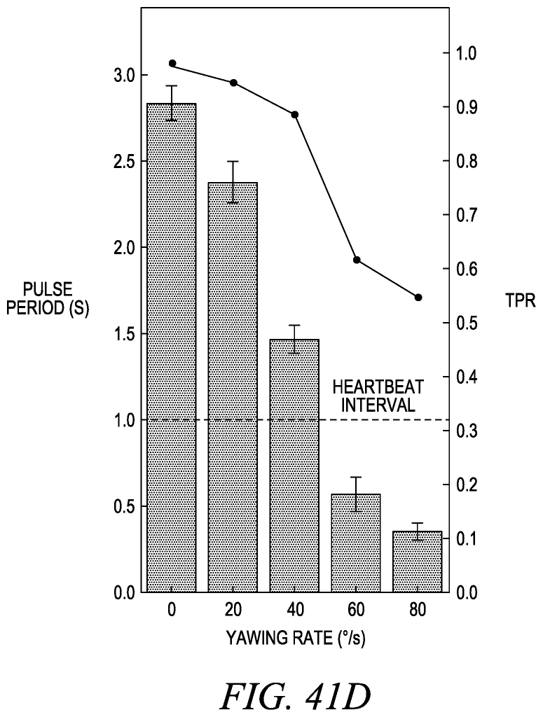

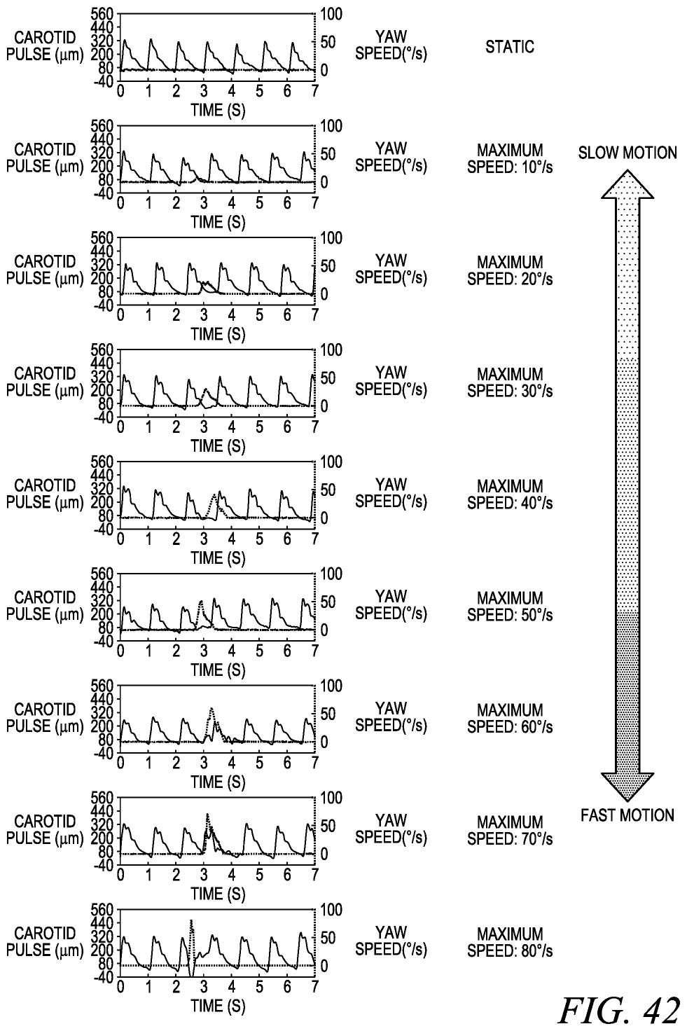

We record human head motion using inertia measurement units (FIG. 38 and Methods) and simultaneously image the carotid artery to quantify its displacement. The head can yaw at a larger angle than it can roll and pitch, and yawing generates the largest arterial displacement (>10 mm) (FIG. 39). The USoP generates M-mode images from all channels with head yawing. The VGG13 model identifies the M-mode images containing arterial pulsations, determines a moving sub-aperture to follow the carotid artery (FIG. 40), selects the optimal channel from the probability profile, and generates continuous pulse waveforms autonomously (FIG. 3c). In contrast, without the model, a fixed channel with head yawing generates inaccurate pulsation measurements or loses track of the pulsation waveform once the artery is outside its sensing aperture (FIG. 3c). The model prediction remains reliable at a head yawing rate <60°/s (see FIG. 41 and the section below entitled The limit of motion tolerance and pulse waveform continuity). At yawing rates beyond this limit, the pulse waveform becomes distorted but is quickly restored when motion stops (FIG. 42).

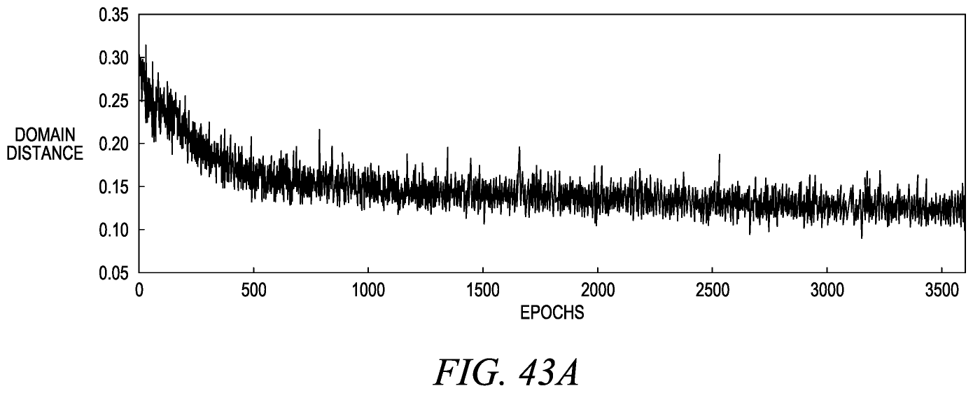

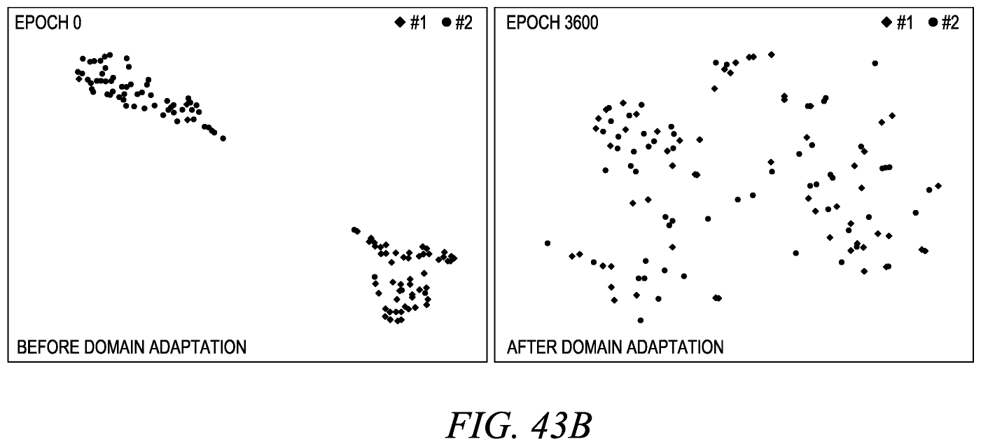

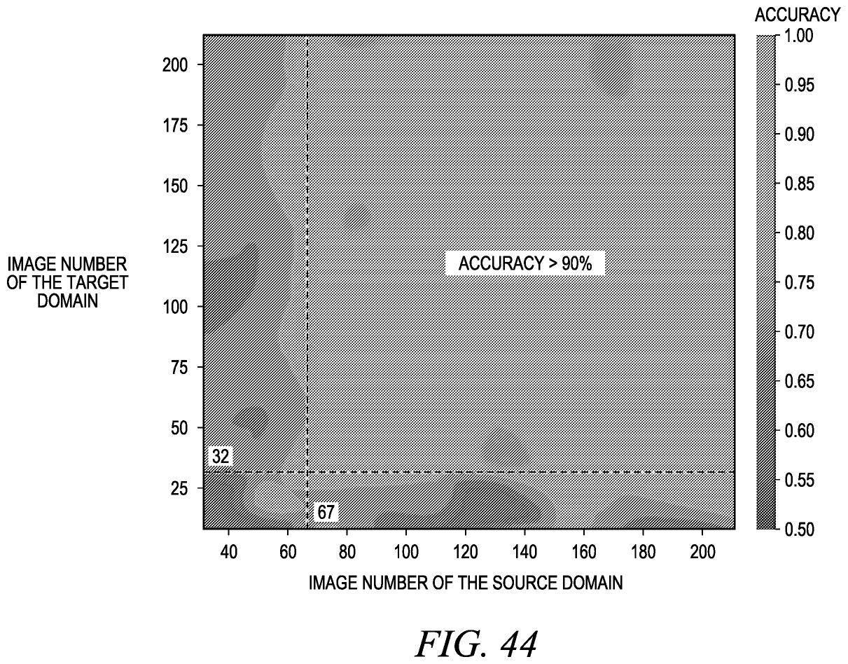

Machine learning algorithms may encounter generalization problems when tested on images outside the training pool. For example, images from a new subject may have distinct brightness, contrast, and arterial wall patterns, which would result in different luminosity distributions (FIG. 3d). We enhanced the generalization of the VGG13 model by using domain adaptation with a minimal entropy correlation alignment model (see FIG. 3e and the section below entitled Training principles of a minimal entropy correlation alignment (MECA) model) to transfer the machine learning network to new image datasets without additional labeling. The use of domain adaptation allows the model to generalize to different subjects. A t-distributed stochastic neighbor embedding visualization of the subject distributions shows that images from different subjects are unified after domain adaptation is applied (FIG. 43 and Methods). Model generalizability is demonstrated through cross-validation among 10 subjects (Table 4). We train the classification model on each subject and then validate it on the nine other subjects. Without domain adaptation, the model only has an average accuracy of 63.23% on new subjects (FIG. 3f left). After domain adaptation, this accuracy increases to 96.13% (FIG. 3f right). We also investigate the minimum data required to be collected from a new subject for successful domain adaptation. The results showed that only 32 unlabeled images from a new subject suffice to achieve >90% classification accuracy (see FIG. 44 and the section below entitled Dataset size required for domain adaptation).

### Continuous Monitoring During Exercise

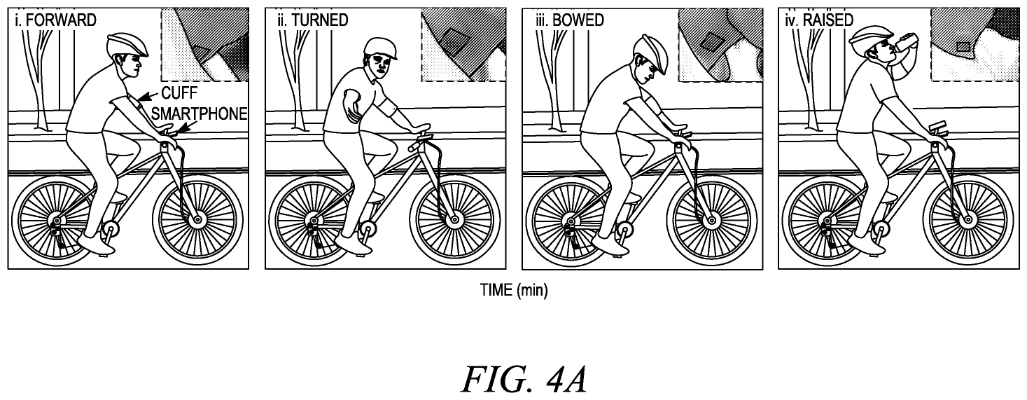

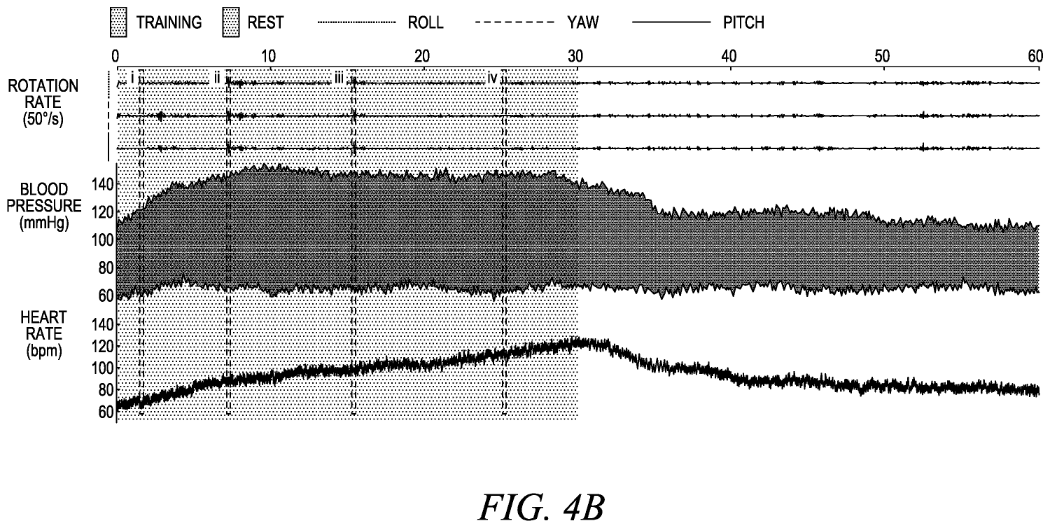

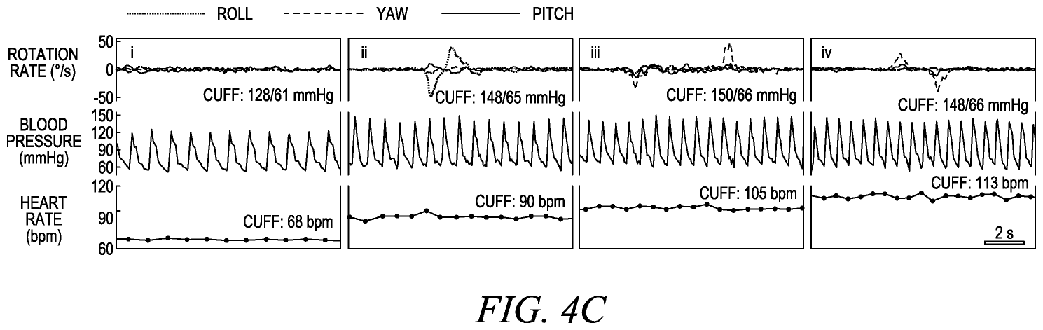

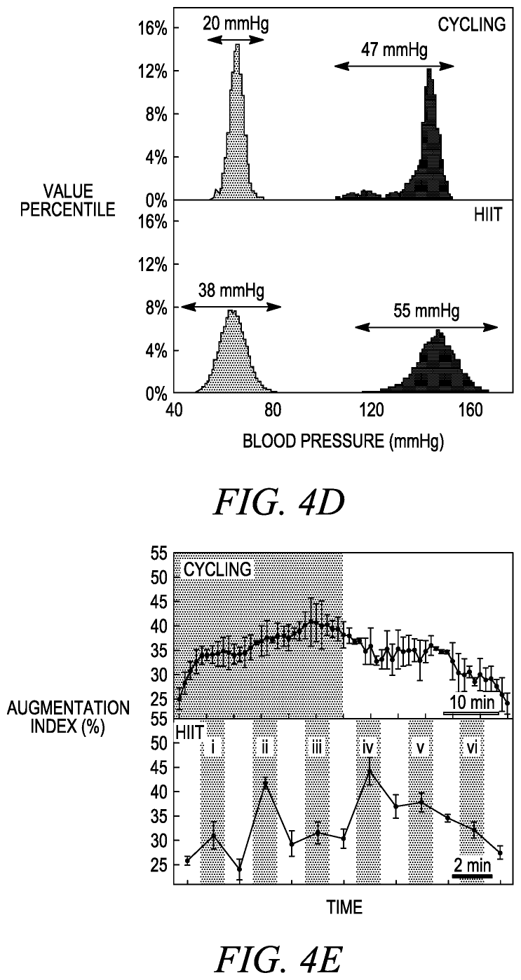

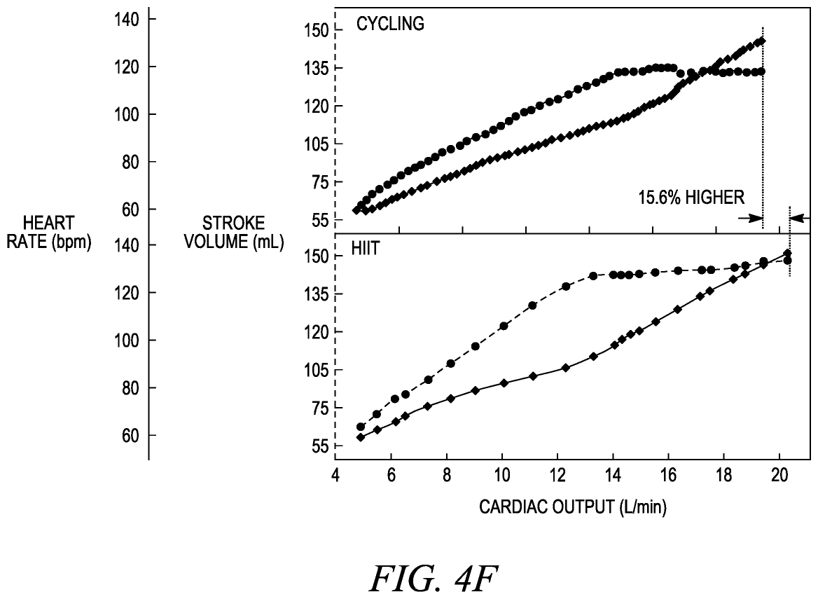

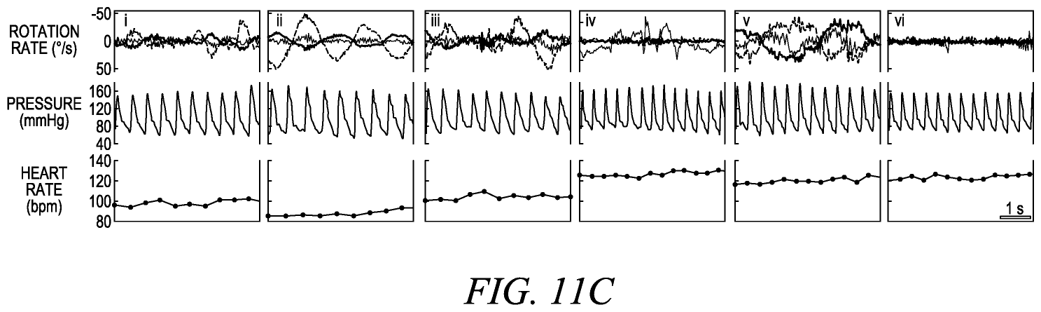

The USoP can continuously track multiple deep tissue signals during human motion. To test its performance, we use it on a participant during aerobic exercise, when the participant performed 30 min continuous cycling followed by 30 min rest. We record the carotid blood pressure waveform while the participant moves freely (FIG. 4a). Similar measurements are made during anaerobic exercise, when the participant performed high-intensity interval training (HIIT) comprised of six one-minute training sessions, separated by six one-minute periods of resting (FIG. 11).

Upon the onset of exercising, the substantial increase in the blood pressure and heart rate suggests a boost in circulating blood, also known as the stressed volume (FIG. 4b-4c). During both cycling and HIIT, the systolic pressure increases more than the diastolic pressure, regulated by increased cardiac output and decreased vascular resistance (see the section below entitled Systolic and diastolic blood pressure changes during exercise). The heart rate increases monotonically during both types of exercise and decreases during resting, as anticipated. As cycling progresses, the blood pressure gradually stabilizes at a relatively elevated level, resulting in narrow distributions of both systolic and diastolic pressures in the histogram (FIG. 4d upper). These results imply that the systemic vascular resistance decreases to a physiologically determined steady state to support prolonged muscle activity. This is in stark contrast to HIIT, during which blood pressure fluctuates, resulting in wider distributions of both diastolic and systolic blood pressures (FIG. 4d lower). In both cycling and HIIT, resting allows blood pressure to gradually decrease toward the baseline.

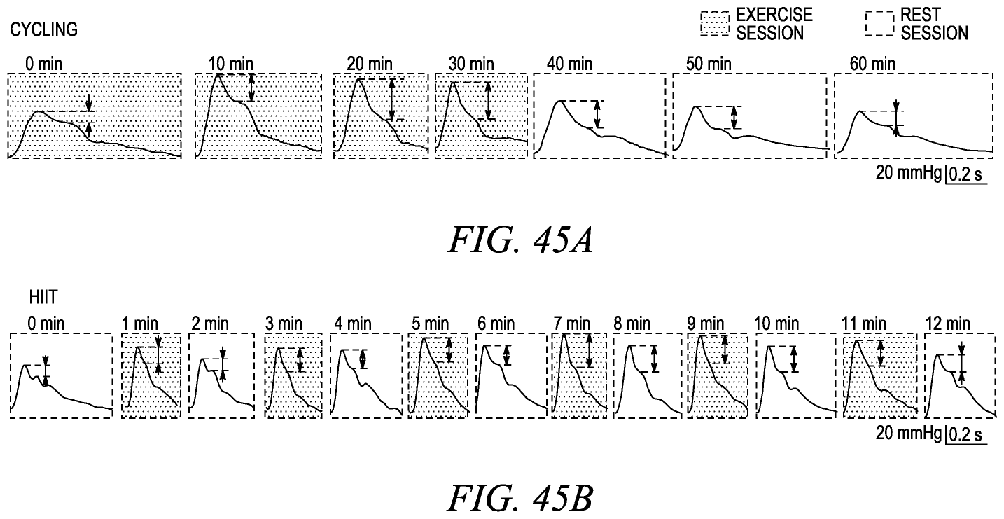

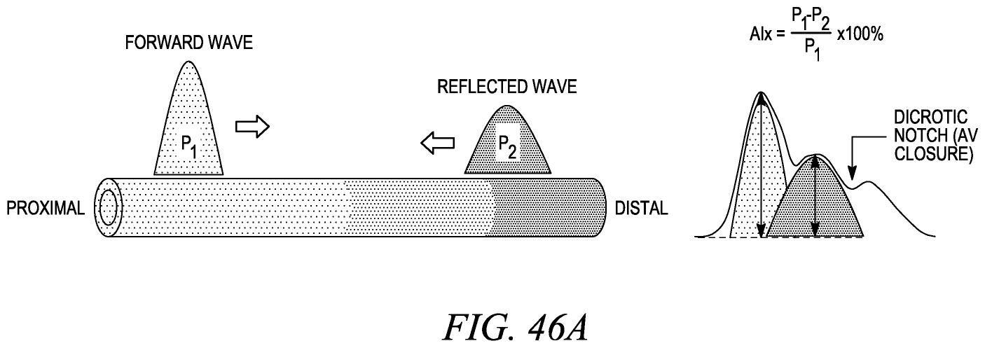

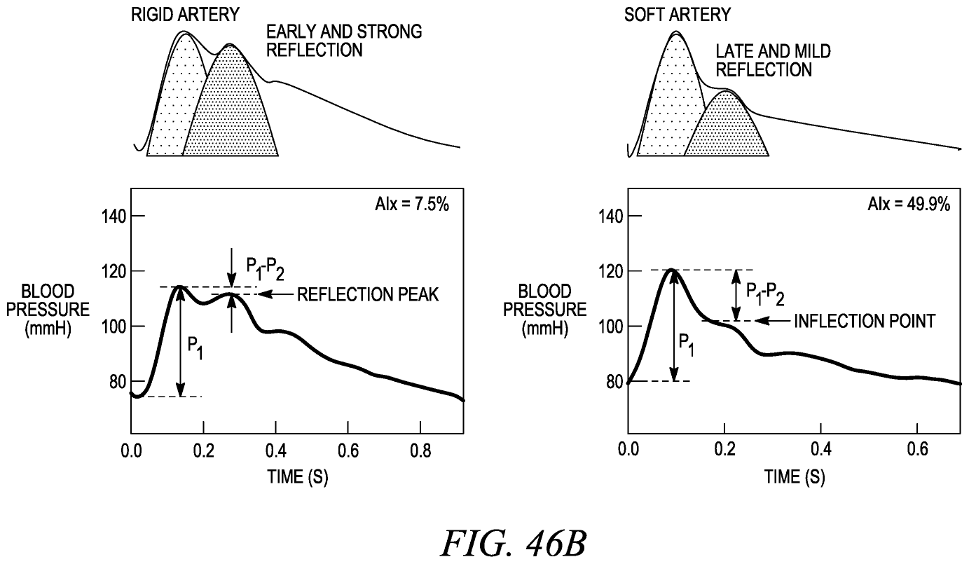

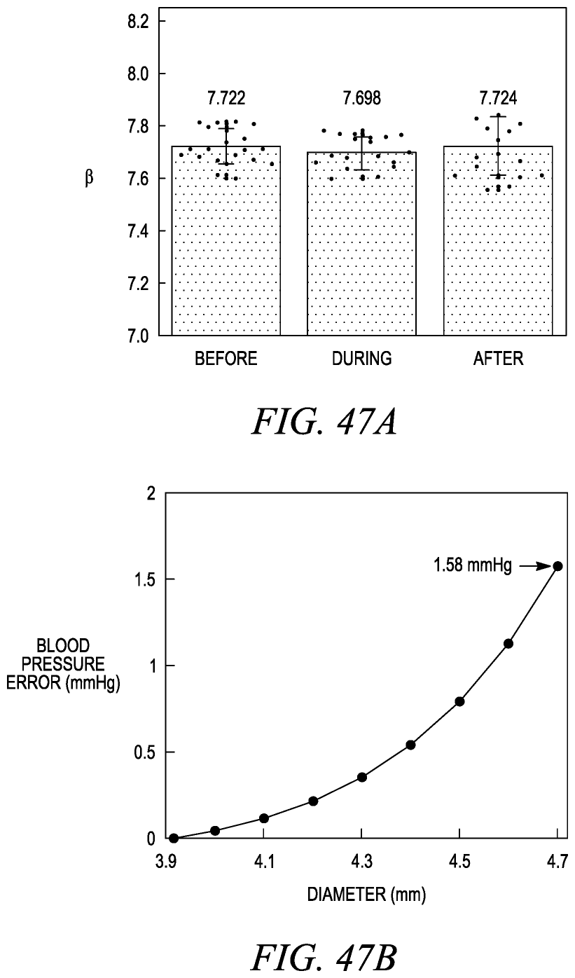

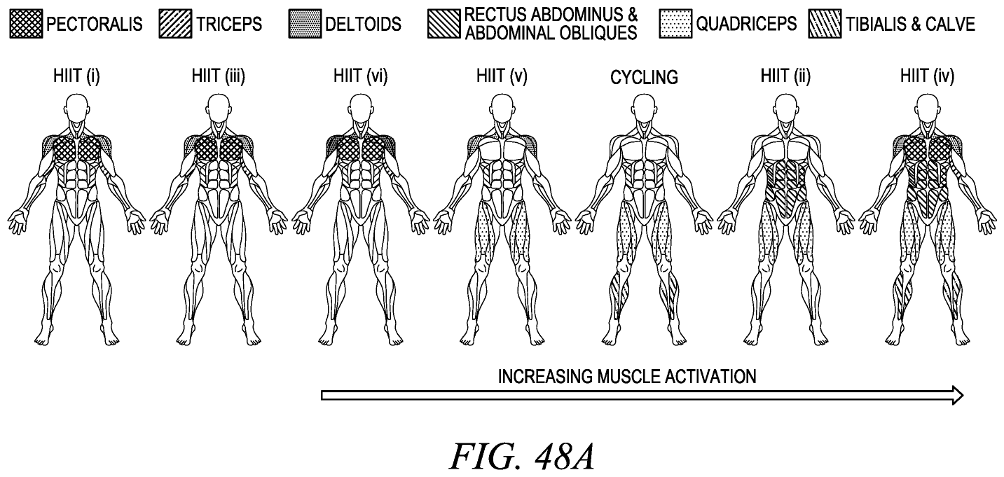

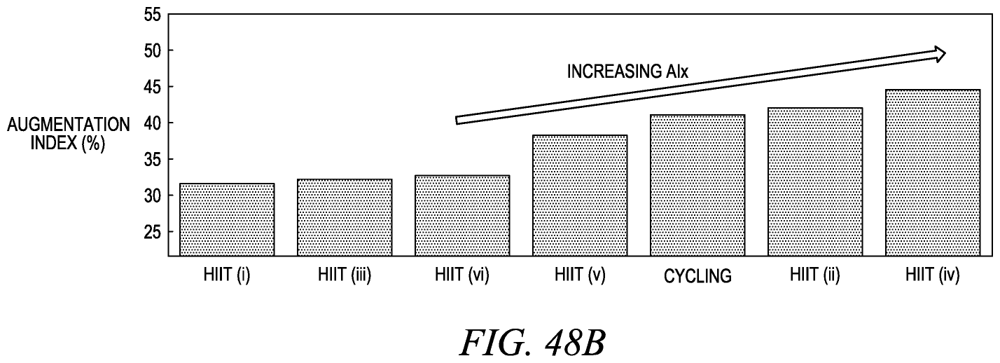

We derive the vascular responses to exercise by calculating the augmentation index (see FIGS. 45-46 and the section below entitled Quantifying the vascular response to exercise). In both cycling and HIIT, the augmentation index increases with exercise and recovers with resting; when the exercise is sufficiently long, as in the case of cycling, the augmentation index stabilizes (FIG. 4e). The increase in the augmentation index during exercise may have two causes: vessel stiffening and vasodilation. We measure the change in the arterial stiffness index before, during, and after exercise (FIG. 47 and Methods). The results suggest a negligible change (<0.34%) in the stiffness index. Additionally, such a negligible change in the stiffness index leads to a central blood pressure error <1.58 mmHg after calibration, which proves the reliability of the blood pressure recordings during exercise (see FIG. 47 and the section below entitled Changes in arterial stiffness index and errors in blood pressure calibration during exercise). Therefore, the increase in the augmentation index is primarily driven by vasodilation rather than changes in arterial stiffness. The vasodilation takes place mainly in the skeletal muscle involved in the exercise to support an elevated demand for oxygen and thus blood flow; activating larger muscle groups results in greater vasodilation and increased blood flow, and thus a higher augmentation index (FIG. 48).

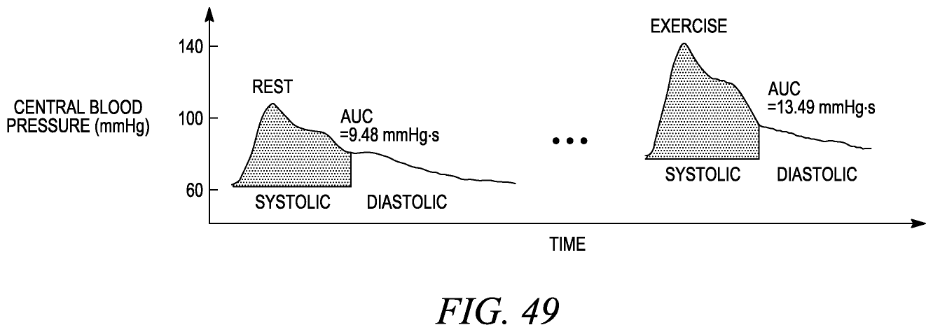

We estimate the stroke volume from the pressure waveforms using a pulse contour method (see FIG. 49 and the section below entitled Stroke volume estimation using the pulse contour method). The cardiac output is then calculated as the product of stroke volume and heart rate. Similar patterns in the stroke volume and heart rate are observed in both cycling and HIIT (FIG. 4f). The measured cardiac output increases as the exercise intensifies, and the heart rate increases together with the cardiac output. Initially, the stroke volume increases before plateauing as end-systolic volume approaches the mechanical limits of the heart and the increase of end-diastolic volume begins to be limited by the shorter filling times at higher heart rates. In the high cardiac output region (e.g., >15 L/min), the stroke volume plateaus, and the increase in cardiac output is mainly attributed to the increase in heart rate. Compared to cycling, HIIT produces a greater increase in stroke volume and a higher maximum cardiac output, indicating that HIIT may be a more effective training modality for enhancing cardiac functions.

### DISCUSSION

While most existing wearable devices capture signals on or near the skin surface, such signals are often manifestations of physiological processes in deep tissues. Therefore, in many clinical applications, it is critical to monitor deep tissue signals directly. More importantly, deep tissue physiology is constantly changing. To identify potential risk factors for a disease, capture its early onset, or evaluate its progression, obtaining longitudinal data over the course of days, weeks, or even months is key. This calls for a tool that enables long-term deep tissue surveillance, processes the data stream in real time, and remains accurate during human motion.

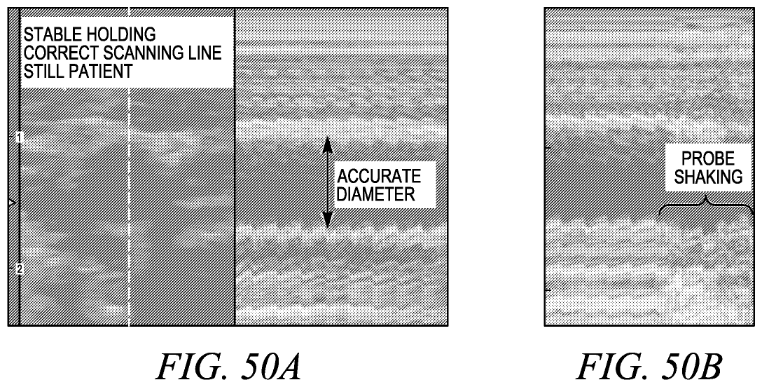

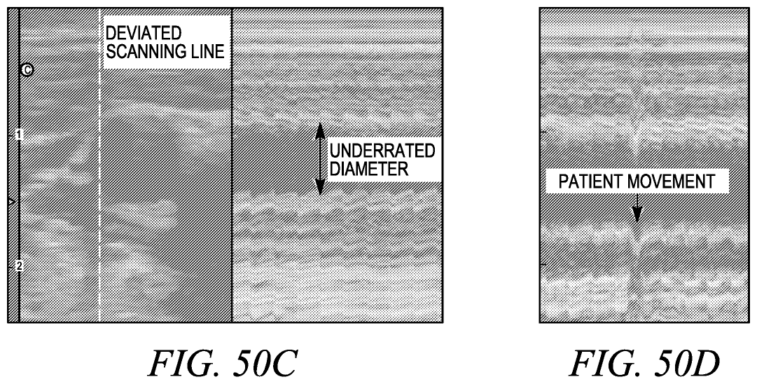

Medical ultrasound is one of the most widely used methods for deep tissue sensing, but due to the complex equipment and the requirement for an operator, traditional ultrasound exams offer point-in-time measurements only. In fact, one of the barriers that prevents traditional ultrasound from long-term use is its operator dependency. Even with standardized exam procedures, results reported using conventional ultrasonography strongly depend on operator skill. When mishandled, manual ultrasonography may generate compromised or even erroneous results (see FIG. 50 and the section below entitled Errors in conventional ultrasonography).

Recent advances in wearable ultrasonography have shown the promise of capturing deep tissue signals over the long term. Soft wearable ultrasonic probes, as well as rigid ultrasound chips integrated with soft adhesives, have demonstrated hands-free ultrasound signal acquisition. However, removing the requirement to handhold the probe is only the initial step toward continuous operation, and three further technical barriers remain. First, these probes have to be wired to a central processing station, which largely limits the wearing subject's mobility. Second, existing wearable ultrasound devices face challenges with measurement continuity and reliability in moving subjects, because the device on the skin shifts in position relative to the target tissue. Third, wearables generate new challenges for manual data processing because any clinicians will be overwhelmed by the continuous data stream.

The fully integrated USoP addresses these three barriers and makes continuous surveillance of deep tissue signals possible. First, the USoP eliminates wire connections by connecting the device and the backend processing system wirelessly, which allows for large-range subject mobility. Second, the USoP uses machine learning-based algorithms to automate the data acquisition and channel selection in real time. To our knowledge, no previously reported wearable device can autonomously track a moving target. Third, deep learning-enabled data post-processing relieves the human burden and enables potential scale-up. Together, these innovations open up many new possibilities. For example, patients can be monitored as they conduct their natural daily activities, which can provide rich information that is more clinically relevant. Responses to high-risk activities such as during an intense workout can be captured for more rigorous diagnostics. Continuous monitoring over days or weeks of the dynamic changes of the cardiovascular system in response to stressors can benefit a broad range of populations, from athletes who need training optimization, to cardiac rehabilitation patients who require safety measures, and to general high-risk populations for cardiovascular risk stratification and prediction (see the section below entitled Clinical benefits of continuous monitoring during exercise).

A number of issues arise with the soft ultrasonic probes described herein. For instance, the soft ultrasonic probes face challenges of unknown transducer locations when conformed to dynamic and curvilinear skin surfaces. A-mode and M-mode using single transducers without beamforming are not affected, but unknown transducer locations cause phase aberration and compromised beamforming for B-mode imaging. Potential solutions include applying additional shape sensors to map the transducer locations in real-time, or developing iterative contrast optimization algorithms to compensate the phase distortion of a deformed array. Another issue concerns the long-term wearability of the USoP. Integrating highly integrated chips with multilayered soft circuitry could further enhance the mechanical compliance of the system. Combining wearable power harvesting devices could extend the battery life of the USoP. Replacing silicone adhesives with more durable and permeable adhesives could help enhance skin integration under skin deformation and perspiration. Yet another issue concerns the applicability of USoP to other tissue targets, particularly in high-risk populations where continuous monitoring is critical (see the section below entitled Clinical need for continuous tissue monitoring in high-risk populations). Yet another issue concerns the cloud computing resources necessary for machine learning processing, which can limit their accessibility in remote and undeveloped regions. On-board data analytics based on power-performance balance optimization and artificial intelligence-on-a-chip technology may be employed to address this issue. Finally, through strategically tuning the ultrasound controlling parameters such as activation frequency and pulse profile, this technology could enable more intriguing wearable diagnostic and therapeutic applications, including anatomic imaging, functional imaging, and ultrasound stimulation.

### Ultrasonic Probe Fabrication and Layout Designs

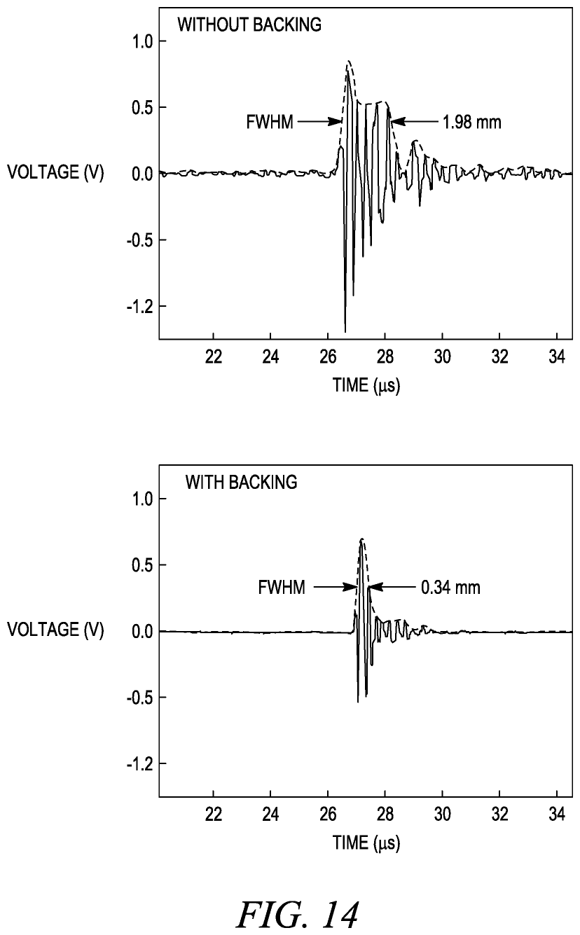

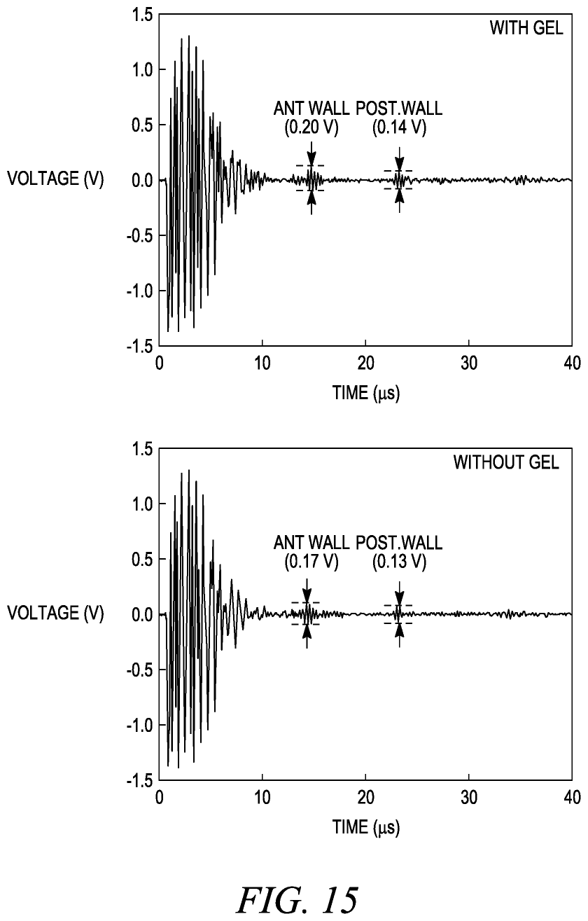

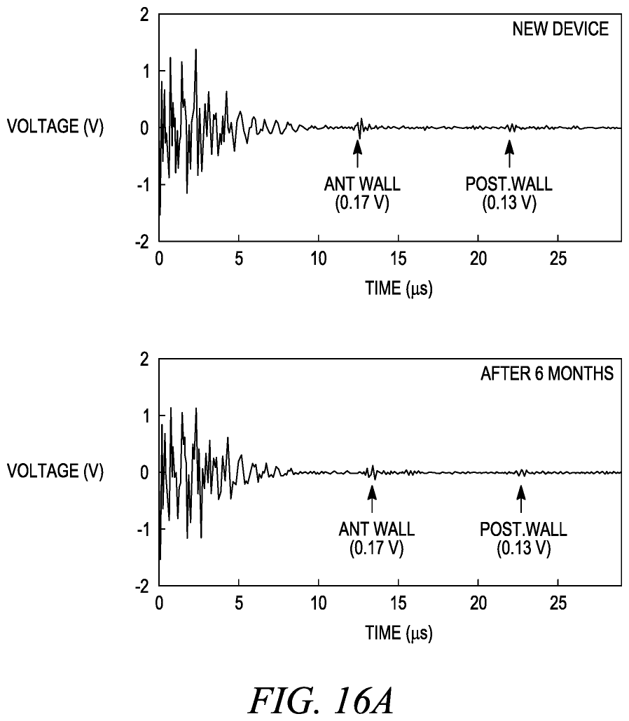

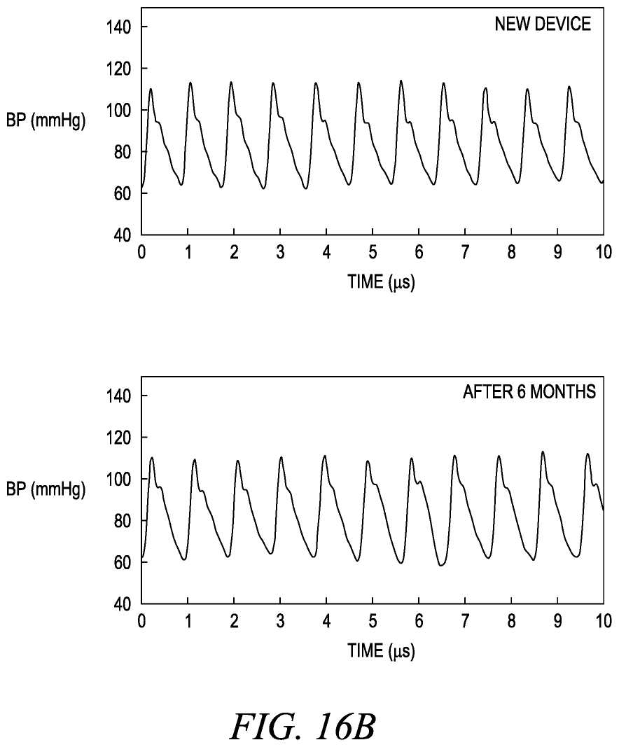

In some embodiments, the ultrasonic probes were fabricated based on the multilayered microfabrication approach. The arrayed transducers were made of 1-3 piezoelectric composites and backing layers to improve the axial resolution (FIG. 14). We used the silicone elastomer with a modulus of 69 kPa as the probe-skin interface, which ensured intimate contact between the transducers and skin, therefore enable gel-free acoustic sensing (FIG. 15). The gel-free probes showed high durability in tissue sensing over long-term. Our results suggested the sensor could survive repetitive use over six months and showed negligible performance degradation (FIG. 16).

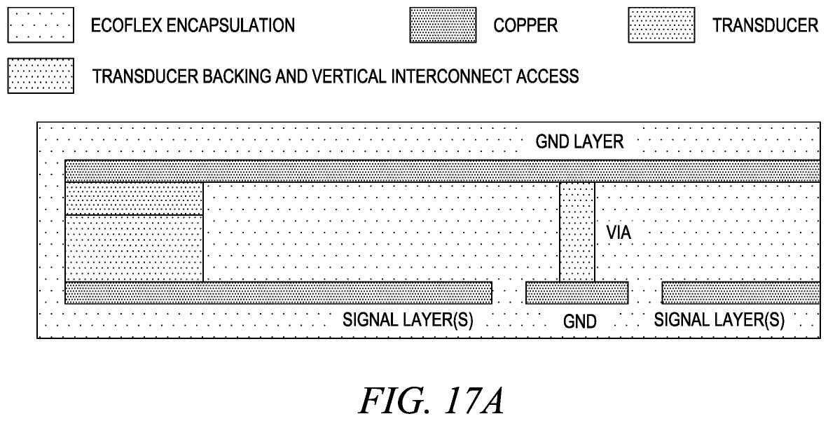

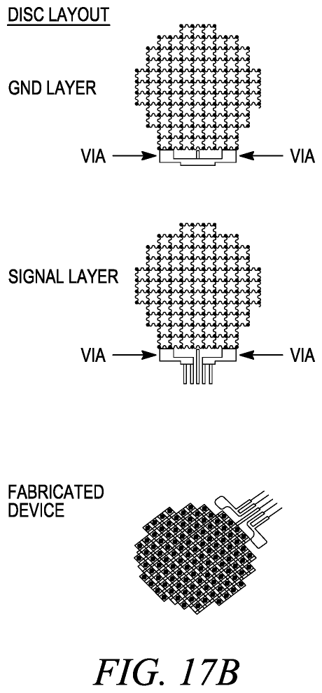

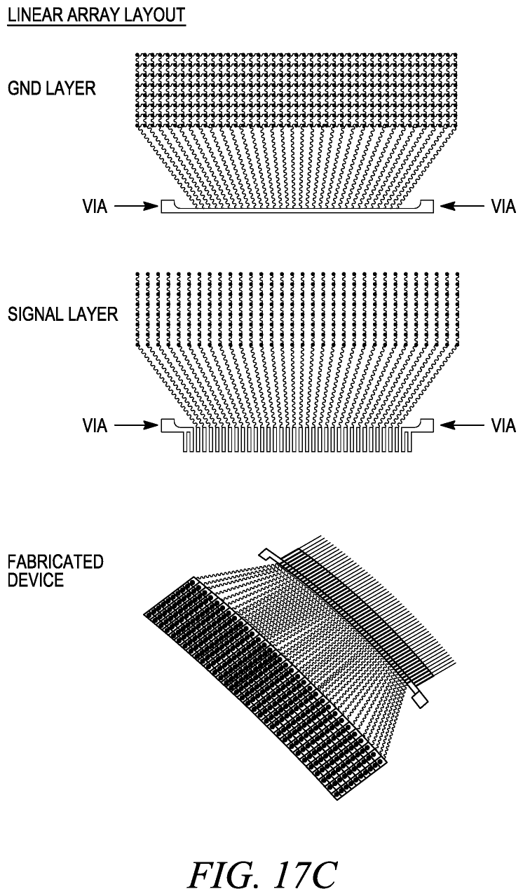

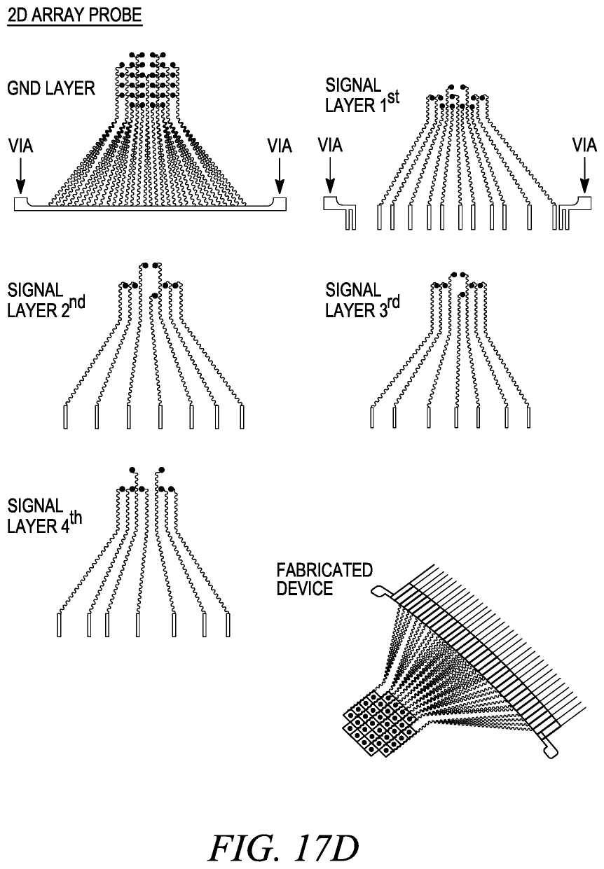

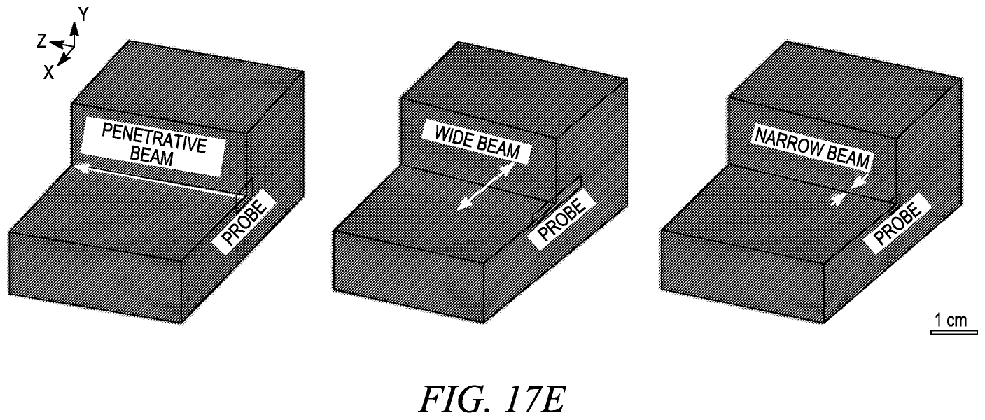

For probe fabrication, we sandwiched the transducers with copper serpentine interconnects prepared by laser ablation and transfer printing. The serpentine interconnects help achieve the stretchability of the transducer array. Vertical interconnect accesses were added to connect the ground electrodes and signal electrodes in different layers. The entire structure was encapsulated by silicone elastomer (FIG. 17a).

There were three probe layout designs, including a disc, a linear array, and a 2D array (FIG. 17b-d). These layout designs were simulated to confirm their transmission characteristics, where distinct beam patterns and aperture coverages were illustrated (FIG. 17e).

For the disc, 112 piezoelectric transducers at 2 MHz were used. All of these transducers were arranged within a circular region and connected in parallel, functioning as a single transducer for high transmission intensity. Such a design resulted in a highly penetrative transmission beam (FIG. 17e left), which was suitable for sensing deep organs (e.g., heart and diaphragm).

For the linear array, 256 transducers at 4 MHz were arranged with a bi-axial pitch of 0.8 mm. 8 transducers in the same column were connected in parallel to enhance the transmission intensity. 32 such columns constituted the linear array, yielding a 25.4 mm ultrasonographic aperture at moderate penetration depth (FIG. 17e middle), which was suitable for sensing central arteries (e.g., carotid artery, femoral artery, and abdominal aorta).

For the 2D array, 32 transducers at 6 MHz were used to constitute the array with a 0.8 mm bi-axial pitch. The overall dimension of the 2D array was the smallest in comparison with the other two cases. Such a design guaranteed a narrow beam (FIG. 17e right), which allowed for high spatial resolution sensing for shallow (e.g., radial and brachial) arteries.

### Sequence Control of the Ultrasonic Sensing

To achieve ultrasonic sensing, we customized the control sequence of the USoP, as shown by the detailed flow diagram (FIG. 6a). Each operation cycle of the USoP was divided into the pulse-echo sensing period and the multiplexing period. The switching between these two periods was controlled by the sequencer toggling the receive-enable signal (FIG. 6b).

In the pulse-echo sensing period, the receive-enable voltage was set to be logical high for 320 μs. Within this period, the microcontroller sent trigger signals to allow the pulse generator to output a high-voltage impulse, and the receiver circuit then received the echo signals from the transducer (FIG. 6c).

In the transducer multiplexing period, the sensing-enable voltage was set to be logical low for 680 μs. Within this period, the sequencer sent a series of digital signals to the multiplexer, including the clock (CLK), reset (RES), digital input (Dᵢₙ), and latch enable (LE). These digital signals functionalized the shift register and latch in the multiplexer for transducer selection. An example channel selection sequence was shown in FIG. 6d. A RES signal was first applied to the latch to clear previous channel selection, and then the Dᵢₙ was turned to logical high to initiate channel selection. Three rising edges were counted before LE signal turned low to latch the channel selection. Therefore, the third sensing channel was selected for the next cycle of pulse-echo sensing.

### Multi-Mode Ultrasonic Sensing

Some embodiments of the USoP are designed to support multiple ultrasound sensing modes, including amplitude mode (A-mode), motion mode (M-mode), and brightness mode (B-mode).

A-mode is a fundamental sensing mode where the ultrasonic probe interrogates the tissue as a one-dimensional depth recorder and produces a graph of the echo amplitude against the acoustic time-of-flight. An ultrasound beam was generated to penetrate the tissue layers, and then the beam was reflected by tissue interfaces of mismatched acoustic impedances. The tissue impedance information was then encoded in the amplitudes of the ultrasonic reflections, while the depth information was encoded in the acoustic time-of-flight. An example of A-mode sensing is shown by the arterial diameter measurement using a 4 MHz probe (FIG. 27a left). The posterior and anterior wall reflections were captured as the local maximums in the echo amplitude. Based on the echo amplitude signal, the arterial diameter could be calculated from the acoustic time-of-flight and acoustic speed in tissues (7 FIG. 27a right).

M-mode can be considered as continuous A-mode sensing. In M-mode, the echo amplitude is instead encoded as the brightness of the pixel, freeing up one axis of the graph for temporal information. Therefore, M-mode can capture the motion of tissue interfaces over time along a one-dimensional scanning line, providing sensing resolution in depth (y-axis) and in temporal domains (x-axis). In M-mode, the ultrasonic beams were repetitively transmitted to tissues for continuous sampling. During each cycle of transmission, one frame of A-mode signal was generated. By converting the A-mode frames into grey-scale pixels columns and plotting these columns as a function of time, M-mode images could be generated. An exemplary application capturing the carotid artery pulsation suggests that M-mode images can continuously capture the arterial distensions using a 4 MHz linear array. Two frames of radiofrequency echo signal show the minimum and maximum arterial diameters (FIG. 27b left), which correspond to the diastolic and systolic phases of the arterial pulsation (FIG. 27b right).

Moreover, when a probe with 2D layout is used in M-mode sensing, not only the axial resolution but also the spatial distribution of the motion can be acquired. Each transducer in the 2D array can generate an independent beam for M-mode sensing, and the amplitude of tissue movements was then calculated to locate the position of maximum motion amplitude. Such a sensing mode can be used for spatial detection of target arteries or guiding catheterization. As a demonstration, we mapped the arterial pulse waveform at the brachium using a 6 MHz 2D layout probe. The arterial pulse amplitudes and the mapped location of the brachial artery are shown in FIG. 27c.

Besides axial resolution, the lateral and elevational resolutions of the arrayed probes could be defined by the transmission beam patterns in A-mode and M-mode. Ideally, a single transducer would transmit a narrow beam. However, the real beam would spread laterally and elevationally. With such a spread beam pattern, two adjacent objects with a spacing smaller than the beam width cannot be differentiated by the transducer. Thus, this beam width determines the lateral and elevational resolution of non-imaging sensing. Therefore, we simulated the transmission beam patterns, and characterized the −3 dB width of the beam as the lateral/elevational resolution of three probes (7 FIG. 28).

B-mode generates images with axial and lateral resolutions, while the elevational resolution is also defined by the transmission beam pattern. In B-mode, arrayed transducers sequentially transmit and receive echo signals, working as a synthetic active aperture. The received echo signals are processed by delay and sum beamforming and I/Q filters, and then the echo amplitudes are converted to pixel brightness to reconstruct grey-scale 2D images. To demonstrate the B-mode sensing resolution of the 4 MHz linear array, we used a phantom made of an iron wire in water (FIG. 27d left). We defined the imaging resolution as the full width of the half maximum of the echo from the iron wire. when the iron wire was moved from 1 cm to 3 cm in depth, the axial and lateral resolution degraded, from 0.99 mm to 2.50 mm and from 0.75 mm to 2.5 mm, respectively, (FIG. 27d right).

### The Sensing Stability Under Probe Deformation

The soft probes that conform to highly curved skin surfaces may experience phase distortion. Therefore, we characterized the image stability with array distortions in both elevational and azimuth planes.

The elevational distortion is not critical for either A-mode, M-mode applications, or B-mode imaging when the probe's elevational aperture is small, because the smaller the elevational aperture, the smaller the time delay error caused by array bending (FIG. 29a,b). We simulated the transmission beam patterns with varying bending radius (from 6 mm to ∞) (FIG. 29c). Although the beam patterns suggest bending may introduce undesired side lobes, the intensity of these lobes is much smaller than the main lobe (FIG. 29d). Additionally, when the bending curvature radius is >6 mm, the transmission beam pattern would have negligible widening (FIG. 29e). Considering typical body parts have surface curvature radii much larger than 6 mm, the elevational distortion induced by human studies could be neglected.

While the elevational distortion would not affect imaging applications, the azimuth distortion may compromise the B-mode imaging if the array deformation exceeds a safety threshold. Because beamforming requires accurate positioning of each transducer in the array to calculate the delay function, a bent array would cause phase aberration and resolution degradation. We simulated the B-mode images of point sources to quantify the effect of bending curvature on the images (FIG. 30). With the bending curvature radii <6 cm, the B-mode images show artifacts in the shallow area (FIG. 30b, upper panels). When the bending curvature radii >6 cm, the imaging quality is acceptable without obvious artifacts (FIG. 30b, lower panels). Considering most body surfaces have curvature radii larger than 6 cm, the imaging results could be reliable.

### Measurements of Tissue Interfacial Motions

The motion of tissue interfaces can be continuously captured using M-mode sensing. By transmitting ultrasound beams into tissues at a pulse-repetitive-frequency of 25 Hz˜1 kHz, the displacement of various dynamic tissue interfaces can be interrogated. Displacement of the tissue interfaces is encoded in radiofrequency echo signals.

To decode the tissue motions, an auto-correlation method was deployed. In consecutively collected radiofrequency data frames, the echo from a tissue interface constantly moves within a specific range, shifting along the time axis but roughly maintaining its profile (FIG. 31a).

To decode the motion amplitude, the ultrasound radiofrequency data were first segmented to exclude the signal without motion. Envelopes of the segmented signals were then generated. After that, the auto-correlation method was applied to the generated envelope to obtain the auto-correlation value between adjacent frames (FIG. 31b). The lag (t) between two adjacent frames could then be determined by the position of the maximum auto-correlation value (FIG. 31c). The motion, also known as the displacement between two frames, was calculated as half of the acoustic round trip d=c×t/2. Noted that the auto-correlation decoding is based on envelope shifting, thus it is not sensitive to the transducer bandwidth or ringing in the radiofrequency signals as long as the envelope can roughly maintain its profile during shifting.

The tissue interfaces in this study, such as arterial pulsation, cardiac contraction, and diaphragmic movement, were of varying depths and excursion amplitudes, as summarized in Table 2.

Therefore, a proper selection of ultrasonic probes was needed to fit the specific sensing depths and resolutions. The waveforms in FIG. 2a were collected from a healthy 25-year-old participant. In these measurements, a 6 MHz 2D probe was used for arterial pulsations in shallow arteries with minimum excursions (˜0.05 mm), such as the radial (2 mm deep) and brachial arteries (4 mm deep). A 4 MHz linear array probe was used for deeper arteries with medium excursions (˜0.5 mm), such as the carotid artery (14 mm deep), femoral arteries (17 mm deep), and abdominal aorta (60 mm deep). A 2 MHz disc probe was used for central organs with large excursions (>8 mm), such as the heart (70 mm deep) and diaphragm (120 mm deep).

### Measurement and Calibration of Arterial Blood Pressure

From biomechanics, the measured pulse intensity effectively represents the arterial diameter change, which is a function of two variables: blood pressure and arterial stiffness. The blood pressure tends to expand the cross-section of the artery, while the arterial wall stiffness resists this expansion.

The exponential relationship between the diameter and arterial stiffness is independent of the blood pressure at the time of measurement within the physiological range (63-200 mmHg). The equation can be used to derive:

$$p ( t ) = {p}_{d} * {e}^{β ( \frac{D ( t )}{{D}_{d}} - 1 )}$$

$$And$$

$$β = \frac{{D}_{d} ln ( {p}_{s} / {p}_{d} )}{{D}_{s} - {D}_{d}}$$

where p(t) is the time-dependent blood pressure and D(t) is the time-dependent arterial diameter; Dₛ and D_{d} are the systolic and diastolic arterial diameters, respectively, derived from the measured pulse intensity; pₛ and p_{d} are the reference systolic and diastolic pressures, respectively, measured using a commercial blood pressure cuff; and is the stiffness index.

First, Dₛ, D_{d}, pₛ, and p_{d} at the brachial artery of the subject were measured to obtain with the subject sitting upright in a chair with the measured arm relaxed on a table. Specifically, pₛ and p_{d} were measured using a commercial cuff as calibration. The arterial diameter was then measured at the same location using the USoP to derive Dₛ and D_{d}. Then, p(t) was determined based on the corresponding D(t) measured by the USoP.

Measurement of p(t) using the USoP is highly stable with little need for recalibration. The initial calibration using the commercial cuff only needs to be performed once at the beginning of this process, as p_{d} remains relatively stable from beat to beat. The measurement of blood pressure using the USoP at the brachial artery is applicable to other arterial sites as well because and p_{d} do not change significantly along the major branches of the arterial tree. This allows us to equate brachial blood pressure measurements to the carotid blood pressure in healthy adults. Note that and p_{d} may change substantially on younger subjects and patients with vascular diseases, such as carotid atherosclerosis. In these populations, we may need to acquire accurate local carotid stiffness index and carotid blood pressure using catheterization to minimize the calibration error. In addition, the body habitus of the subject may also influence the calibration accuracy. For example, the height of subject may influence vascular resistance and further influence blood pressure calibration. In such cases, the vascular resistance could be estimated using nomograms or demographic databases, and then the stiffness index for blood pressure calibration could be corrected for better accuracy.

### Pulse Wave Velocity Measurements

The pulse wave velocity is defined as the propagation distance divided by the pulse transit time. Following a standard procedure, the propagation distances were measured on the body surface of the participants using a tape measure. Example tape measurements from a healthy participant illustrate the path lengths (FIG. 9a). Then, a pair of USoPs were deployed to measure the pulse propagation delay between myocardium contraction waveforms and the arterial pulse waveforms (FIG. 9b). For each measurement pair, the two USoPs were synchronized by encoding time stamps in each cycle of pulse-echo transceiving.

Following the recommendations for pulse wave velocity measurement from the ARTERY Society, the pulse transit time was calculated based on the foot-to-foot method, where the pulse transit time was defined as the mechanical propagation delay between the diastolic phase of myocardial contraction and arterial pulsation waveforms (FIG. 9b). To validate the accuracy of the pulse transit time, the results of the USoP were compared with those of the tonometer. The comparison suggests a mean difference of <1 ms, showing high consistency between the two devices (FIG. 9c).

A systemic stiffness mapping across different arterial segments was performed to show the variation of pulse transit time and, therefore, regional pulse wave velocity (FIG. 9d). We observed an apparent increase in pulse wave velocity, indicating an increase in arterial stiffness, from heart-proximal (e.g., heart-aorta, heart-carotid artery, and heart-femoral artery) to heart-distal branches (e.g., heart-brachial artery and brachial-radial artery) (FIG. 9e). A cold pressor test was performed sequentially. After the subject's hand was put in ice water for 5 min, the pulse wave velocity remained almost stable at proximal branches (e.g., heart-aorta, heart-carotid artery, and heart-femoral artery), but increased substantially at distal branches (e.g., heart-brachial artery and brachial-radial artery) due to the cold-induced regional vasoconstriction (FIG. 9e).

### Evaluation of Respiratory Function Based on Typical Expiratory Volumes

According to the guidelines from American Thoracic Society and European Respiratory Society for respiratory function testing, we measured the typical expiratory volumes such as the forced vital capacity (FVC) and forced expiratory volume in one second (FEV₁) (Table 3).

A lower limit of normal (LLN) was used as the diagnostic threshold. The LLN was set as each parameter's value of the lower fifth percentile of a large healthy reference group. The LLN depends on the age, height, ethnicity, and other health conditions of the subject, so its value vanes in different individuals. In practice, the LLN values for a specific subject were calculated using the NHANES III database provided by the Centers of Disease Control and Prevention.

Then, the respiratory function was evaluated based on the following criteria: If FEV₁/FVC ratio <LLN, the patient is considered to have an Obstructive issue. if FEV₁/FVC ratio LLN while FVC ≤LLN, the patient is considered to have a restrictive issue. Further assessment should be made according to the patient's total lung capacity. If FEV₁/FVC≥LLN and FVC≥LLN, the patient is considered healthy.

In this study, the FVC and FEV₁ were derived from the USoP measured diaphragm excursion (FIG. 34a). A four-quadrant plot shows the measurement results (FIG. 34b). Data points in the top-right, top-left, bottom-right, and bottom-left suggest that the patient has healthy, obstructed, restricted, and combined obstructed and restricted conditions, respectively. For a health subject without respiratory issues, these values could be used to quantify expiratory performance.

A longitudinal study was performed to record the FVC and FEV₁ of a participant. The initial FVC and FEV₁ values were recorded, and then the participant was enrolled in a training program to perform regular aerobic exercise for four months. A significant increase in the FVC was observed from the four-quadrant plot (FIG. 34b), suggesting improved respiratory function post-training.

Performance Validation of Deep Learning Models and Comparison with Logistic Models

1. Performance Comparison Between Available Deep Learning Models

We compared the performance of four different models, including MobileNetV2, ResNet, VGG11, and VGG13, in the carotid artery classification task. The model performance was determined through a leave-one-out 10-fold training-validation process. Specifically, 4600 images were randomly divided into ten folds; each with 460 images. In each turn, we picked one-fold in order as the validation set and the remaining nine folds as the training set. After ten turns, we calculated the average performance of each model.

Based on the training-validation results, we generated the receiver operating characteristic curves and evaluated the models by the area under the curve. Each point on the receiver operating characteristic curves represents the true positive rate and false positive rate under different classification thresholds from 0 to 1. VGG13 with batch normalization achieved the highest area under curve and accuracy (FIG. 10) and thus was selected as the best model for this work.

2. Dependability of the VGG13 Model

To validate the model dependability and prove that the VGG13 model is truly learning the arterial pulsating pattern for classification rather than building spurious correlations between training sets and validation sets. We trained and validated the VGG13 model with images that the artery region partially and totally cropped out (FIG. 35a, left three panels). With the salient regions removed, the remaining images lose rich geometrical information including bright strip patterns (strong ultrasonic reflection from arterial walls) and sawtooth texture (arterial pulsating). Therefore, the trained classifier is supposed to degrade in performance.

As shown in FIG. 35b, the VGG13 model performance experienced a gradual degradation with more salient regions cropped. Note that even with the two walls cropped, the VGG13 model maintained its classification ability and performed better than random guesses (50% accuracy). For one wall-cropped case, the remaining posterior wall is still an identifiable feature for classification. For the two-wall cropped case, the pulsating feature also existed in the surrounding tissue. When the artery pulses, the mechanical cave would propagate in surrounding tissues and generate tissue pulses, although the tissue pulses had smaller amplitudes due to energy loss in propagation. Therefore, the tissue texture (FIG. 35a, the third panel from left) could also serve as a differentiable but weak feature.

In addition, we did an additional experiment to shuffle the label before the train/validation split happens. The images were labeled with CA and nCA regardless of their true identity (FIG. 35a, the rightmost panel). After training, the model learned chaotic correlations and had a poor performance that the precision, recall, and accuracy are close to 50% (FIG. 35b). Differently from regional cropping, randomly labeled images failed to guide the model to generate an efficient classifier to differentiate CA and nCA images and resulted in unpredictable and poor classification results.

3. Advantage of VGG13 Model Over Conventional Logistic Models

Besides the deep learning classification model, we also developed a logistic classification model based on carotid artery image features. We intuitively chose the sawtooth-shaped pattern in the image as the most salient feature to differentiate carotid artery and non-carotid artery images. Based on conventional image processing methods, the model took three steps to classify images (FIG. 36a). First, we segmented the images to keep only the arterial region based on empirical knowledge of carotid artery depth (˜1.5 cm). Second, the edges of the gray-scale image were extracted (FIG. 36b). The image passed a Gaussian smoothing filter to remove excessive details and then the potential wall edges were extracted by a Canny detector. Third, the detected edges were combined (by averaging their vertical coordinate value) into one edge curve representing possible arterial pulses. We then detected the pulse through spectrum analysis. FIG. 36c shows an example of CA image, where the edge curve was extracted from a carotid artery image. After fast Fourier transforming, the frequency response suggested a peak at ˜1 Hz representing a heart rate of 60 bpm. In an nCA case, the extracted edge curve would be non-periodic, therefore its frequency response would show no notable peaks within the heart rate range. Therefore, by detecting peaks in the frequency spectrum, we could know whether real carotid pulses exist, therefore classify CA and nCA images. In our model, the heart rate range was set to 48-108 bpm.

Moreover, this logistic model could use either one-wall or two-wall detection criteria. For one-wall detection criteria, as long as there is one “pulsating wall” (most likely the anterior wall) detected in the image, the image is considered a “CA image”. The two-wall detection only considers the image to be “CA” if both anterior and posterior walls are present. With this more rigorous criterion, two-wall detection could reject more false negative (nCA) cases, but also reject more true positive (CA) cases. Our validation results supported the same conclusion that the one-wall criterion offered a better recall, while the two-wall criterion had a better precision. Two criteria performed similarly in accuracy, which reached ˜61% (FIG. 36d).

However, a classification accuracy of 61% was far from acceptable. In iterative tests, we found that the classifier tended to fail with perturbed images in this work (e.g., noise coupling, artery shifting, and artery missing). These corner cases could compromise the edge detection process (FIG. 36e) and eventually result in false classification. On the contrary, the VGG13 model could handle the perturbation in the images and maintain high accuracy (>99%) (FIG. 10). In addition, the critical parameters used in the logistic model (e.g., the Gaussian standard deviation and edge detection threshold) are subject-dependent. Manual iterations and tedious optimizations would be required before the model could accept a new subject. The deep learning model could transfer the model to new subjects via a minimal entropy correlation alignment model without manually tuning parameters.

With these results presented, we could conclude three advantages of the deep learning model over logistic models and justify the use of deep learning models in our task. First, it offered better classification accuracy. Second, it is more dependable to handle “corner cases” than the logistic models. Third, it offers labor-free generalization opportunities while the logistic models rely on manual optimizations.

Probability Profile Generation from the Prediction Results

Deep learning networks produce a posterior probability for the presence of the carotid artery in each of the 32 channels. Ideally, this should follow a bell-shaped profile, with the peak of this profile representing the arterial center. However, the probabilities produced by the network may have random noise due to possible acquisition of compromised M-mode images. This could lead to misjudging the position of the arterial center.

To decrease the possibility of such failure, we convolved the raw prediction profile with a one-dimensional Gaussian kernel function. In our experiments, this was sufficient to produce a bell-shaped curve that reliably determines the position of the arterial center. The plot of FIG. 37 shows 50 predictions of the carotid artery center against the human-determined ground truth, suggesting a close to one-to-one correspondence (y=1.004x-0.137) between the predicted channel number and the ground truth.

### The Limit of Motion Tolerance and Pulse Waveform Continuity

The speed of head motion is a critical factor that can compromise model prediction and waveform recording of the carotid artery. For very high motion speeds, attempted measurement of the carotid artery risks the signal passing through the sensing channels without even generating a full pulse cycle. Because the pulsation pattern in the M-mode image is the key to differentiating carotid from non-carotid artery images, the rapid motion might possibly result in a lack of features for the model to recognize. To address this possibility, we recorded the arterial signal with an increasing head yawing rate to demonstrate the robustness of the waveform acquisition and expected a classification model failure by increasing the yawing rate ultimately.

The head yawing rate was quantified using a pair of inertia measurement units (FIG. 38). When the head yawing rate was increased from 0°/s to 80°/s, the recorded pulse periods decreased from 2.8 s to 0.3 s (FIG. 41). The former period contained at least two cycles of arterial pulsation at a resting heart rate (i.e., 60-80 bpm), while the latter period contained less than ⅓ of a pulse cycle. Without a complete pulsation pattern in the M-mode image, the machine learning model was unable to recognize the carotid artery. According to the results in FIG. 41d, the threshold of a recognizable pulse cycle is ˜1 s, corresponding to ˜1 pulse cycle and a head yawing rate of ˜60°/s, to ensure the true positive (true carotid artery image) rate is high enough for a successful prediction.

At a relatively low yawing rate (i.e., <60°/s), each sensing channel can collect a long period of arterial pulses containing several cardiac cycles. In this situation, the classification model reliably recognized the M-mode images containing the carotid artery pulses. Thus, the pulse waveforms experienced no distortion under the re-selection of scanning channels. However, at a relatively high yawing rate (i.e., ≥60°/s), the artery crossed over sensing channels, resulting in a significantly decreased pulse period in M-mode images and thus a low true positive rate. Ultimately, the waveform recording experienced distortion.

After the rapid motion, the model can continue searching among sensing channels, and whenever a channel has a ˜1 s pulse period recorded, the model is then able to recognize this latest best channel and establish a new scanning channel. Thus, good pulse waveform recording can be quickly restored (FIG. 42).

### Training Principles of a Minimal Entropy Correlation Alignment (MECA) Model

Training classifiers require data labeling, which requires some effort by human annotators. Domain adaptation is used to transfer a classifier trained with labeled data from a single subject to other subjects for whom labels are not available. We define the training set as the source domain data, [symbol] ₛ={(xᵢ^{s}, yᵢ^{s})}ᵢ₌₁ⁿ^{ₛ}, containing pairs of images xᵢ^{s} and labels yᵢ^{s}. The images collected from new subjects belong to the target domain, [symbol] ₜ={xᵢ^{t}}ᵢ₌₁ⁿ^{ₜ}, where we only have images, xᵢ^{t}, but no labels, yᵢ^{t}.

The goal of domain adaptation is to learn a transfer function G that aligns features extracted from images from the source ( [symbol] ₛ) and target ( [symbol] ₜ) domain. We select the MECA as our domain adaptation model because it provides a systematical way to adjust the weight of the domain discrepancy and the cross-entropy in the loss function. It is crucial to minimize the human effort in hyper-parameter fine-tuning for applications in this work because there will be multiple subjects. In this model, the distance between the domains is measured with the squared log-Euclidean distance, which is defined as:

$${l}_{log} ( {C}_{G ( {𝒟}_{s} )} ) , {C}_{G ( {𝒟}_{t} )} ) = \frac{1}{4 {d}^{2}} {‖ Udiag ( log ( {σ}_{1} ) , … , log ( {σ}_{d} ) ) {U}^{T} - Vdiag ( log ( {μ}_{1} ) , … , log ( {μ}_{d} ) ) {V}^{T} ‖}_{F}^{2}$$

where [symbol] and [symbol] are the covariance matrices of the feature vectors generated by the domain transferer G for source and target data, respectively; d is the dimension of these feature vectors; U and V are the eigenvector matrices of the eigendecomposition of [symbol] and [symbol] ; σ and μ are the corresponding eigenvalues; and F represents the Frobenius norm. By minimizing this distance, we can train the transfer function G to unify the source domain and the target domain.

Dataset Size Required For Domain Adaptation

To Verify the Minimal Number of Images that were Needed for a Successful domain adaptation, we performed a grid search on the number of training images (labeled) and new images (from a new subject, unlabeled). For this, we reduced the number of training images from 256 to 32 with a step of 1, and the number of new images from 256 to 16 with a step of 16. A heatmap of the resulting classification accuracy is shown in FIG. 34. We found that 67 labeled images from an existing subject and 32 unlabeled images from a new subject were sufficient to achieve an accuracy above 90%. This could be considered a minor effort in image collection. When the number of images drops below these boundaries, the accuracy can drop significantly (FIG. 44).

### Systolic and Diastolic Blood Pressure Changes During Exercise

The acute increase in systolic blood pressure during exercise is primarily driven by increases in cardiac output, while the change in diastolic pressure during exercise is additionally affected by peripheral vascular resistance. During exercise, the cardiac output increases while the peripheral vascular resistance decreases, counterbalancing the changes to diastolic pressure by dissipating the pressure across the vasculature. These interactions manifest as greater increases in systolic pressure than in diastolic pressure during exercise.

### Quantifying the Vascular Response to Exercise

In both cycling and HIIT, the blood pressure waveforms have changing profiles, suggesting increased differences between the systolic peak and secondary (reflected) peak during exercise (FIG. 45). This change indicates a reduced reflection from the distal ends of the arterial tree due to flow-mediated vasodilation.

We used the pulse wave decomposition analysis method to analyze the pulse profiles and quantify the vasodilation occurring in exercise. Using this method, the pulse waveforms measured from central arteries (e.g., aorta and carotid artery) are decomposed into the forward and reflection waves. The forward waves are generated by the heart, while the reflection waves are considered to be backpropagations from the distal ends of the arterial tree (FIG. 46a). More constrictive arteries are of higher impedance and tend to have stronger reflection waves and faster backpropagation speeds (FIG. 46b upper panel). This results in an early and strong reflection peak in the arterial pulse waveform. On the contrary, dilated arteries are of lower impedance, which have weaker reflections and slower backpropagation speeds, and thus, lead to a late and mild reflection peak in the pulse waveform.

We used the AIx to quantify vasodilation. The AIx is defined as the difference between the systolic peak and the reflection peak/inflection point divided by the systolic peak. Example waveforms recorded before and after exercise indicate an increase in the AIx due to dilated arteries and decreased impedance of pulse wave propagation post-exercise (FIG. 46b lower panel).

In practice, the AIx can be calculated in a beat-to-beat manner from the blood pressure waveforms. In this work, the beat-to-beat AIx's were averaged over every minute to minimize potential errors associated with accidental waveform distortions.

### Changes in Arterial Stiffness Index and Errors in Blood Pressure Calibration During Exercise

The blood pressure-arterial diameter relationship is applicable to exercising subjects. The β-stiffness index is independent of the blood pressure in the physiological range. Also, it has been reported that there are no significant changes in arterial stiffness before and after non-resistance exercise, such as cycling or HIIT, in elastic major arteries (e.g., aorta and carotid artery).

To quantify the error in blood pressure recording during exercise, we compared β values during and after cycling (FIG. 47a). The carotid artery diameter during strenuous exercises increased up to 19.91% from baseline. Accordingly, the maximum blood pressure error is calculated to be 1.58 mmHg between the two β values from the resting carotid artery diameter (3.92 mm) to the high intensity exercise-induced carotid artery diameter (4.70 mm) (FIG. 47b). This blood pressure error was lower than the recommended maximum mean difference of 5 mmHg by the Association for the Advancement of Medical Instrumentation. Thus, there is no need to adjust β when measuring blood pressure during exercise.

### Stroke Volume Estimation Using the Pulse Contour Method

In the Windkessel model of the circulation, the blood pressure waveform can be used to monitor fluid flow throughout the circulatory system, such as flow velocity, distensibility, pressure, and volume, which allows relating the pulse contour waveform to the stroke volume.

In the Windkessel model, the distensibility c is expressed as:

$$c = \frac{dP}{dV} = c$$

where P is pressure and V is the volume of the fluid. The main differential equation describing the system is written as:

$$i * dt = \frac{dP}{c} + \frac{P * dt}{w}$$

$$or$$

$$dt = \frac{dP}{c ( i - \frac{P}{w} )}$$

where i is the volume of liquid flowing in per unit time; t is time; and w is the constant

$$\frac{8 L μ}{π {r}^{4}}$$

from Poiseuille's law.

Because the artery is nonrigid, the inflow and outflow at a given time are not equal to each other even though the blood is an incompressible fluid. Therefore, i should be averaged over the entire cardiac cycle. Integrating the main differential equation leads to:

$$t = - [ \frac{w}{c} ( i - \frac{P}{w} ) ] + [ \frac{w}{c} ( i - \frac{{P}_{0}}{w} ) ]$$

for a nonzero initial pressure P₀ at time t=0. The equation then becomes:

$$t = \frac{w}{c} ( \frac{i - \frac{{P}_{0}}{w}}{i - \frac{P}{w}} )$$

leading to the pressure equation:

$$P = w ( i - \frac{i - \frac{{P}_{0}}{w}}{{e}^{\frac{tc}{w}}} )$$

Wesseling and coworkers have used the aforementioned Windkessel model as a basis for calculating the stroke volume by integrating the area under the curve of the pulse contour. In essence, the pressure increases in proximal large arteries (e.g., aorta or carotid) are determined by the systolic blood output from the heart. Therefore the area under the systolic portion is proportionally related to the stroke volume, by a factor representing the characteristic impedance of the circulatory system, Z:

$$Stroke Volume = \frac{1}{Z} {\int }_{0}^{{T}_{e}} [ P ( t ) - {P}_{d} ] dt$$

where Tₑ is the end of the ejection period; P(t) is the real-time blood pressure; and P_{d} is the diastolic pressure. The characteristic impedance Z may be calibrated to another measure of stroke volume such as indicator dilution, or simply estimated using factors such as age, sex, height, and weight of the subject. In this study, we adopted an estimated value for the participant's characteristic impedance Z=0.056 mmHg·s/ml.

### Errors in Conventional Ultrasonography

Errors can be generated in conventional ultrasonography on both the operator side as well as the patient side. On the operator side, reliable probe positioning and accurate scanning are critical (FIG. 50a-c). On the patient side, during the examination procedures, the measured body part must be still to avoid motion artifacts (FIG. 50d). However, neither operator skills nor subject compliance is necessarily accessible outside the hospital or healthcare environment. Thus, enabling ultrasonography to be used by a general user on a moving subject during examination represents a critical step forward in the development of point-of-care ultrasound technologies.

### Clinical Benefits of Continuous Monitoring During Exercise

First, continuous monitoring of blood pressure has stronger prognostic values than single transient measurements. Monitoring the blood pressure in response to stressors—most potently exercise—for an exaggerated systolic response is independently predictive of cardiovascular mortality and risks, including future hypertension, stroke, atherosclerosis, cardiovascular abnormalities, insulin resistance, and hypercholesterolemia. Other stressors such as mental stress have similar associations, but due to their long-lasting or unpredictable nature, may require continuous monitoring over days or weeks in order to capture.

Second, vascular response to exercise, as a valuable indicator of cardiovascular fitness, can be characterized by pulse waveform analysis. For example, the AIx reveals pulse wave reflection and arterial stiffness. A low AIx is desirable, as high arterial stiffness is strongly associated with cardiovascular diseases. Increased arterial stiffness produces additional systolic load on the heart, limiting the exercise cardiac output and forcing the heart to work harder, which may eventually lead to heart failure. Thus, reducing arterial stiffness is one of the main desired outcomes of endurance exercise training.

Third, cardiac function, such as stroke volume and resulting cardiac output which represents the heart's capacity to deliver blood throughout the body, can be derived using the pulse contour method. All cells in the body require oxygen and nutrients delivered via the blood for their metabolism. The inability of the heart to deliver sufficient blood to support the body's metabolic needs, such as abnormally low stroke volume and cardiac output at rest or early plateaus of cardiac output during exercise, is a hallmark of heart failure.

Fourth, for healthy populations, the same dose of exercise can result in very different responses in different persons (e.g., an average person vs. an athlete). Conventional measures of exercise intensity based on duration and repetitions are not personalized. The USoP can measure cardiovascular responses to exercise in real-time and thus provide insight into the actual workout intensity exerted by each person, which can guide the formulation of personalized training plans.

Fifth, for patient populations with cardiovascular disease, engaging in exercise is important for condition management. Exercise exceeding safety thresholds may induce risks, such as exercise-induced hypertension or cardiac arrest. The magnitude of the exercise-induced systolic blood pressure increase has also been shown to be predictive of mortality, making exercise measurements a valuable prognostic indicator. In addition, central diastolic blood pressure is one of the main elements driving coronary perfusion. Therefore, continuously monitoring the central diastolic blood pressure may provide an early warning signal for acute cardiac ischemia.

### Clinical Need for Continuous Tissue Monitoring in High-Risk Populations

The USoP can monitor the cardiovascular and respiratory systems autonomously, using similar image-based machine learning algorithms to those for arteries. Continuous monitoring of these vital systems can be critical for certain high-risk populations, yielding better patient management and clinical outcomes.

For example, senior populations are at high risk for developing coronary heart disease. However, the development of such diseases is chronic and often ignored before acute symptoms are detected (e.g., cardiogenic shock due to myocardial infarction). Continuous monitoring can detect reduced fractional shortening or abnormal ventricular wall motion that reveals degraded cardiac function. Therefore, early signs of coronary artery diseases can be identified, making timely management of the disease possible. Similarly, continuous monitoring of respiratory function can enable the early identification of pulmonary dysfunction, such as reduced expiratory volume, and provide early warning of acute processes (e.g., pneumonia) or more chronic pulmonary disease, allowing for earlier and more definitive interventions.

### Illustrative Methods

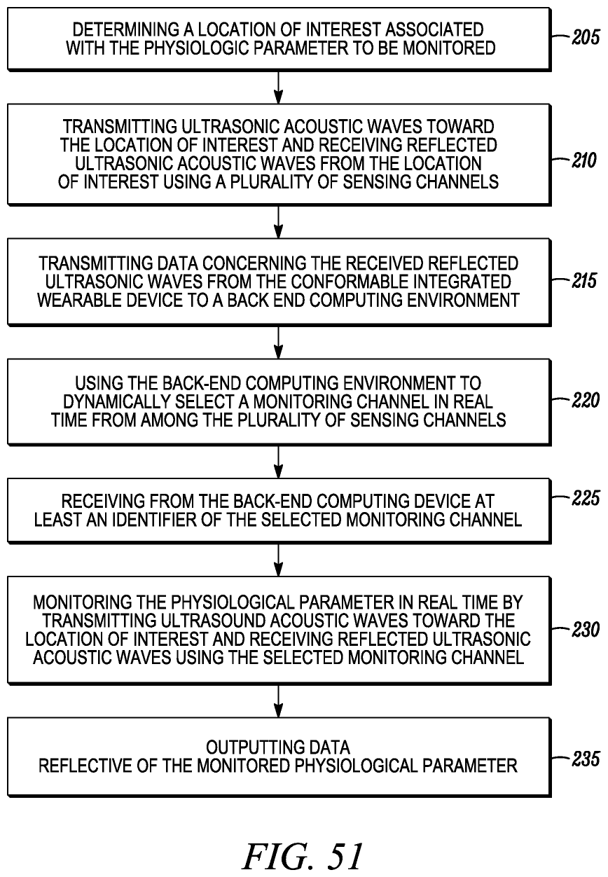

FIG. 51 is a flowchart illustrating one example of a method for monitoring a physiologic parameter that may be performed by various embodiments of the USoP described herein. In step 205 a location of interest associated with the physiologic parameter to be monitored is determined. Next, in step 210 ultrasonic acoustic waves are transmitted by a conformable integrated wearable device toward the location of interest and reflected ultrasonic acoustic waves are received from the location of interest using a plurality of sensing channels. Data concerning the reflected ultrasonic acoustic waves are transmitted by the conformable integrated wearable device to a back-end computing environment in step 215. In step 220 a monitoring channel is dynamically selected in real-time from among the plurality of sensing channels by the back-end computing environment. The conformable integrated wearable device receives at least an identifier of the monitoring channel from the back-end computing environment in step 225. In step 230 the conformable integrated wearable device monitors the physiological parameter in real-time by transmitting ultrasound acoustic waves toward the location of interest and receiving reflected ultrasonic acoustic waves using the selected monitoring channel. Data reflective of the monitored physiological parameter is output in step 235.

In the method depicted in FIG. 51 the monitoring channel is dynamically selected by a back-end computing, environment that is located external to the conformable integrated wearable device. In other embodiments, however, the dynamic selection of the monitoring, channel may be performed on the conformable integrated wearable device itself, thus avoiding the need to respectively transmit and receive data to and from the back-end computing environment in steps 215 and 225.

Also in the method of FIG. 51 the ultrasonic acoustic waves are transmitted to and reflected from the location of interest. in other embodiments, however, the ultrasonic waves that are transmitted to the location of interested pass through the location of interest and it is these resulting ultrasonic waves that are received an analyzed.

### Figure Descriptions

FIGS. 1a-1d provide an overview of the fully integrated USoP. a, A photograph of the encapsulated USoP laminated on the chest for measuring cardiac activity via the parasternal window. The inset shows the folded USoP. b, Design of the USoP, including a stretchable ultrasonic probe, a flexible control circuit, and a battery. The ultrasonic probe consists of a piezoelectric transducer array, serpentine interconnects, and an anisotropic conductive film (ACF) (upper left). The exploded view of the circuit shows two parts: (1) an AFE, including a 32-channel multiplexer (Mux), a transmit/receive switch (T/R SW), a receiver with a variable gain amplifier (VGA) and a filter, a pulse generator with a transmit controller (Tx ctrl) and a booster, and a sequencer; and (2) a DAQ module including a microcontroller unit (MCU) with a built-in analog-to-digital converter (ADC) and a Wi-Fi chip. The two modules are connected by serpentine electrodes, which allow the entire circuit to be folded for a smaller footprint. The circuit is powered by a commercial lithium-polymer battery. A smartphone application is designed to display the data stream from the USoP. From the ultrasonic data, M-mode images and physiological signals can be derived and displayed in real time. The smartphone can also communicate with a cloud server for further data analysis (lower right). c, Block diagram of the USoP showing the flow of analog impulse, analog echo, and digital signals. The AFE senses pulse-echo to generate ultrasonic signals, and the DAQ samples signals and wirelessly transmits the data to a terminal device for processing and display. d, B-mode imaging of the carotid artery (CA) and jugular vein (JV), while the subject is performing the Valsalva maneuver to dilate the JV (left). M-mode imaging of the pulsation pattern of CA walls (right).

FIGS. 2a-2e illustrate the monitoring and analysis of tissue interface motions using the USoP. a, Schematics and measurement results of seven representative dynamic tissue interfaces. b, Deriving physiological parameters from myocardial contraction. From the M-mode waveforms of the septum and left ventricular wall, the left ventricular internal diameter at end-diastole (LVIDd) and end-systole (LVIDs) can be used to derive the fractional shortening (left). Comparison of measurements between the USoP and a commercial ultrasound probe (right). The results are averaged from ten independent measurements, and the error bars represent the standard deviation (SD). c, Derivation of physiological parameters from the arterial pulse waveforms, including the heart rate and blood pressure. d, Bland-Altman plot showing measurement agreement between the USoP and a tonometer. For the heart rate, a mean difference of 0.013 beats per minute is observed, and 135 of 142 (95.1%) data points are within 95% limits of agreement defined by ±1.96 SD (left). For the blood pressure, a mean difference of 0.17 mmHg is observed, and 269 of 280 (96.1%) data points are within 95% limits of agreement defined by ±1.96 SD (right). e, Derivation of expiratory volume from the diaphragmatic excursion. Simultaneous measurements of diaphragmatic excursion and respiratory volume show a similar pattern (left). The regression on expiratory volume (V) with diaphragmatic depth (D) in normal breathing and forced breathing. Strong linear relationships, with correlation coefficients (CCs) close to 100%, can be found between the diaphragmatic excursion and expiratory volume in both breathing conditions (right).

FIGS. 3a-3f illustrate the autonomous and continuous blood pressure recording in a moving subject. a, Schematic cross-sectional view of a soft 4 MHz linear array sensing the carotid artery (left). Representative M-mode images of channels with beam penetrating or not-penetrating the carotid artery, classified as carotid artery (CA) or non-carotid artery (nCA) images, respectively (right). b, Flow diagram showing the process of autonomous CA detection and pulse waveform generation. c, Recording in a moving subject using the USoP with and without an autonomous algorithm. The algorithm can reliably track the CA position with head yawing from −80° to +80°, corresponding to a ˜19 mm CA displacement. Prediction scores of different transducer channels for tracking the CA at each yawing position and corresponding B-mode images collected by a commercial ultrasound machine (upper panel). By actively selecting the best channel to follow the CA motion (e.g., #5, #8, #16, #23, and #29), continuous pulse waveforms can be recorded. In contrast, without the auto-selection algorithm, a fixed channel (#16) results in signal loss during motion (lower panel). d, Two representative M-mode images recorded from the training subject (#1) and a new subject (#2), showing different image patterns (left). The histograms of the two CA images show a substantial difference in luminosity distribution (right). Inset: subject #2 has ˜six times more white pixels than subject #1, indicating thicker arterial walls. e, Schematic diagram showing the workflow of the minimal entropy correlation alignment model, consisting of two encoders with five convolutional (Conv.) layers and three fully connected (FC) layers. The classification loss and geodesic covariance loss are used to align features extracted from the training image set (source domain) and those from a new image set (target domain). f, Model generalizability validation on 10 subjects. The classification model is trained on each subject and validated on the remaining subjects. Without domain adaptation, the matrix plot shows an average classification accuracy of only 63.23% on new subjects (left). After domain adaptation, the average classification accuracy is boosted to 96.13%, showing the improved generalization of the classification model (right).

FIGS. 4a-4f illustrate the continuous monitoring process during exercise. a, Photographs showing a subject cycling while the carotid pulsation waveform is measured by the USoP with different head positions, including (i) forward, (ii) turned, (iii) bowed, and (iv) raised. The USoP can directly transmit data to backend system and display the result on the smartphone. Inertia measurement units are used to record the head motion. An automated cuff on the upper arm acquires brachial blood pressure levels for reference. b, Head motions recorded by the inertia measurement units during cycling. The carotid blood pressure waveforms and heart rate are recorded simultaneously using the USoP. The maximum increases in diastolic and systolic pressure are 17 mmHg and 45 mmHg, respectively. c, Zoomed-in view of the head motion, blood pressure waveforms, and heart rate recorded during the (i)-(iv) motion periods in b. The diastolic carotid pressures measured by the USoP agree well with the brachial pressures measured by the cuff. The systolic carotid pressures measured by the USoP are ˜10 mmHg lower than the cuff brachial values, due to lower distal reflections. d, Histograms of the diastolic and systolic pressures during cycling and HIIT. During cycling, the variations in diastolic and systolic pressures are 20 mmHg and 47 mmHg, respectively. During HIIT, the variations in diastolic and systolic pressures are 38 mmHg and 55 mmHg, respectively. e, Changes in augmentation indices during cycling and HIIT. The augmentation index first increases and then plateaus during cycling, and then recovers during resting (upper). The augmentation index fluctuates during HIIT, coinciding with the training-resting cycles (lower). Notably, the augmentation index was substantially higher during (ii) and (iv) training sessions, indicating greater arterial vasodilation. Average augmentation indices are calculated from fifty independent pulse waveforms every minute. The error bars represent the standard deviations of the recorded augmentation indices. f, Cardiac response to cycling and HIIT. In both exercise scenarios, the stroke volume first increases and then plateaus while the heart rate continues to increase. Cycling has a smaller increase in stroke volume than HIIT. The maximum cardiac output measured during HIIT is 15.6% greater than during cycling.

FIGS. 5a-5d presents data characterizing bandwidth, axial resolution, and penetration of the stretchable ultrasonic probes. a, Pulse-echo response and bandwidth of the probes with three frequencies. The full width at half maximum (FWHM) is labeled to show the axial resolution of each probe. The 2 MHz, 4 MHz, and 6 MHz can achieve 604 μm, 333 μm, and 229 μm resolution, respectively. Three probes could achieve a relative bandwidth of ˜50% to their center frequencies at −3 dB. b, The pulse-echo response of a commercial ultrasound probe with a center frequency of 3 MHz, which could achieve a relative bandwidth of 42.3%. c, Tissue targets to be sensed by the stretchable ultrasonic probe in this work. The 2 MHz probe is used for deep organ (heart and diaphragm) sensing. The 4 MHz probe is used for deep major artery (carotid, femoral, and abdominal aorta) sensing. The 6 MHz probe is used for shallow peripheral artery (radial and brachial) sensing. d, Transmission beam intensities as a function of penetration depth in tissue of the probes with different frequencies. The intensity decay was measured in water, and then converted into tissue decay with an attenuation factor of −0.3 dB/cm/MHz. Based on the penetration threshold of a −3 dB drop in intensity, the 2 MHz, 4 MHz, and 6 MHz can penetrate 164.0 mm, 77.7 mm, and 9.2 mm, respectively.

FIGS. 6a-6d shows schematics and the control sequence for ultrasonic sensing. a, Block diagram and signal transmission lines between the functional modules. The control circuit includes two functional parts: the AFE and the wireless DAQ module. The AFE consists of a multiplexer (Mux), a transmit/receive switch (T/R SW), a receiver, a sequencer, and a pulse generator. The DAQ module consists of a microcontroller (MCU) with on-chip analog-to-digital convertor (ADC), and a Wi-Fi transmitter. The dashed lines are for digital signal transmission and the solid lines are for analog signal transmission. b, The simulated control sequence for multiplexing and pulse-echo sensing, which shows the time sequence of the receive (Rx) enable, trigger, high-voltage (HV) pulse, clock (CLK), reset (RES), digital input (Dᵢₙ), and latch enable (L̅E̅) signals. c, Signals acquired by an oscilloscope showing the control sequence of the pulse-echo sensing and transducer multiplexing. d, Signals acquired by an oscilloscope showing the input sequence to the shift register for multiplexing and driving the transducer elements. All figure panels share the same color encoding scheme.

FIGS. 7a-7d illustrate deformation of the packaged USoP. a, 90° bending, b, 90° twisting, and c, 20% uniaxial stretching of the packaged USoP. d, A zoom-in view of the stretched interconnects.

FIGS. 8a-8g illustrate the skin integration of the conformal USoP device. The soft patch could conform to multiple curved body parts, including a, forearm, b, brachium, c, neck, d, lower chest, and e, abdomen. f-g, Skin integration of the device before and after exercise. The USoP could maintain robust adhesion to the skin after the subject performs intensive exercise and sweats.

FIGS. 9a-9d show pulse wave propagation paths and pulse wave velocity (PWV) measurements. a, Schematic illustration of the pulse wave propagation paths in this study. Five paths were investigated, including the heart to the abdominal aorta (H-Ao), the heart to the carotid artery (H-CA), the heart to the femoral artery (H-FA), the heart to the brachial artery (H-BA), and the brachial artery to the radial artery (BA-RA). b, Pulse waveforms collected by synchronized USoP pairs. The pulse transit time (PTT) was defined as the delay between the diastolic feet of the ventricular contraction and arterial pulses. c, The averaged PTT values by the USoP and the tonometer, showing consistency for both H-BA and BA-RA. Ten consecutive pulses were recorded to calculate average PTT values. The error bars represent the measurement standard deviations. d, PWV calculated across five arterial segments using the USoP. e, PWV mapping under normal conditions and cold pressor test. The averaged PWV along each path was calculated from five independent measurements. The error bars indicate the standard deviations of the measured values. The PWV increases from heart-proximal to heart-distal branches. There is a regional increase of PWV in H-BA and BA-RA segments owing to cold-induced vasoconstriction.

FIGS. 10a-10e show the validation metrics of four models on ideal and compromised image datasets. a, The images used for validation including ideal carotid artery images and compromised images (e.g., noise coupled images, artery shifting images, artery missing images). b, The receiver operating characteristic curves validated on 460 ideal images, suggesting the best model VGG13 has an area under the curve value of 100%. c, The precision, recall, and accuracy validated on ideal images. d, The receiver operating characteristic curves validated on 460 images with a mix of ideal and compromised images, suggesting the best model VGG13 has an area under the curve value of 99.4%. e, the precision, recall, and accuracy validated on mixed ideal and compromised images.

FIGS. 11a-11c illustrate the continuous monitoring process during high-intensity interval training (HIIT). a, Photographs showing the participant performing HIIT. Six training sessions, including (i) touch shoulder push-ups, (ii) cycling Russian twist, (iii) push-up rotations, (iv) burpees, (v) side kick through, and (vi) hand-release push-ups. b, The head motions are recorded by the inertia measurement units, which show the rolling, yawing, and pitching rates during the 12 min training and resting. The carotid blood pressure waveforms and heart rate are recorded simultaneously and continuously using the USoP. The systolic pressure increased ˜25 mmHg between training sessions and rest sessions, while the diastolic pressure experienced less fluctuation. c, Zoomed-in view of the head motions, continuous blood pressure waveforms, and heart rate recorded during the training sessions.

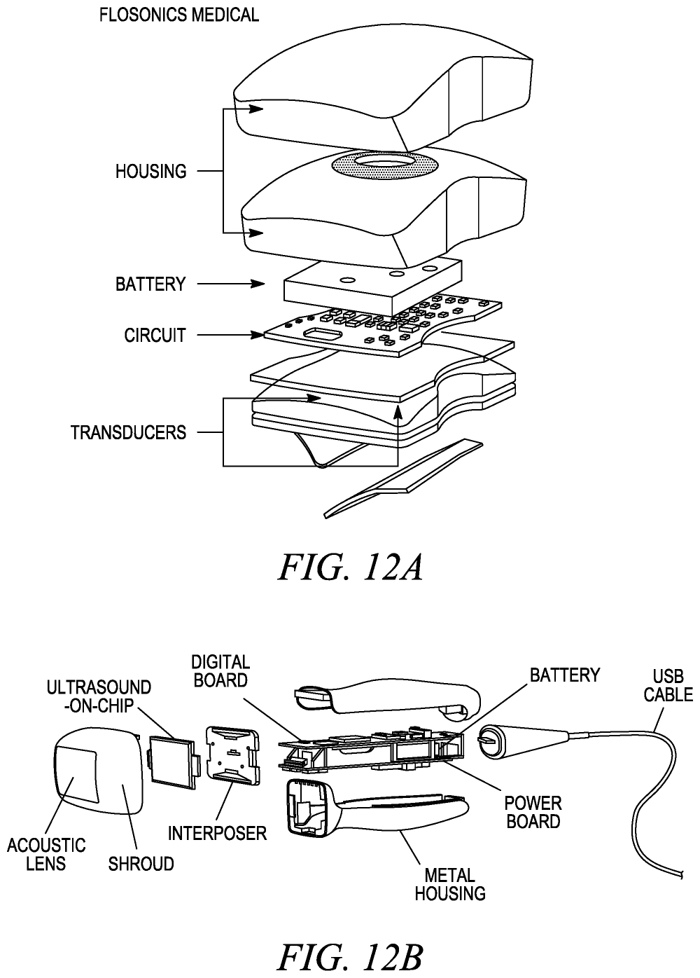

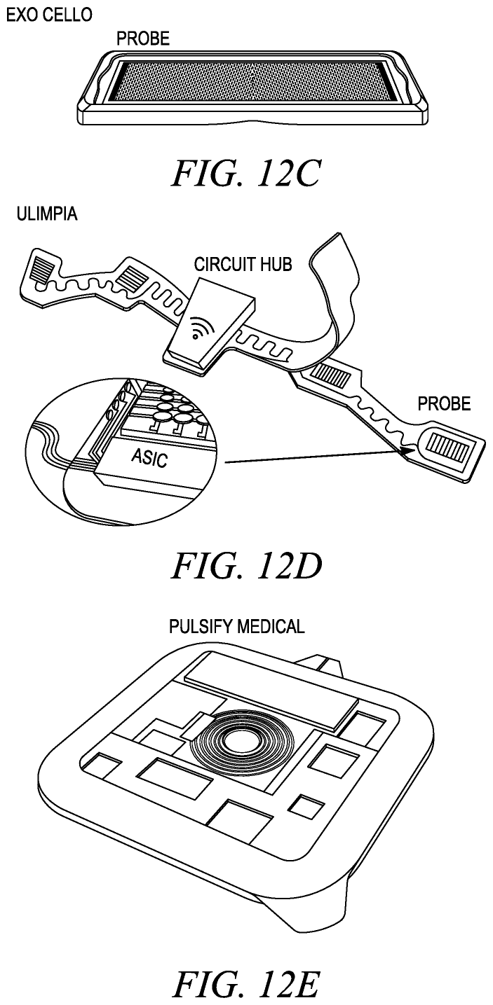

FIGS. 12a-12d show ultrasonic devices for wearable or point-of-care applications. a, The rigid continuous wave Doppler flow sensor developed by Flosonics Medical. b, The rigid hand-held probe developed by Butterfly IQ. c, The rigid piezoelectric micromachined ultrasound transducer (PMUT)-based hand-held probe proposed by Exo Cello. d, The soft ultrasonic imaging device proposed by Ulimipia. e, The soft cardiac monitor proposed by Pulsify Medical.

FIGS. 13a-13b show probe layout designs for reducing noise coupling. a, When the signal electrode faces the skin, the parasitic capacitor Cₛ can directly conduct the in-band noise to the amplifier, resulting in a high noise floor. b, When the ground electrode faces the skin, the capacitor C_{g} will short the noise signals to the ground without interfering with the signal line. As a result, the received radiofrequency signal will have a cleaner baseline.

FIG. 14 demonstrates the improved axial resolution that arises with a backing layer. Without the backing layer, the echo envelope has a full width at half maximum (FWHM) of 1.98 mm. With backing layer, the echo signal has quenched ringing, which results in an improved FWHM of 0.34 mm.

FIGS. 15a-15b show radiofrequency signals collected from the carotid artery with and without gel. The arterial wall echoes acquired with gel (a) and without gel (b) were both strong and distinguishable. The results showed the echo amplitude would decrease by less than 15% when the gel was not applied. Therefore, gel-free measurements experience minimal signal degradation.

FIGS. 16a-16b show results of a durability test of the soft probe. The pulse-echo signals were collected from the neck with the same device. a, The raw radiofrequency signals acquired by a freshly fabricated device and a used device. b, The carotid blood pressure waveform acquired by a new device and a used device.

FIGS. 17a-17e show layout and beam profile designs of three soft probes. a, A cross-sectional view of the stretchable probe design. The transducer and the backing layer are sandwiched by two layers of electrodes (ground (GND) and signal layers). A vertical interconnect access (VIA) is used to lead the ground electrode to the signal layer for connection. b, The two electrodes for the disc probe. The electrodes connect 112 transducers in parallel. c, The two electrodes for the linear array probe. The signal layer consists of 32 channels, and each channel has 8 pixels connected in parallel. d, The two electrodes for the 2D array probe. 32 transducers are grounded by one bottom electrode. The signal layer is distributed into four layers. e, Simulated acoustic transmission fields of the three probe designs, where penetrative, wide, and narrow beam profiles could be achieved by the disc, linear array, and 2D layouts, respectively.

FIGS. 18a-18d show characterization data for the detachable ACF connection. a, The top view of the ACF for detachable connection. b, The cross-sectional schematic diagram showing the hot compression bonding process. The nanoparticles in anisotropic conductive adhesive (ACA) form vertical connections between the copper pad and ACF silver trace after hot compression. Debonding can be achieved by reheating and detaching the ACF and ACA from the copper pad when they are hot. c, Repetitive bonding and debonding were conducted fourteen times to show the reproducibility of the ACF connection. During each round of bonding, eight copper pads were bonded at once, and their impedances were measured. The average impedances were all <10Ω, and minimally increased within 10 times of repetitive bonding and debonding.

FIG. 19a-19c show layout designs of the fPCB circuit. a, Layouts of the fPCB with four layers of interconnects. b, Photos of the fPCB with key components (see Table 1) labeled. The analog front-end is 3 cm×4 cm in size. The wireless data acquisition module is 3 cm×3 cm in size. c, The circuit being bent and twisted to show its flexibility.

FIG. 20 shows schematic connections of the analog front-end and wireless data acquisition module. The analog front-end consists of the pulse generator, receiver, multiplexers, transmit/receive switch (T/R SW), sequencer, and connectors. The wireless data acquisition module consists of a microcontroller (MCU, PIC32) with on-chip analog-to-digital converter (ADC) and a Wi-Fi circuit (ESP32).

FIGS. 21a-21d illustrate the foldability of the fPCB. a, The modular design of the circuitry consisting of the wireless data acquisition (DAQ) and the analog front-end (AFE) modules. The rigid chips with a thickness of more than 0.5 mm are highlighted with colored boxes. b, A zoomed-in view showing the serpentine interconnects between the DAQ and the AFE module. The power supply wires connect the battery voltage (V+) and the ground (GND) between two modules. The AFE outputs radiofrequency (RF) signals, which are received by the DAQ as the input to the analog-to-digital converter (ADC). Meanwhile, the DAQ module outputs trigger signals, which are received by the AFE as the input to initiate pulse-echo sensing. c, The chip layout was designed to reduce the thickness of the fPCB when folded. After folding, the board-to-board spacing is determined by two components (Pin as battery connectors, and inductor L2) with a thickness of 1.75 mm. Note that the overlapped chips (UR1 and U1_1) are of the same 1.75 mm thickness. Thus, the overlap does not add additional thickness to the folded device. d, Side views of the fPCB before and after folding. The folded DAQ and AFE modules have a minimum separation of 1.75 mm. The footprint of the entire fPCB is reduced from 3 cm by 8.3 cm to 3 cm by 4 cm after folding.

FIGS. 22a-22c show designs of the mold for the elastomeric package. a, Three-view drawing and dimensions of the mold for the elastomeric package. b, The 3D printed mold and demolded elastomeric package piece. c, Two packaging strategies for the fPCB. For the first strategy, the fPCB is unfolded and encapsulated by the demolded elastomer piece and a flat substrate piece (left). For the second strategy, the fPCB is folded and wrapped by the demolded elastomer piece for a smaller footprint (right). In both packaging strategies, the packaged USoP would be applied to skin with commercially available medical silicone adhesives.

FIGS. 23a-23e show mechanical simulations of the fPCB and the elastomeric package. a, Top and bottom views of the fPCB. One cross-section of the printed circuit board along the white dashed line is simulated under bending. b, An optical image of the device cross-sectional geometry and the corresponding simulated maximum principal strain distribution in the fPCB. The maximum bending curvature achieved without plastic deformation is 0.14 cm⁻¹, corresponding to a bending angle of 24.1°. The maximum principal strain and von-Mises stress of c, the human skin, d, elastomeric package, and e, fPCB. The simulation results suggest the deformations of the device are elastic under 10% skin stretching. f, An optical image of the packaged device under 10% uniaxial stretching.

FIG. 24 compares the raw signal frequency and circuit sampling rate of representative wearable physiological monitors. According to the Nyquist-Shannon sampling theorem, the circuit sampling rate should be at least two times higher than the raw signal frequency for proper sampling. Thermal, biopotential, accelerometric, photonic, electrochemical, strain, and ultrasonic signals are compared. The USoP device in this work offers more than three orders of magnitude higher circuit sampling rate than the other sensors and thus can capture ultrasonic signals with much higher frequency.

FIGS. 25a-25c illustrate the wireless transmission of the ultrasonic signals via Wi-Fi. a, The testing setup showing data transmission between the USoP and a smartphone. b, The Wi-Fi signal intensity with increasing transmission distance. Within ˜10 m separation, the Wi-Fi intensity can maintain >−60 dBm for reliable transmission. The intensity value was averaged from twenty repetitive measurements, and the error bar represents the standard deviation. c, The transmission speed at 10 m, with a 3.4 Mbps data transmission rate.

FIGS. 26a-26b illustrate the power consumption and battery life of the USoP. a, Current consumption of the circuit components with a 3.7 V input. The total average current consumption is 166 mA (24 mA for the analog front end (AFE) and 142 mA for the wireless data acquisition (DAQ) module). Thus, the power of the USoP is ˜614 mW. b, Lifetimes (upper panel) and the corresponding length (L) width (W), and height (H) (lower panel) of commercial batteries. By increasing the battery capacity and size from 400 mAh, 4.76 cm³ to 2 Ah, 20.29 cm³, the USoP can continuously operate for 2.4 h ˜12.0 h.

FIGS. 27a-27d illustrate multi-mode sensing with wearable ultrasonic probes. a, Amplitude mode (A-mode) for capturing arterial walls. Envelopes of radiofrequency signals indicate the amplitudes and positions of the reflection interfaces. The arterial diameter (d) is then the product of one half of the acoustic time-of-flight (t₂−t₁) and acoustic speed (c). b, Motion mode (M-mode) for capturing the distensions of arterial walls continuously. Exemplary frames of radiofrequency signals (left) with corresponding diastolic and systolic phases in the M-mode image (right). c, Motion mapping of the brachial artery using the 6 MHz 2D probe. Based on the distension amplitudes (left and middle), the spatial orientation of the brachial artery can be mapped (right). d, Brightness mode (B-mode) imaging of an iron wire phantom using a 2 MHz linear array probe. Radiofrequency signals (left) illustrate the reflected wavefront of the iron wire. Reconstructed images (right) show the imaged iron wire at depths of 1 cm, 2 cm, and 3 cm. The axial and lateral full widths at half maximum are labeled on the images showing the imaging resolution of the linear array at different depths.

FIGS. 28a-28g illustrate the lateral and elevational resolution of the soft probes. a, Schematic illustration of the soft probes showing the lateral and elevational direction of resolution characterization. b, The lateral and elevational resolution of a non-imaging array at a certain depth could be defined as the beam width of each transducer. c, The lateral/elevational transmission beam pattern of the 2 MHz single transducer and its beam spreading profiles at 10-30 mm depth. c, The lateral/elevational transmission beam pattern of a single transducer in the 6 MHz 2D array and its beam spreading profiles at 10-30 mm depth. d, The lateral transmission beam pattern of a single transducer in the 4 MHz linear array and its beam spreading profiles at 10-30 mm depth. f, The elevational transmission beam pattern of one sensing channel in the 4 MHz linear array and its beam spreading profiles at 10-30 mm depth. The activated transducers were labeled in the inset photos. g, The −3 dB beam width of the beam patterns showing the lateral and elevational resolutions of three probes.

FIGS. 29a-29e illustrate the transmission beam patterns with elevational deformation. a, Schematics showing two arrays bent at a curvature of 10 mm⁻¹. Both devices have 8 transducers. The small aperture device has a pitch of 0.8 mm, while the large aperture device has a pitch of 1.6 mm. A point source is set at 5 cm away from the array center. b, Corresponding time delay errors were calculated for each transducer. c, Simulated elevational beam patterns of the 4 MHz linear array. The probe was bent elevationally with radii of 5-10 mm and the beam patterns were compared with a flat array. d, Beam intensity profiles at a depth of 5 mm showing the side lobe intensities are <30% of the main lobe at all bending curvatures. e, −3 dB beam width suggesting the bending is not generating significant beam widening when the bending radius is >6 mm.

FIGS. 30a-30b show simulated B-mode images of point sources with azimuthal bending. a, Schematics showing a bent linear array along the azimuthal direction. b, B-mode imaging results of point sources at depths of 1 cm, 1.5 cm, 2 cm, 2.5 cm, and 3 cm by a 4 MHz linear array with different bent radii. The results suggest artifacts (labeled with red arrows) would appear when the array is bent with a radius <6 cm.

FIGS. 31a-31c illustrate tissue interfacial motion detection using the auto-correlation method. a, Two frames of radiofrequency echoes showing the motion of tissue interfaces. b, Segmented radiofrequency echoes containing the reflection from a tissue interface. The envelopes are generated from the echo segments to define the profile of the interfacial reflection. c, Auto-correlation value calculated from the envelopes. A lag of 0.384 μs corresponding to the maximum auto-correlation value is determined as the time delay between the two frames.

FIGS. 32a-32g show probe positions and acoustic views of different bio-interface measurements. The probe positions and viewing angles are labeled in the schematics. B-mode images from a 25-year-old healthy subject were collected using a commercial Butterfly IQ hand-held probe as references. a, Radial artery and b, brachial artery are collected using the default setting for “Vascular Access”. c, Carotid artery and d, femoral artery are collected using the default setting for “Vascular: Carotid”. e, Abdominal aorta is collected using the default setting for “Abdomen”. f, Left ventricle is collected using the default setting for “Cardiac”. g, Diaphragm dome is collected using the default setting for “Abdomen Deep preset”.

FIGS. 33a-33b show fractional shortening measurements using a commercial ultrasonic system. a, A B-mode image showing the parasternal long-axis view of the heart with a cross-sectional view of the left ventricle. b, An M-mode image generated from the center scanning line of the B-mode image in a. The left ventricular internal diameter end systole (LVIDs) and end diastole (LVIDd) can be recorded. The fractional shortening can be calculated as (LVIDs−LVIDd)/LVIDd=30.18% in this case.

FIGS. 34a-34b show calculations of expiratory volumes. a, Diaphragm motion during forced expiration recorded by the USoP. In the exhaling phase, the total excursion (FVC) and the excursion within the first second of exhaling (FEV₁) were recorded. b, Based on the measured FEV₁ and FVC, the respiratory function of a healthy volunteer was evaluated. The volunteer performed the same FEV₁ and FVC measurements after participating in aerobic training ˜5 hours per week for four consecutive months. The four-quadrant plot suggests an increased FVC, indicating an enhanced expiratory function. Unhealthy respiratory performance, such as obstructive, restrictive, and combined conditions, could be diagnosed if the FVC and FEV₁/FVC values are below the lower limit of normal (LLN).

FIGS. 35a-35b illustrate model training and validation with modified datasets. a, Modifications to the original image datasets, including one wall cropped, two walls cropped, and label-shuffled images. b, The VGG13 model validation metrics on these modified datasets. The training/validation was conducted on a modified dataset of 3826 ultrasound images with a 1:1 training/validation split.

FIGS. 36a-36e illustrate the process of classifying carotid artery images by the image processing and logistic model. a, Work flowchart of the logistic model. b, Image processing steps to extract salient edges in the image. c, Pulse detection based on fast Fourier transform. d, Validation metrics comparison between the logistic models and the VGG13 deep learning model. e, Failed edge detection with compromised images, including noise coupling, artery shifting, and artery missing.

FIG. 37 illustrates the statistical validation of the prediction of the best channel for carotid artery sensing against the ground truth. 50 prediction results of the VGG13 model are plotted against the human-determined best channel. The regression function suggests a linear relationship (y=1.004x-0.137) between the prediction and the ground truth. The overlapped data points are plotted as offset crosses.

FIGS. 38a-38c illustrate the process of recording head rotation. a, Two separate inertial measurement units (LSM6DS3) were mounted on the head and chest of the participant to record head rotation. b, The circuit to interface LSM6DS3, which had a memory card to save the recordings for post-processing. c, The recorded yawing rates while the participant was performing torso rotation and head rotation. By calculating the difference between the head unit and the torso unit, the torso motion could be removed and thus, accurate head rotation could be recorded.

FIGS. 39a-39b illustrate carotid artery displacements under head movements. a, Schematic illustration of 3-degree of freedom head rotations, including yawing, rolling, and pitching (left). A typical person can pitch and roll from −40° to +40° and yaw from −80° to +80°. The carotid artery has the largest displacement during head yawing (right). b, The B-mode images collected by a commercial ultrasonic probe showing the displacements under various head rotations. The coordinates labeled in the images are the position of the artery center.

FIGS. 40a-40d illustrate detection of a moving artery using the linear array probe. a, A simulated acoustic beam profile when one sensing channel in the linear array is activated. The beam center has the strongest acoustic intensity. b, Schematic illustration of the carotid artery (CA) cross-section during head movements. The dashed lines represent the beam centers of the sensing channels with the highest acoustic intensity. c, M-mode images recorded by each channel (Ch) while the carotid artery is moving. For each sensing channel, arterial pulses will appear for a period, when the artery is insonated by its acoustic beam. The periods containing arterial pulses are highlighted by white boxes. d, Readings of the sensing channels showing the position of the carotid artery. In this case, the carotid artery can be sensed by channels #13-17, #16-20, and #19-23 at t₁, t₂, and t₃, respectively.

FIGS. 41a-41d illustrate M-mode images collected by one sensing channel with increasing yawing rates. a, Schematic illustration showing the relative positions of the acoustic beam and the moving carotid artery. b, M-mode images collected by one fixed sensing channel at a yawing rate of 20°/s. Three recognizable periods of recording are observed from the M-mode image when the carotid artery passes by. In the beginning, when the carotid artery is outside the sensing channel, no arterial pulses are detected (Period i). Pulses are recorded when the carotid artery moves underneath the sensing channel (Period ii). Finally, the pulse fades when the artery moves out of the sensing channel (Period iii). c, M-mode images with yawing rates increased from 0°/s to 80°/s, showing a decreasing pulse period. When the yawing rate increases to 60°/s, the pulse period is shorter than one heartbeat period, meaning the M-mode image would record less than a full cycle of a pulse d. The averaged pulse period and true positive rate (TPR, true carotid artery image) of M-mode images drop substantially when the yawing rate reaches 60°/s. For each yawing rate, 100 images were used for calculating the averaged pulse period and TPR. The error bars represent the standard deviations of 100 pulse periods extracted from the image.

FIG. 42 shows recorded pulse waveforms under increasing yawing rates from 0°/s to 80°/s. Under slow motions, the carotid pulse waveforms show high continuity. When the yawing rate increases to 70°/s and 80°/s, the waveforms start to show obvious distortions.

FIGS. 43a-43b quantify the domain distance and visualization of the domain distributions. a. The squared log-Euclidean distance, representing the domain distance, decreased with training epoch increase. b. The domain distribution was visualized using the t-distributed stochastic neighbor embedding procedure. Before domain adaptation (Epoch 0), the source domain (subject #1) and the target domain (subject #2) could be easily differentiated. After domain adaptation (Epoch 3600), the two domains merged, showing no significant discrepancies.

FIG. 44 shows a heatmap of the classification accuracy observed after domain adaptation with different numbers of images from the target and source domains. The heatmap shows that a high accuracy (>90%) can be attained by using as few as 32 unlabeled images from the target domain and 67 labeled images from the source domain for domain adaptation training.

FIGS. 45a-45b show representative pressure waveforms recorded during cycling and HIIT. Central blood pressure waveforms recorded during a, cycling and b, HIIT. The waveform morphologies change significantly during exercise sessions. In both exercise scenarios, the difference between the systolic pressure peak and reflection pressure peak increases during exercise, indicating reduced distal reflection and increased vasodilation during exercise.

FIGS. 46a-46b show measurements of the AIx. a, Schematics showing the arterial blood pulse waveform formation and the calculation of the AIx. The forward wave (P₁) and reflected wave (P₂) constitute local peaks in a blood pressure waveform. AIx is calculated as the peak difference divided by the forward peak. There is an additional local minimum point resulting from the closure of the aortic valve (AV). b, Blood pressure waveforms from the carotid artery under resting and post-exercise situations. In a resting situation, the distal end of the arterial tree has a higher impedance, resulting in an early and strong reflection peak P₂. On the contrary, in a post-exercise situation, the distal end has a lower impedance, resulting in a late and mild reflection peak P₂.

FIGS. 47a-47b show measurements of the arterial stiffness index (β) before, during, and after exercise. The β value of each scenario was averaged from twenty independent measurements. The error bar represents the standard deviation. a, The calculated β before, during, and after exercise showing a negligible change of <0.34%. b, During exercise, such a change in β causes a maximum error in blood pressure of 1.58 mmHg.

FIGS. 48a-48b show muscle recruitments and corresponding AIx during cycling and HIIT. a, Different muscle groups are involved during cycling and HIIT. HIIT (i), (iii), and (vi) share the same least muscle activation, during which the pectoralis, deltoids, and triceps are activated. HIIT (v) has the second least muscle activation, during which the deltoids and quadriceps are activated. Cycling has more muscle activation, during which quadriceps, tibialis, and calve are activated. The HIIT (ii) has the second most muscle activation, during which the rectus abdominus, abdominal obliques, and quadriceps are activated. The HIIT (iv) has the most muscle activation, during which all muscle groups mentioned above are activated. b, The calculated AIx during exercise, which increases with increasing the amount of muscles activated during exercise.

FIG. 49 shows an estimation of the stroke volume by the pulse contour method. Two central blood pressure waveforms collected from the carotid artery during rest and exercise. The area under the curve (AUC) of the systolic phase is enlarged, indicating an increased stroke volume during exercise.

FIGS. 50a-50d illustrate acquisition errors in conventional ultrasonography. B-mode and M-mode images are collected from the carotid artery using a commercial Butterfly IQ hand-held probe. a, Clear B-mode (left) and M-mode (right) images collected with stable probe holding, a correct scanning line, and the patient staying still. b, A compromised M-mode image with unstable probe holding. c, Selection of a deviated scanning line in B-mode image (left), resulting in an underrated arterial diameter in the M-mode image (right). d, An M-mode image with motion artifacts due to patient movement.

FIG. 51 is a flowchart illustrating one example of a method for monitoring a physiologic parameter that may be performed by various embodiments of the USoP described herein.

TABLE 1
Major components used in the control electronics. All
of the components are commercially off the shelf.

| Component | | Manufacture product |
|---|---|---|
| designator | Description | number |
| 1, 2 | Multiplexer | MAX14866UTM + T |
| 3 | T/R switch | MD0101K6-G-ND |
| 4 | Operational amplifier | ADA4895-1ARJZ-R7 |
| 5, 6 | Operational amplifier | ADA4897-1ARJZ-RL |
| 7 | Single-pole double-throw | TS5A3159ADBVR |
| | analog switch | |
| 8 | Voltage inverter | MAX829EUK |
| 9 | Zener diode | BZD27B18P-M3-08 |
| 10 | Zener diode | BZX100A |
| 11, 12, 13 | Schottky diode | SB01-15C-TB-E |
| 14, 15 | MOSFET-N | CPH3459-TL-W |
| 16 | Schmitt-trigger inverter | SN74LVC1G14DRLR |
| 17 | Microcontroller | ATMEGA328P-ANR |
| 18 | Voltage regulator | MIC5205-3.3YM5-TR |
| 19 | Voltage regulator | AMS1117 |
| 20 | Microcontroller with ADC | PIC32MZ1024EFH064-I/MR |
| 21 | Voltage regulator | MIC5365-3.3YC5-TR |
| 22 | Wi-Fi module | ESP32-S3-WROOM-1 |

TABLE 2
The typical depths and motion magnitudes of different tissue
interfaces. The interfaces in this study include the arterial
walls, ventricular wall, and diaphragm dome.

| Tissue interface | Depth | | Motion scale |
|---|---|---|---|
| Radial artery wall | 1.00-4.00 | mm | 0.01-0.06 mm |
| Brachial artery wall | 3.0-8.1 | mm | 0.04-0.17 mm |
| Common carotid artery wall | 4.4-30.4 | mm | 0.26-0.90 mm |
| Common femoral artery wall | 10-140 | mm | 0.15 mm-1.00 mm |
| Abdominal aorta wall | 40-100 | mm | 0.57-2.00 mm |
| Ventricular wall | 69.9-92.7 | mm | 12.2-16.2 mm |
| Diaphragm | 100-181.7 | mm | 8.0-42.0 mm (normal |
| | | | breath) |
| | | | 52.7-92.1 mm (forced |
| | | | breath) |

TABLE 3
Summary of typical expiratory volumes and their measurements.
Clinical measurements of FVC, FEV₁, and the derived parameter
FEV₁/FVC are used for diagnosing different respiratory issues.

| Full name | Clinical measurements |
|---|---|
| Forced vital | Total volume achieved by the quickest possible |
| capacity (FVC) | exhalation after a maximal inhalation |
| Forced expiratory | Volume achieved in the first second by the quickest |
| volume in one | possible exhalation after a maximal inhalation |
| second (FEV₁) | |
| FEV₁/FVC | Forced expiratory volume measured in the first |
| | second as a percentage of forced vital capacity |

TABLE 4
Demographic characteristics of the participants in this study. They
vary in gender, race, age, height, weight, and body-mass index,
which generate diversity in the collected ultrasonic images.

| Gender | n (percentage) |
|---|---|
| Male | 6 (60%) |
| Female | 4 (40%) |
| Race | n (percentage) |
| Asian | 5 (50%) |
| Hispanic or Latino | 3 (30%) |
| White | 2 (20%) |
| At time of study | Mean ± Standard deviation |
| Age (years) | 27.78 ± 4.50 |
| Height (cm) | 171.04 ± 10.88 |
| Weight (kg) | 64.26 ± 10.27 |
| Body-mass index (kg/m²) | 21.78 ± 2.52 |

Certain aspects of subject matter described herein, such as the USoP and the back-end computing environment, are presented in the foregoing description and illustrated in the accompanying drawing using electronic hardware, computer software, or any combination thereof. Whether such elements are implemented as hardware or software depends upon the particular application and design constraints imposed on the overall system. By way of example, such elements, or any portion of such elements, or any combination of such elements may be implemented with one or more processors or controllers. Examples of processors or controllers include microprocessors, microcontrollers, digital signal processors (DSPs), field programmable gate arrays (FPGAs), programmable logic devices (PLDs), state machines, gated logic, discrete hardware circuits, and any other suitable hardware configured to perform the various functionalities described throughout this disclosure. Examples of processors or controllers may also include general-purpose computers or computing platforms selectively activated or reconfigured by computer-executable instructions such as computer programs to provide the necessary functionality.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalent of the appended claims.

1. A system for monitoring a physiologic parameter, comprising:
a. a conformal ultrasonic transducer array located on a flexible substrate;
b. an analog front end circuit located on the flexible substrate and further coupled to the conformal ultrasonic transducer array, the analog front end circuit configured to at least cause the conformal ultrasonic transducer array to generate ultrasonic acoustic waves and receive reflected ultrasonic acoustic waves;
c. a digital circuit located on the flexible substrate and further coupled to the analog front end circuit, the digital circuit configured to at least: i. control the analog front end circuit at least in its generation of ultrasonic acoustic waves using a plurality of sensing channels; ii. transmit data concerning the received reflected ultrasonic acoustic waves to a back-end computing environment that dynamically selects a monitoring channel in real-time from among the plurality of sensing channels; and iii. receive in real-time at least an identifier of the selected monitoring channel from the back-end computing environment and cause the analog front-end circuit to generate ultrasonic acoustic waves using the selected monitoring channel to perform the monitoring of the physiological parameter.

2. The system of claim 1 wherein the selected monitoring channel received by the digital circuit is dynamically selected in real-time by the back-end computing environment at least in part using artificial intelligence techniques.

3. The system of claim 2 wherein the artificial intelligence techniques identify sensing channels that cause reflected ultrasonic acoustic waves to be reflected from specified tissue that is to be monitored.

4. The system of claim 2 wherein the artificial intelligence techniques identify sensing channels that cause ultrasonic acoustic waves to be transmitted through specified tissue that is to be monitored.

5. The system of claim 2 wherein the artificial intelligence techniques employ models that are generalizable to allow physiological monitoring to be performed on different subjects.

6. The system of claim 1 wherein the selected monitoring channel received by the digital circuit is dynamically selected in real-time by the back-end computing environment to accommodate motion of tissue relative to the conformal ultrasonic transducer array.

7. The system of claim 1 wherein the physiological parameter being monitored is selected from the group including blood pressure, heart rate, pulse wave velocity, stroke volume, cardiac output, augmentation index, and expiratory volume.

8. The system of claim 1 wherein the digital circuit includes a wireless communication circuit for communicating with the back-end computing environment.

9. The system of claim 8 wherein the wireless communication circuit is a Wi-Fi communication circuit.

10. The system of claim 1 wherein the digital circuit is further configured to transmit an indication of the reflected ultrasonic acoustic waves arising from use of the selected monitoring channel to an external computing environment for display thereon.

11. The system of claim 1 wherein the digital circuit is further configured to present an indication of the reflected ultrasonic acoustic waves arising from use of the selected monitoring channel.

12. The system of claim 11 wherein the back-end computing environment is the same or different from the external computing environment.

13. The system of claim 2, further comprising the backend computing environment, wherein the backend computing environment is configured to measure a shift, the shift in the time domain, in a detected peak of the received reflected acoustic wave, the shift due to movement of an organ or tissue, and wherein the displayed indication of the monitored physiologic parameter is based on the measured shift.

14. The system of claim 1, wherein the analog front end is further configured to steer or direct the generated ultrasonic acoustic waves toward an organ, tissue, or location of interest, the steering or directing by beamforming.

15. The system of claim 14, wherein the steering includes dynamically adjusting a time-delay profile of individual transducer activation in the transducer array.

16. The system of claim 1, wherein the transducer array is selected from the group including a piezoelectric array, a piezoelectric micromachined ultrasonic transducer (PMUT) array or a capacitive micromachined ultrasonic transducer (CMUT) array.

17. The system of claim 1 wherein the analog front end circuit includes a multiplexer for selecting from among all sensing channels that are used to generate the ultrasonic acoustic wave and perform monitoring.

18. The system of claim 2 wherein the artificial intelligence techniques are machine learning techniques.

19. A system for monitoring a physiologic parameter, comprising:
a. a conformal ultrasonic transducer array located on a flexible substrate;
b. an analog front end circuit located on the flexible substrate and further coupled to the conformal ultrasonic transducer array, the analog front end circuit configured to at least cause the conformal ultrasonic transducer array to generate ultrasonic acoustic waves and receive reflected and/or transmitted ultrasonic acoustic waves;
c. a digital circuit located on the flexible substrate and further coupled to the analog front end circuit, the digital circuit configured to at least: i. control the analog front end circuit at least in its generation of ultrasonic acoustic waves using a plurality of sensing channels; ii. dynamically select a monitoring channel in real-time from among the plurality of sensing channels; and iii. cause the analog front-end circuit to use the selected monitoring channel to perform the monitoring of the physiological parameter.

20. A method for monitoring a physiologic parameter, comprising:
a. determining a location of interest, the location associated with the physiologic parameter to be monitored;
b. transmitting ultrasonic acoustic waves toward the location of interest and receiving reflected ultrasonic acoustic waves from the location of interest using a plurality of sensing channels;
c. dynamically selecting a monitoring channel in real-time from among the plurality of sensing channels;
d. monitoring the physiological parameter in real-time by transmitting ultrasound acoustic waves toward the location of interest and receiving reflected ultrasonic acoustic waves using the selected monitoring channel;
e. outputting data reflective of the monitored physiological parameter; and
f. wherein at least steps (b) and (d) are performed by components within the conformable integrated wearable device.

21. The method of claim 20 wherein step (c) is also performed by components within the integrated conformable wearable device.

22. The method of claim 20 wherein step (c) is performed by a back-end computing environment located external to the integrated conformable wearable device and further comprising:
transmitting data concerning the received reflected/transmitted ultrasonic waves from the conformable integrated wearable device to the back-end computing device; and
receiving from the back-end computing device at least an identifier of the selected monitoring channel.

23. The method of claim 20 wherein the selected monitoring channel is dynamically selected in real-time at least in part using artificial intelligence techniques.

24. The method of claim 23 wherein the artificial intelligence techniques identify sensing channels that cause reflected ultrasonic acoustic waves to be reflected from specified tissue that is to be monitored.

25. The method of claim 23 wherein the artificial intelligence techniques identify sensing channels that cause ultrasonic acoustic waves to be transmitted to specified tissue that is to be monitored.

26. The method of claim 23 wherein the artificial intelligence techniques employ models that are generalizable to allow physiological monitoring to be performed on different subjects.

27. The method of claim 20 wherein the selected monitoring channel is dynamically selected in real-time to accommodate motion of tissue relative to the conformal ultrasonic transducer array.

28. The method of claim 20 wherein the physiological parameter being monitored is selected from the group including blood pressure, heart rate, stroke volume, cardiac output, augmentation index, and expiratory volume.

29. The method of claim 22 wherein the integrated conformable wearable device includes a wireless communication circuit for communicating with the back-end computing environment.

30. The method of claim 29 wherein the wireless communication circuit is a Wi-Fi communication circuit.

31. The method of claim 20 wherein the displaying includes transmitting an indication of the reflected ultrasonic acoustic waves arising from use of the selected monitoring channel to an external computing environment for display thereon.

32. The method of claim 21, further comprising measuring a shift, the shift in the time domain, in a detected peak of the received reflected acoustic wave, the shift due to movement of an organ or tissue, and wherein the displaying of data reflective of the monitored physiologic parameter is based on the measured shift.

33. The method of claim 20, further comprising steering or directing the transmitted ultrasonic acoustic waves toward an organ, tissue, or location of interest, the steering or directing by beamforming.

34. The method of claim 23 wherein the artificial intelligence techniques are machine learning techniques.

35. The method of claim 20 wherein the outputting of data reflective of the monitored physiological parameter includes displaying data reflective of the monitored physiological parameter.

36. A method for monitoring a physiologic parameter, comprising:
a. determining a location of interest, the location associated with the physiologic parameter to be monitored;
b. transmitting ultrasonic acoustic waves toward the location of interest and receiving resulting ultrasonic acoustic waves transmitted through the location of interest using a plurality of sensing channels;
c. dynamically selecting a monitoring channel in real-time from among the plurality of sensing channels;
d. monitoring the physiological parameter in real-time by transmitting ultrasound acoustic waves toward the location of interest and receiving resulting ultrasonic acoustic waves transmitted through the location of interest using the selected monitoring channel;
e. outputting data reflective of the monitored physiological parameter; and
f. wherein at least steps (b) and (d) are performed by components within the conformable integrated wearable device.

37. The system of claim 1 further comprising a battery located on the flexible substrate for powering the analog front end circuit and the digital circuit.

38. The system of claim 37 wherein the battery is a lithium-polymer battery configured to power the analog front end circuit and the digital circuit up to 12 hours.

39. The system of claim 19 further comprising a. battery located on the flexible substrate for powering the analog front end circuit and the digital circuit.

40. The system of claim 39 wherein the battery is a lithium-polymer battery configured to power the analog front end circuit and the digital circuit up to 12 hours.

41. The system of claim 16 wherein the transducer array has a center frequency between 2 MHz and 6 MHz.
